(19) United States
(12) Patent Application Publication
(10) Pub. No.: US 2024/0342279 A1
(43) Pub. Date: Oct. 17, 2024

(54) Glycan-Interacting Compounds and Methods of Use

(71) Applicant: Seagen Inc., Bothell, WA (US)

(72) Inventors: Ana Paula Galvao da Silva, San Diego, CA (US); Julie DeSander, Arlington, MA (US); Jeffrey Behrens, Newton, MA (US); Darius Ghaderi, Laupheim (DE); Mai Zhang, Carlsbad, CA (US); Kristan Meetze, Lexington, MA (US)

(73) Assignee: Seagen Inc., Bothell, WA (US)

(21) Appl. No.: 18/507,995

(22) Filed: Nov. 13, 2023

Related U.S. Application Data
(63) Continuation of application No. 18/053,155, filed on Nov. 7, 2022, now abandoned.
Application No. 18/053,155 is a continuation of application No. 16/742,434, filed on Jan. 14, 2020, now abandoned.
Application No. 16/742,434 is a continuation of application No. 15/518,214, filed on Apr. 10, 2017, now abandoned.
(60) Provisional application No. 62/173,555, filed on Jun. 10, 2015.
(60) Provisional application No. 62/102,545, filed on Jan. 12, 2015.
(60) Provisional application No. 62/062,474, filed on Oct. 10, 2014.

(51) Int. Cl. A61K 39/395; A61K 31/401; A61K 39/00; A61P 35/00; C07K 16/30; C07K 16/44

(52) U.S. Cl. CPC A61K 39/39558; C07K 16/3015; C07K 16/3046; C07K 16/44; A61K 31/401; A61K 2039/505; A61P 35/00; C07K 2317/622; C07K 2317/73; C07K 2317/732; C07K 2317/77

(57) ABSTRACT

The present invention provides glycan-interacting antibodies and methods for producing glycan-interacting antibodies useful in the treatment and prevention of human disease, including cancer. Such glycan-interacting antibodies include monoclonal antibodies, derivatives, and fragments thereof as well as compositions and kits comprising them. In some embodiments, the present invention provides an antibody having a heavy chain with an amino acid sequence comprising at least 95% sequence identity to SEQ ID NO: 63 and having a light chain with an amino acid sequence comprising at least 95% sequence identity to SEQ ID NO: 64.

### CROSS REFERENCE TO RELATED APPLICATIONS

This application claims priority to U.S. Provisional Patent Application No. 62/062,474 filed Oct. 10, 2014 entitled Glycan-Interacting Compounds and Methods of Use, U.S. Provisional Patent Application No. 62/102,545 filed Jan. 12, 2015 entitled Glycan-Interacting Compounds and Methods of Use, and U.S. Provisional Patent Application No. 62/173,555 filed Jun. 10, 2015 entitled Glycan-Interacting Compounds and Methods of Use, the contents of each of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 9, 2015, is named 2033_1011PCT_SEQ_LIST.txt and is 52,672 bytes in size.

### FIELD OF THE INVENTION

This invention relates to methods for the development of compounds and compositions, including, but not limited to antibodies for the detection and/or removal of glycosylated matter from an organism.

### BACKGROUND OF THE INVENTION

Aberrant glycosylation accompanies some of the other mutations commonly observed in carcinomas. It has been estimated that about 80% of all carcinomas express a truncated glycan, the Tn Antigen. With few exceptions, Tn and the sialylated form Sialyl Tn (STn), are not expressed in normal, healthy tissues. Furthermore, the non-human immunogenic sialic acid, N-glycolylneuraminic acid (Neu5Gc), seems to be differentially expressed on carcinomas such as breast cancer in the form of Neu5Gc-STn (GcSTn).

Multiple aberrant glycosylation forms have been described in human cancers, identifying specific glycans as a class of cell surface molecules suitable for specific tumor targeting (Cheever, M. A. et al., Clin Cancer Res. 2009 Sep. 1; 15(17):5323-37). For example, various human cancer types (such as bladder, breast, cervical, colon, lung, and ovarian cancer among others) show high expression of STn antigen, which is rare in normal human tissues (Karlen, P. et al., Gastroenterology. 1998 December; 11 5(6):1395-404; Ohno, S. et al, Anticancer Res. 2006 November-December; 26(6A):4047-53). In addition, the presence of STn on tumor-associated mucins relates to cancer with poor prognosis and is therewith considered an attractive epitope for cancer detection and targeted therapy (Cao, Y. et al., Virchows Arch. 1997 September; 431(3):159-66; Julien, S. et al., Br J Cancer. 2009 Jun. 2; 100(11):1746-54; Itzkowitz, S. H. et al., Cancer. 1990 Nov. 1; 66(9):1960-6; Motoo, Y. et al., Oncology. 1991; 48(4):321-6; Kobayashi, H. et al., J Clin Oncol. 1992 January; 10(1):95-101). Tn and STn formation is associated with somatic mutations in the gene Cosmc that encodes a molecular chaperon required for the formation of the activate T-synthase (Ju, T. et al., Nature. 2005 Oct. 27; 437(7063):1252; Ju, T. et al., Cancer Res. 2008 Mar. 15; 68(6):1636-46). It can also result from increased expression of the sialyl transferase, ST6GalNAc-1 (Ikehara, Y. et al., Glycobiology. 1999 November; 9(11):1213-24; Brockhausen, I. et al., Biol Chem. 2001 February; 382(2):219-32). De-novo expression of STn can modulate carcinoma cells, change the malignant phenotype, and lead to more aggressive cell behaviors (Pinho, S. et al., Cancer Lett. 2007 May 8; 249(2):157-70). Although STn is highly expressed in malignant tissues, low levels are also found on healthy human cells (Jass, J. R. et al., J Pathol. 1995 June; 176(2):143-9; Kirkeby, S. et al., Arch Oral Biol. 2010 November; 55(11):830-41). STn alone has attracted attention as a target for cancer detection and therapy (Cheever, M. A. et al., Clin Cancer Res. 2009 Sep. 1; 15(17):5323-37).

In addition to the presence of STn, other glycosylation changes have been described in cancer. One of them involves Neu5Gc. N-acetylneuraminic acid (Neu5Ac) and Neu5Gc are the two major sialic acids on mammalian cell surfaces. Neu5Ac and Neu5Gc differ only in that Neu5Gc comprises an additional oxygen atom associated with chemical group attached to carbon 5. Due to the loss of a functional gene, humans can only synthesize sialic acid in the form of Neu5Ac, but not Neu5Gc. However Neu5Gc can be metabolically incorporated into humans from animal-derived dietary sources such as red meats (Tangvoranuntakul, P. et al., Proc Natl Acad Sci USA. 2003 Oct. 14; 100(21):12045-50; Nguyen, D. H. et al., J Immunol. 2005 Jul. 1; 175(1):228-36; U.S. Pat. Nos. 7,682,794, 8,084,219, US2012/0142903, WO2010030666 and WO2010030666, herein incorporated by reference in their entirety). Neu5Gc is significantly abundant among human tumors (Higashi, H. et al., Cancer Res. 1985 August; 45(8):3796-802; Miyoshi I. et al., Mol Immunol. 1986. 23: 631-638; Hirabayashi, Y. et al., Jpn J Cancer Res. 1987. 78: 614-620; Kawachi. S, et al., Int Arch Allergy Appl Immunol. 1988. 85: 381-383; Devine, P. L. et al., Cancer Res. 1991. 51: 5826-5836; Malykh, Y. N. et al, Biochimie. 2001. 83: 623-634 and Inoue, S. et al., 2010. Glycobiology. 20(6): 752-762) and remarkably low in normal human tissues, which had been overlooked for several decades (Diaz, S. L. et al., PLoS One. 2009. 4: e4241; Tangvoranuntakul, P. et al., Proc Natl Acad Sci USA. 2003. 100: 12045-12050; Varki, A. et al., Glycoconj J. 2009. 26: 231-245). The increased metabolic accumulation of diet-derived Neu5Gc in cancer tissue compared to healthy human tissues is likely explained by at least three factors: rapid growth with underproduction of competing endogenous Neu5Ac, enhanced macropinocytosis induced by growth factors (Dharmawardhane, S. et al., Mol Biol Cell. 2000 October; 11(10):3341-52; Simonsen, A. et al., Curr Opin Cell Biol. 2001 August; 13(4):485-92; Johannes, L. et al., Traffic. 2002 July; 3(7):443-51; Amyere, M. et al., Int J Med Microbiol. 2002 February; 291(6-7):487-94), and the upregulation of gene expression of the lysosomal sialic acid transporter gene sialin by hypoxia (Yin, J. et al., Cancer Res. 2006 Mar. 15; 66(6):2937-45). In addition, all humans tested to date comprise a polyclonal antibody reservoir against non-human Neu5Gc, which makes it the first example of a xeno-autoantigen (Padler-Karavani, V. et al., Glycobiology. 2008 October; 18(10):818-30; Varki, N. M. et al., Annu Rev Pathol. 2011; 6:365-93). The accumulation of dietary Neu5Gc in malignant tumors in the face of an anti-Neu5Gc response was shown to facilitate tumor progression by inducing a low-grade chronic inflammation (Hedlund, M. et al., Proc Natl Acad Sci USA. 2008 Dec. 2; 105(48):18936-41). Thus, Neu5Gc containing glycan epitopes on human tumors represent a valuable possibility for drug targeting. A recent study suggests the existence of antibodies against Neu5Gc-containing STn (GcSTn), but not Neu5Ac-STn (AcSTn), in cancer patients and explores their potential as a specific biomarker for cancer detection (Padler-Karavani, V. et al., Cancer Res. 2011 May 1; 71(9):3352-63).

There remains a need in the art for antibodies capable of binding glycans, including glycans associated with disease and diseased cells and tissues. One such antibody is antibody 3F1 (also referred to as HB-STn), which has been described and used to identify cells and structures with STn (see Marcos, N. T., 2004. Cancer Res. 64(19): 7050-7 and Julien, S. et al., 2001. Glycoconjugate Journal. 18: 883-93). Provided herein are variants of 3F1 that have been reformatted and/or optimized for therapeutic applications.

### SUMMARY OF THE INVENTION

In some embodiments, the present invention provides an antibody having a heavy chain with an amino acid sequence comprising at least 95% sequence identity to SEQ ID NO: 63 and having a light chain with an amino acid sequence comprising at least 95% sequence identity to SEQ ID NO: 64. Such antibodies may specifically target a glycan comprising N-acetylneuraminic sialyl Tn antigen (AcSTn) and/or N-glycolylneuraminic sialyl Tn antigen (GcSTn). Some such antibodies may be capable of binding to a cluster of one or more glycans. Further antibodies may be capable of binding a tumor-associated carbohydrate antigen (TACA), in some cases on the surface of one or more cells.

In some embodiments, the invention provides antibody-drug conjugates (ADCs). Such ADCs may comprise antibodies comprising at least one variable domain amino acid sequence with at least 95% sequence identity to the variable domain of SEQ ID NO: 59 or 60. Further ADCs may comprise one or more therapeutic compounds or one or more cytotoxic agents. These may be conjugated directly or via a linker. Cytotoxic agents may include cytoskeletal inhibitors or DNA damaging agents.

In some embodiments, the invention provides bispecific antibodies comprising a first Fab region and a second Fab region wherein the first Fab region comprises the heavy chain variable domain (VH) of SEQ ID NOs: 59 and the light chain variable domain (VL) of SEQ ID NO: 60 and wherein the second Fab region comprises a VH that is not SEQ ID NO: 59 and a VL that is not SEQ ID NO: 60. In some cases, the second Fab region may comprise a VH selected from any of SEQ ID NOs: 1, 4, 6, 8, 10, 12, and 13; and a VL selected from any of SEQ ID NOs: 2, 3, 5, 7, 9, 11, and 14. In some cases, the second Fab region binds a glycan. In some cases, the second Fab region binds to a non-glycan (e.g. a protein).

In some cases, antibodies of the invention comprise intrabodies or chimeric antigen receptors.

In some embodiments, the present invention provides methods of killing one or more tumor cells comprising the use of antibodies of the present invention. Such antibodies may induce antibody-dependent cell-mediated cytotoxicity (ADCC) and/or antibody-dependent cell phagocytosis (ADCP). In some cases, methods of killing may include the use of antibodies conjugated with one or more cytotoxic agent such as monomethyl auristatin E (MMAE) or monomethyl auristatin F (MMAF).

In some embodiments, the present invention provides methods of treating subjects comprising the use of antibodies of the invention. Such subject may include subjects with cancer. In some cases, the cancer is an epithelial cancer selected from breast, colon, lung, bladder, cervical, ovarian, stomach, prostate, and liver cancer. According to some methods, antibodies of the invention may be used to reduce tumor volume in a subject, wherein tumor volume is reduced by at least 1%, at least 5%, at least 25%, at least 50% or at least 75%.

The invention further provides compositions comprising antibodies described herein. These may be comprised in kits further comprising instructions for use.

In some embodiments, the invention provides methods of reducing tumor volume in a subject using one or more antibodies of the invention.

Also provided herein are methods of increasing anti-tumor cell immune activity by providing one or more antibodies of the invention and contacting at least one immune-resistant tumor cell and/or tumor cell microenvironment with such antibodies. Such anti-tumor cell immune activity may include innate immune activity (e.g. natural killer (NK) cell anti-tumor cell activity) or adaptive immune activity (e.g. B cell anti-tumor cell activity and/or dendritic cell (DC) anti-tumor cell activity). Where adaptive immune activity is increased, such increased immune activity may comprise DC anti-tumor cell activity comprising increased DC expression of CD80, CD86, IL-12 and/or TNF-α.

In some embodiments, the present invention provides a method of treating a subject comprising at least one immune-resistant tumor cell by providing one or more antibodies.

The invention further provides constructs encoding one or more amino acid sequences with at least 95% sequence identity to one or more of SEQ ID NOs: 59-64. In some cases, such constructs may comprise the nucleotide sequence of SEQ ID NO: 65 and/or 66 or a variant thereof with at least 95% sequence identity. Constructs may encode antibodies, intrabodies or chimeric antigen receptors of the invention. In some cases, constructs of the invention may be part of a cell or virus.

### BRIEF DESCRIPTION OF THE FIGURES

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIGS. 1A-1D are diagrams depicting α2,6-sialylated N-acetylgalactosamine (STn) and indicating putative epitopes involved in anti-STn antibody binding. The largest ellipse in each diagram indicates the specific region of STn targeted by each of 4 antibody groups. These groups include Group 1 antibodies (binding to the large elliptical region indicated in FIG. 1A), Group 2 antibodies (binding to the large elliptical region indicated in FIG. 1B), Group 3 antibodies (binding to the large elliptical region indicated in FIG. 1C) and Group 4 antibodies (binding to the large elliptical region indicated in FIG. 1D).

FIG. 2A is a histogram illustrating internalization of antibodies of the invention.

FIG. 2B presents microscopy images illustrating internalization of antibodies of the invention.

FIG. 3A is a line graph illustrating antibody-drug conjugate experimental results.

FIG. 3B is a line graph illustrating antibody-drug conjugate experimental results. FIG. 3C is a line graph illustrating antibody-drug conjugate experimental results.

FIG. 4A is a microscope image depicting immunostained tissue sections, FIG. 4B is a microscope image depicting immunostained tissue sections, and FIG. 4C is a microscope image depicting immunostained tissue sections.

### DETAILED DESCRIPTION

### Introduction

In some embodiments, the present invention provides antibodies that are variants of antibody 3F1. 3F1 (also known as HB-STn) binds sialyl-Tn antigen (STn), and has been used to identify cells and structures comprising STn (see Marcos, N. T., 2004. Cancer Res. 64(19): 7050-7 and Julien, S. et al., 2001. Glycoconjugate Journal. 18: 883-93, the contents of each of which are herein incorporated by reference in their entirety).

In nature, STns may be sialylated with N-acetylneuraminic acid (Neu5Ac) or N-glycolylneuraminic acid (Neu5Gc). Glycan-interacting antibodies (such as 3F1) according to the present invention may be directed to glycans comprising any STns (pan-STn antibodies), glycans comprising STns comprising Neu5Ac specifically (AcSTn) or glycans comprising STns comprising Neu5Gc specifically (GcSTn). In some embodiments, glycan-interacting antibodies of the present invention target cancer-related glycan antigens, including those comprising α2,6-sialylated N-acetylgalactosamine (STn).

In some embodiments, the present invention provides methods of using anti-STn antibodies for diagnostic or therapeutic purposes. In some cases, antibodies of the invention are conjugated with cellular effectors such as drugs (e.g., cytotoxic drugs). Antibody drug conjugates (ADCs), when conjugated with a cytotoxic agent, may be used to kill or otherwise reduce proliferation of cells expressing STn. In some cases, antibodies of the invention may be used as biotherapeutics.

Further provided are methods of optimizing, humanizing and using glycan-interacting antibodies disclosed herein. Additionally, kits, assays, and reagents comprising antibodies and/or methods of the present invention are presented. Other embodiments provide methods for generating such glycan-interacting antibodies.

### Definitions

Adjacent: As used herein, the term “adjacent” refers to something that is adjoining, neighboring or next to a given entity. In some embodiments, “adjacent residues” are sugar residues within a glycan chain that are linked to one another. In some embodiments, “adjacent glycans” are glycan chains that next to each other either in direct contact or within close proximity and without another glycan in between the two.

Administered in combination: As used herein, the term “administered in combination” or “combined administration” means that a subject is simultaneously exposed to two or more agents administered at the same time or within an interval of time such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more glycan-interacting antibodies, as described herein, are spaced sufficiently closely together such that a combinatorial (e.g., a synergistic) effect is achieved.

Amino acid: As used herein, the terms “amino acid” and “amino acids” refer to all naturally occurring L-alpha-amino acids as well as non-naturally occurring amino acids. Amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: As used herein, the term “antibody” is used in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications such as with sugar moieties.

Antibody fragment: As used herein, the term “antibody fragment” refers to a portion of an intact antibody, preferably comprising an antigen binding region thereof. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. glycan-interacting antibodies may comprise one or more of these fragments. For the purposes herein, an antibody may comprise a heavy and light variable domain as well as an Fc region.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biomolecule: As used herein, the term “biomolecule” is any natural molecule which is amino acid-based, nucleic acid-based, carbohydrate-based or lipid-based, and the like.

Bispecific antibody: As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

Branch: As used herein, the term “branch” refers to an entity, moiety or appendage that is linked or extends out from a main entity or source. In some embodiments, a “branch chain” or “branching chain” comprises one or more residues (including, but not Hinted to sugar residues) that extend from a parent chain. As used herein, a “parent chain” is used to refer to a chain of residues (including, but not limited to sugar residues) from which a branching chain is linked. In the case of a glycan with multiple branches, the parent chain may also refer to the source chain from which all such branches are directly or indirectly attached. In the case of a polysaccharide comprising a chain of hexose residues, parent chain linkages typically occur between carbons 1 and 4 of adjacent residues while branching chains are attached to a parent chain through a linkage between carbon 1 of the branching residue and carbon 3 of the parent residue from which the branch extends. As used herein, the term “branching residue” refers to the residue attached to the parent chain in a branching chain.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Cyclic or Cyclized: As used herein, the term “cyclic” refers to the presence of a continuous loop. Cyclic molecules need not be circular, only joined to form an unbroken chain of subunits.

Cytidine monophosphate-N-acetylneuraminic acid hydroxylase: As used herein, the term “cytidine monophosphate-N-acetylneuraminic acid hydroxylase” or “CMAH” refers to an enzyme, absent in humans, but present in most other mammals (including, but not limited to mice, pigs and chimpanzees) that catalyzes the formation of N-glycolylneuraminic acid from N-acetylneuraminic acid. The absence of the enzyme in humans is due to a frameshift mutation resulting in the premature termination of the CMAH transcript and the production of a non-functional protein.

Cytotoxic: As used herein, the term “cytotoxic” is used to refer to an agent that kills or causes injurious, toxic, or deadly effects on a cell (e.g., a mammalian cell (e.g., a human cell)), bacterium, virus, fungus, protozoan, parasite, prion, or a combination thereof.

Delivery: As used herein, “delivery” refers to the act or manner of transporting a compound, substance, entity, moiety, cargo or payload to an intended destination.

Delivery Agent: As used herein, “delivery agent” refers to any substance which facilitates, at least in part, the in vivo delivery of a compound, substance, entity, moiety, cargo or payload.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Display library: As used herein, the term “display library” refers to a tool used in scientific discovery to identify biomolecular interactions. Different variations of display libraries exist that include the utilization of bacteriophages, yeast and ribosomes. In each case, proteins within a given library (also referred to herein as “library members”) are linked (physically or through association with a host) to the nucleic acid which encodes the protein. When a target molecule is incubated with the members of a display library, any library members that bind to the target may be isolated and the sequences encoding the bound protein may be determined through analysis of the linked nucleic acid. In some embodiments, display libraries are “phage display libraries” wherein the display library is made up of bacteriophage viral particles (also referred to herein as “phage particles”) wherein nucleic acids have been incorporated into the phage genome resulting in the production of viral coat proteins that are fused to proteins encoded by the nucleic acids that have been introduced. Such fused proteins are “displayed” on the outer surface of the assembled phage particles where they may interact with a given target.

Distal: As used herein, the term “distal” means situated away from the center or away from a point or region of interest.

Engineered: As used herein, embodiments of the invention are “engineered” when they are designed to have a feature or property, whether structural or chemical, that varies from a starting point, wild type or native molecule. Thus, engineered agents or entities are those whose design and/or production include an act of the hand of man.

Epitope: As used herein, an “epitope” refers to a surface or region on a molecule that is capable of interacting with components of the immune system, including, but not limited to antibodies. In some embodiments, an epitope may comprise a target site. Epitopes may comprise a region on an antigen or between two or more antigens that is specifically recognized and bound by a corresponding antibody. Some epitopes may comprise one or more sugar residues along one or more glycan. Such epitopes may comprise 1, 2, 3, 4, 5, 6, 7, 8, 9 or at least 10 sugar residues. Epitopes may also comprise one or more regions of interaction between entities. In some embodiments, epitopes may comprise a junction between two sugar residues, between a branching chain and a parent chain or between a glycan and a protein.

Ether bond: As used herein, an “ether bond” refers to a chemical bond comprising an oxygen bonded between two carbon atoms. In some embodiments, ether bonds link sugar residues to other entities, including, but not limited to other sugar residues to form a glycan chain. Such bonds are also referred to as “glycosidic bonds” or “glycosidic linkages”. In the context of at least one sugar residue, the terms “link” and/or “linkage” are also used herein when referring to a glycosidic linkage. In some embodiments, linkages may link glycans to other entities, including, but not limited to proteins, lipids, phospholipids and sphingolipids. In some embodiments, sugar residues may be linked to protein, typically forming a link between a sugar residue and an amino acid residue. Such amino acid residues include serine and threonine. In some embodiments, ether bonds link glycans to a glycan array comprising a carbohydrate linker that participates in bond formation. Glycosidic linkages may differ in their stereochemical properties. In some embodiments, alpha oriented glycosidic linkages (also referred to herein as “alpha linkages”) result in an axial orientation between the bonded oxygen of the ether bond and the cyclohexane ring of the sugar reside. In some embodiments, beta oriented glycosidic linkages (also referred to herein as “beta linkages”) result in an equatorial orientation between the bonded oxygen of the ether bond and the cyclohexane ring of the sugar residue.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” refers to a material or mixture prepared according to a formula and which may comprise at least one antibody, compound, substance, entity, moiety, cargo or payload and a delivery agent, carrier or excipient.

Functional: As used herein, a “functional” biological molecule is a biological entity with a structure and in a form in which it exhibits a property and/or activity by which it is characterized. As used herein, a “functional group” or “chemical group” refers to a characteristic group of atoms or chemical bonds that are part of a larger molecule. In some embodiments, functional groups may be associated with different molecules, but may participate in similar chemical reactions regardless of the molecule of which they are a part. Common functional groups include, but are not limited to carboxyl groups (—COOH), acetyl groups (—COH), amino groups (—NH₂), methyl groups (—CH₃), sulfate groups (—SO₃H) and acyl groups. In some embodiments, the addition of one or more functional group to a molecule may be conveyed using terms that modify the name of the functional group with the ending “-ylated”, e.g., acetylated, methylated and sulfated.

Glycan: As used herein, the terms “glycan”, “oligosaccharide” and “polysaccharide” are used interchangeably and refer to polymers made up of sugar monomers, typically joined by glycosidic bonds also referred to herein as linkages. In some embodiments, the terms “glycan”, “oligosaccharide” and “polysaccharide” may be used to refer to the carbohydrate portion of a glycoconjugate (e.g., glycoprotein, glycolipid or proteoglycan).

Glycan chain: As used herein, the term “glycan chain” refers to a sugar polymer comprising two or more sugars. In some embodiments, glycan chains are covalently linked to proteins through serine or threonine residues on the protein.

Glycan-rich composition: As used herein, the term “glycan-rich composition” refers to composition comprising a large percentage of glycans. In some embodiments, glycans within a glycan-rich composition may comprise from about 1% to about 10%, from about 5% to about 15%, from about 20% to about 40%, from about 30% to about 50%, from about 60% to about 80%, from about 70% to about 90% or at least 100% of the total weight of the composition.

Glycosidic bond: As used herein, the term “glycosidic bond” refers to a covalent bond formed between a carbohydrate and another chemical group. In some embodiments, glycosidic bonds are formed between the reducing end of one sugar molecule and the non-reducing end of a second sugar molecule or polysaccharide chain. Such glycosidic bonds are also known as O-glycosidic bonds due to the oxygen (or ether bond) between the joined sugars. In some embodiments, a glycosidic bond between two sugars or between a sugar and a linker may also be referred to as a “linkage”.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kit: As used herein, the term “kit” refers to a set comprising one or more components adapted for a cooperative purpose and instructions for use thereof.

Knockout: As used herein, the term “knockout” refers to an organism wherein an existing gene has been inactivated through a process that typically involves the hand of man. In a knockout organism, a gene that has been inactivated is said to have been “knocked out”. In some embodiments, the knocked out gene may be inactivated through the insertion of a nucleotide sequence into the gene or through replacement of the gene entirely.

Linker: As used herein, a “linker” refers to a moiety that connects two or more domains, moieties or entities. In one embodiment, a linker may comprise 10, 11, 12, 13, 14, 15 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent) or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris(2-carboxyethyl)phosphine (TCEP), or other reducing agents, and/or photolysis, as well as an ester bond which may be cleaved for example by acidic or basic hydrolysis. In some embodiments, a linker is a carbohydrate moiety used to link glycans to a substrate, such as in a glycan array. Such carbohydrate linkers include, but are not limited to —O(CH₂)₂CH₂HN₂ and —O(CH₂)₃NHCOCH₂ (OCH₂CH₂)₆NH₂.

mRNA: As used herein, the term “mRNA” refers to messenger RNA produced as a result of gene transcription and processing of the generated transcript. In some embodiments, mRNA that has left the nucleus of the cell may be extracted from a cell or set of cells and analyzed to determine which genes have undergone transcription at a given time or under a given set of circumstances.

Mucin: As used herein, the term “mucin” refers to a family of proteins that are heavily glycosylated. In some embodiments mucins are produced by the submaxillary glands and are found in saliva and mucous.

Negative selection: As used herein, the term “negative selection” refers to the selection of library members from a display library based on their ability to bind entities and/or components of a composition that do not comprise a target antigen. In some embodiments, negative selection is used prior to positive selection to remove elements that might bind non-specifically to the target.

Off-target: As used herein, “off target” refers to any unintended effect on any one or more target, gene, or cellular transcript.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Peptide: As used herein, “peptide” is a protein or polypeptide which is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than active agents (e.g., as described herein) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Positive selection: As used herein, the term “positive selection” refers to the selection of a given entity from a group of unique entities. Such entities and groups thereof may be, for example antibodies. In some cases they may be antibody fragments or antibody fragments expressed is association with an agent capable of expressing such fragments (e.g. library members from a display library). Selection may be based on the ability of selected entities to bind to a desired target or epitope. In some embodiments, positive selection may be used with phage display libraries to identify phage particles expressing scFvs that bind to the desired target. In other embodiments, positive selection may refer to the selection of antibody candidates from among a pool of antibodies. In other cases, entities may be cells, cell lines or clones as in the selection of clones during hybridoma selection. In such cases, positive selection may refer to clonal selection based on one or more features of antibodies (e.g. specificity for one or more desired epitopes) produced by such clones. In some cases, desired epitopes in positive selection methods may comprise STn (e.g. AcSTn and/or GcSTn).

Conversely, “negative selection,” as used herein, included the same principles and examples described for positive selection, but with the distinguishing characteristic that it is used for removal of undesired entities from a group of unique entities.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Proximal: As used herein, the term “proximal” means situated nearer to the center or to a point or region of interest.

Region of interaction: As used herein, the term “region of interaction” refers to a region along any of two or more entities where such entities interact or overlap. In some embodiments, a region of interaction may comprise one or more sugar residues along a glycan chain that contacts a second glycan chain. In some embodiments, the glycan chains are branching chains from the same parent chain. In some embodiments, a region of interaction may occur between two glycan chains wherein one chain is a branching chain and the second chain is a parent chain. In the case of glycan chains, regions of interaction may comprise 1, 2, 3, 4, 5, 6, 7, 8, 9 or at least 10 sugar residues. In some embodiments, regions of interaction may also occur between glycans and proteins or between glycans and lipids.

Residue: As used herein, the term “residue” refers to a monomer associated with or capable of associating with a polymer. In some embodiments, residues comprise sugar molecules including, but not limited to glucose, galactose, N-acetylglucosamine, N-acetylgalactosamine, sialic acids. In some embodiments, residues comprise amino acids.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Sialyl: As used herein, the prefix “sialyl” as well as the term “sialylated” describe compounds comprising sialic acid.

Single-chain variable fragment: As used herein, the term “single-chain variable fragment” or “scFv” refers to a fusion protein comprising antibody variable regions connected by a linker. In some embodiments, scFvs are utilized in conjunction with phage display methods where they may be expressed in association with a phage coat protein and used in the identification of high affinity peptides for a given antigen.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc).

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Submaxillary glands: As used herein, the term “submaxillary glands” or “submandibular glands” refers to mucous producing glands located beneath the mouth floor. These glands are capable of producing mucins and in some embodiments, may be extracted from mammals as a source of mucin.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Target: As used herein, the term “target” refers to an object or entity to be affected by an action. In some embodiments, targets refer to antigens to be used for the development of antibodies that specifically bind the antigens.

Target screening: As used herein, the term “target screening” refers to the use of a target substance to identify binding partners for that substance.

Target site: As used herein, the term “target site” refers to a target on or within one or more glycans, biomolecules and/or biostructures within a cell, the extracellular space, a tissue, an organ and/or an organism. In some embodiments, glycan target sites may reside exclusively on one sugar residue or may be formed by two or more residues. In some embodiments, target sites are formed between two or more glycans. In some embodiments, target sites are formed between branching chains of the same glycan or between one or more branching chains and a parent chain.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Terminal residue: As used herein, the term “terminal residue” refers to the last residue in a polymeric chain. In some embodiments, terminal residues are sugar residues located at the non-reducing end of a polysaccharide chain.

Therapeutic agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transgenic: As used herein, the term “transgenic” refers to an organism that comprises one or more genes incorporated within the organisms genome that are not naturally found in that organism.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Variable region: As used herein, the term “variable region” or “variable domain” refers to specific antibody domains that differ extensively in sequence among antibodies and are used in the binding and specificity of each particular antibody for its particular antigen.

Whole IgG: As used herein, the term “whole IgG” refers to a complete IgG molecule. In some embodiments, whole IgG molecules comprise regions found naturally in two or more other organisms.

Wild type: As used herein, the term “wild type” refers to an organism comprising a natural genome (free from genes derived from other organisms).

### I. COMPOSITIONS OF THE INVENTION

The present invention provides compounds as well as compositions that comprise at least one glycan-interacting antibody. As used herein, the term “glycan” refers to a polysaccharide comprising a polymeric chain of two or more monosaccharides. Within a glycan, monosaccharide monomers may all be the same or they may differ. Common monomers include, but are not limited to trioses, tetroses, pentoses, glucose, fructose, galactose, xylose, arabinose, lyxose, allose, altrose, mannose, gulose, iodose, ribose, mannoheptulose, sedoheptulose and talose. Amino sugars may also be monomers within a glycan. Glycans comprising such sugars are herein referred to as aminoglycans. Amino sugars, as used herein, are sugar molecules that comprise an amine group in place of a hydroxyl group, or in some embodiments, a sugar derived from such a sugar. Examples of amino sugars include, but are not limited to glucosamine, galactosamine, N-acetylglucosamine, N-acetylgalactosamine, sialic acids (including, but not limited to, N-acetylneuraminic acid and N-glycolylneuraminic acid) and L-daunosamine.

As used herein the term “glycan-interacting antibody” refers to an antibody that can interact with a glycan moiety. Glycan-interacting antibodies may function to bind to, alter, activate, inhibit, stabilize, degrade and/or modulate a glycan or a glycan-associated molecule or entity. In so doing, glycan-interacting antibodies may function as a therapeutic, whether palliative, prophylactic or as an ongoing treatment composition. In some embodiments, glycan-interacting antibodies may comprise conjugates or combinations with other molecules. In some embodiments, glycan-interacting antibodies are directed toward glycans comprising one or more amino sugar. In a further embodiment, one or more amino sugars is a sialic acid. In a further embodiment, one or more sialic acids is N-acetylneuraminic acid and/or N-glycolylneuraminic acid.

### Antibodies

Glycan-interacting antibodies may comprise entire antibodies or fragments thereof. As used herein, the term “antibody” is used in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications such as with sugar moieties.

“Antibody fragments” comprise a portion of an intact antibody, preferably comprising an antigen binding region thereof. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Glycan-interacting antibodies may comprise one or more of these fragments. For the purposes herein, an “antibody” may comprise a heavy and light variable domain as well as an Fc region.

“Native antibodies” are usually heterotetrameric glycoproteins of about 150,000 daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia PA. 2012. Ch. 3, p 47-54, the contents of which are herein incorporated by reference in their entirety).

VH and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. Peer J. 2:e456). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia PA. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia PA. 2012. Ch. 3, p 46-47, the contents of which are herein incorporated by reference in their entirety).

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2a, IgG2b, IgG2c, IgG3, IgG4, IgA, and IgA2.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding.

The term “diabodies” refers to small antibody fragments with two antigen-binding sites, which fragments comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), the contents of each of which are incorporated herein by reference in their entirety.

The term “intrabody” refers to a form of antibody that is not secreted from a cell in which it is produced, but instead target one or more intracellular protein. Intrabodies may be used to affect a multitude of cellular processes including, but not limited to intracellular trafficking, transcription, translation, metabolic processes, proliferative signaling and cell division. In some embodiments, methods of the present invention may include intrabody-based therapies. In some such embodiments, variable domain sequences and/or CDR sequences disclosed herein may be incorporated into one or more construct for intrabody-based therapy. In some cases, intrabodies of the invention may target one or more glycated intracellular protein or may modulate the interaction between one or more glycated intracellular protein and an alternative protein.

The term “chimeric antigen receptor” or “CAR” as used herein, refers to an artificially constructed hybrid protein or polypeptide receptor (also known as a “chimeric immunoreceptor,” “artificial T cell receptor” or “chimeric T cell receptor”) containing the antigen binding domains of an antibody (scFv) linked to T-cell signaling domains. CARs are engineered to be expressed on the surface of immune effector cells, thereby allowing the immune effector cells to specifically target other cells (such as tumor cells) that express the corresponding antigenic entity via a high affinity interaction between the target cell and the immune effector cell bearing the CAR. CARs can be designed to specifically bind cancer cells, leading to immune-regulated clearance of the cancer cells. The phrases “have antigen specificity” and “elicit antigen-specific response” as used with respect to CARs means that the CAR can specifically bind to and immunologically recognize an antigen to elicit an immune response.

The term “monoclonal antibody” as used herein refers to an antibody obtained from a population of substantially homogeneous cells (or clones), i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

In some embodiments, glycan-interacting antibodies of the present invention may be antibody mimetics. The term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure and/or function comprising some differences in their amino acid sequence, composition or structure as compared to a native antibody.

### Antibody Development

Glycan-interacting antibodies of the present invention are developed to bind antigens such as those described herein. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. In some cases, methods of immunization may be altered based on one or more desired immunization outcomes. As used here, the term “immunization outcome” refers to one or more desired effects of immunization. Examples include high antibody titers and/or increased antibody specificity for a target of interest.

Antigens of the invention may comprise glycans, glycoconjugates (including, but not limited to glycoproteins and glycolipids), peptides, polypeptides, fusion proteins, or any of the foregoing and may be conjugated or complexed to one or more separate adjuvants or heterologous proteins. In some embodiments, antigens used according to methods of the present invention may comprise sialylated glycans, such as STn. Antigens comprising STn may comprise mucins. Mucins are a family of proteins that are heavily glycosylated. They are a component of many tumors originating from epithelial cells (Ishida, A. et al., 2008. Proteomics. 8: 3342-9, the contents of which are herein incorporated by reference in their entirety). They are highly expressed by submaxillary glands and can be found at high levels in saliva and mucous. Animal-derived submaxillary mucins may be used as antigens to generate anti-STn antibodies in immunogenic hosts. Submaxillary mucin from different species differ in their STn content with regard to AcSTn versus GcSTn forms. Porcine submaxillary mucin (PSM) is particularly rich in GcSTn, which makes up about 90% of total STn. STn from bovine submaxillary mucin (BSM) comprises roughly equal percentages of GcSTn and AcSTn. Ovine submaxillary mucin (OSM) is particularly rich in AcSTn, which makes up about 90% of total STn. In some cases, solutions prepared for immunization may be modified to include one or more of PSM, BSM and OSM depending on the desired target of antibodies resulting from such immunization. PSM may be used in immunizations to generate antibodies in immunogenic hosts that are more likely to be specific for GcSTn. PSM is rich in Neu5Gc-containing mucin-type, glycoproteins that are decorated with GcSTn. Among the currently known sources of high Neu5Gc content is red meat; especially submaxillary glands were previously described as a rich source of Neu5Gc due to the high expression of the CMAH enzyme, which catalyzes the reaction to produce the Neu5Gc precursor, CMP-Neu5Ac. In some cases, PSM may be used to prevent a pan-anti-Neu5Gc response and induce a more specific immune response against GcSTn. OSM may be used in immunizations to generate antibodies in immunogenic hosts that are more likely to be specific for AcSTn.

In one embodiment, the present invention provides a glycan-interacting antibody that is GcSTn-specific. The antibody has little cross-reactivity to Neu5Ac-STn or Tn. The antibody can bind GcSTn but has reduced affinity for AcSTn.

In some embodiments, antigens may be subjected to enzymatic digestion prior to immunization to modulate the resulting immune response in immunogenic hosts. In one example, submaxillary mucins may be treated with trypsin or proteinase K enzymes prior to immunization. The activity of such enzymes may help to cleave off and thereby reduce the percentage and variability of non-STn epitopes. Glycan moieties may shield regions of the peptide where they are attached from enzymatic proteolysis and thereby remain intact. Antibody titers resulting from immunizations may comprise different levels depending on the type and amount of antigen used in such immunizations. In some cases, certain antigens may be selected for use in immunizations based on the expected titer.

As used herein, an “adjuvant” is a pharmacological or immunological agent that modifies the effect of other agents. Adjuvants according to the present invention include, but are not limited chemical compositions, biomolecules, therapeutics, and/or therapeutic regimens. Adjuvants may include Freund's adjuvant (complete and/or incomplete), immunostimulatory oligonucleotides [e.g. CpG oligodeoxynucleotides (ODNs)], mineral-containing compositions, bacterial ADP-ribosylating toxins, bioadhesives, mucoadhesives, microparticles, lipids, liposomes, muramyl peptides, N-oxidized polyethylene-piperazine derivatives, saponins and/or immune stimulating complexes (ISCOs). In some embodiments, adjuvants may comprise oil-in-water emulsions (e.g. sub-micron oil-in-water emulsions). Adjuvants according to the present invention may also include any of those disclosed in US Patent Publication No. US20120027813 and/or U.S. Pat. No. 8,506,966, the contents of each of which are herein incorporated by reference in their entirety.

Antibodies of the present invention may be polyclonal or monoclonal or recombinant, produced by methods known in the art or as described in this application. In some embodiments, the antibodies of the present invention may be labeled for purposes of detection with a detectable label known by one of skill in the art. The label can be a radioisotope, fluorescent compound, chemiluminescent compound, enzyme, or enzyme co-factor, or any other labels known in the art. In some aspects, the antibody that binds to a desired antigen is not labeled, but may be detected by binding of a labeled secondary antibody that specifically binds to the primary antibody.

Antibodies of the present invention (e.g., glycan-interacting antibodies) include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. Antibodies of the present invention (e.g., glycan-interacting antibodies) can be from any animal origin including birds and mammals. Preferably, such antibodies are of human, murine (e.g., mouse and rat), donkey, sheep, rabbit, goat, guinea pig, camel, horse, or chicken origin. The antibodies of the present invention can be monospecific or multispecific (e.g., bispecific, trispecific, or of greater multispecificity). Multispecific antibodies can be specific for different epitopes of a target antigen of the present invention, or can be specific for both a target antigen of the present invention, and a heterologous epitope, such as a heterologous glycan, peptide or solid support material. (See, e.g., WO 93/17715; WO 92/08802; WO 91/00360; WO 92/05793; Tutt, A. et al., Trispecific F(ab)3 derivatives that use cooperative signaling via the TCR/CD3 complex and CD2 to activate and redirect resting cytotoxic T cells. J Immunol. 1991 Jul. 1; 147(1):60-9; U.S. Pat. Nos. 4,474,893; 4,714,681; 4,925,648; 5,573,920; 5,601,819; and Kostelny, S. A. et al., Formation of a bispecific antibody by the use of leucine zippers. J Immunol. 1992 Mar. 1; 148(5):1547-53).

Glycan-interacting antibodies of the present invention comprising monoclonal antibodies can be prepared using well-established methods known by those skilled in the art. In one embodiment, the monoclonal antibodies are prepared using hybridoma technology (Kohler, G. et al., Continuous cultures of fused cells secreting antibody of predefined specificity. Nature. 1975 Aug. 7; 256(5517):495-7). For hybridoma formations, first, a mouse, hamster, or other appropriate host animal, is typically immunized with an immunizing agent (e.g., a target antigen of the invention) to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, J. W., Monoclonal Antibodies: Principles and Practice. Academic Press. 1986; 59-1031). Immortalized cell lines are usually transformed mammalian cells, particularly myeloma cells of rodent, rabbit, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, D. et al., A human hybrid myeloma for production of human monoclonal antibodies. J Immunol. 1984 December; 133(6):3001-5; Brodeur, B. et al., Monoclonal Antibody Production Techniques and Applications. Marcel Dekker, Inc., New York. 1987; 33:51-63).

In some embodiments, myeloma cells may be subjected to genetic manipulation. Such manipulation may be carried out using zinc-finger nuclease (ZFN) mutagenesis as described herein. Alternatively, transfection methods known in the art may be used. NS0 myeloma cells or other mouse myeloma cell lines may be used. For example, Sp2/0-Ag14 can be an alternative cell line for hybridoma development.

Transcription Activator-Like Effector Nucleases (TALENs)-induced gene editing provides an alternative gene knock out method. TALENs are artificial restriction enzymes generated by fusing the TAL effector DNA binding domain to a DNA cleavage domain. Similar to ZFNs, TALENs induce double-strand breaks at desired loci that can be repaired by error-prone NHEJ to yield insertions/deletions at the break sites (Wood, A. J. et al., Targeted genome editing across species using ZFNs and TALENs. Science. 2011 Jul. 15; 333(6040):307). Cellectis Bioresearch (Cambridge, MA) provides the service of TALEN design and plasmid construction. The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known by those skilled in the art. The binding specificity of the monoclonal antibody can, for example, be determined by Scatchard analysis (Munson, P. J. et al., Ligand: a versatile computerized approach for characterization of ligand-binding systems. Anal Biochem. 1980 Sep. 1; 107(1):220-39). In some cases, antibody specificity for regions of a given antigen may be characterized by chemically modifying the antigens prior to assaying for antibody binding. In one example, periodate treatment may be used to to destroy the C6 side chain of sialic acids. Assays may be conducted with and without periodate treatment to reveal whether or not binding in untreated samples is sialic acid-specific. In some cases, antigens comprising 9-O-acetylated sialic acid may be subjected to mild base treatment (e.g. with 0.1 M NaOH) to destroy 9-O-acetyl groups. Assays may be conducted with and without mild base treatment to reveal whether or not binding in untreated samples depends on 9-O-acetylation of sialic acid.

After the desired hybridoma cells are identified, the clones may be subcloned by limiting dilution procedures and grown by standard methods. Suitable culture media for this purpose include, for example, Dulbecco's Modified Eagle's Medium or RPMI-1640 medium. Alternatively, the hybridoma cells may be grown in vivo as ascites in a mammal.

Alternative methods to clone hybridomas may include those provided by kits from STEMCELL Technologies (Vancouver, BC, Canada), e.g. ClonaCell™-HY kit, containing methylcellulose-based semi-solid medium and other media and reagents, to support the selection and growth of hybridoma clones. However, the media in this kit contain FCS, which provides an exogenous source for Neu5Gc incorporation. Though the machinery for endogenous Neu5Gc synthesis is destroyed in Cmah^{-/-} hybridoma, Neu5Gc incorporated from the culture media may also pose a problem in some cases (Bardor, M. et al., Mechanism of uptake and incorporation of the non-human sialic acid N-glycolylneuraminic acid into human cells. J Biol Chem. 2005. 280: 4228-4237). In such instances, The culture media may be supplemented with Neu5Ac to eliminate Neu5Gc incorporation by metabolic competition (Ghaderi, D. et al., Implications of the presence of N-glycolylneuraminic acid in recombinant therapeutic glycoproteins. Nat Biotechnol. 2010. 28: 863-867).

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In another embodiment, the monoclonal antibodies of the present invention can also be made by recombinant DNA methods, such as those described in U.S. Pat. No. 4,816,567, which is hereby incorporated by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiments, antibodies of the present invention (e.g., glycan-interacting antibodies) may be produced by various procedures known by those skilled in the art. For the production of polyclonal antibodies in vivo, host animals, such as rabbits, rats, mice, cows, horses, donkeys, chickens, monkeys, sheep or goats, are immunized with either free or carrier-coupled antigens, for example, by intraperitoneal and/or intradermal injection. In some embodiments, injection material may be an emulsion containing about 100 μg of antigen or carrier protein. In some embodiments, injection materials comprise a glycan-rich composition such as non-human mammalian submaxillary mucin in solution. Various adjuvants can also be used to increase the immunological response, depending on the host species. Adjuvants include, but are not limited to, Freund's (complete and incomplete), mineral gels such as aluminum hydroxide, surface active substances such as lysolecithin, pluronic polyols, polyanions, peptides, oil emulsions, TITERMAX® (CytRx Corp, Los Angeles, CA), keyhole limpet hemocyanins, dinitrophenol, and potentially useful human adjuvants such as BCG (bacille Calmette-Guerin) and Corynebacterium parvum. Such adjuvants are also well known in the art. Several booster injections may be needed, for instance, at intervals of about two weeks, to provide a useful titer of antibody which can be detected, for example, by ELISA assay using glycans and/or free peptide adsorbed to a solid surface. The titer of antibodies in serum from an immunized animal can be increased by selection of antibodies, e.g., by adsorption of antigens onto a solid support and elution of the selected antibodies according to methods well known in the art.

Glycan-interacting antibodies, variants and fragments thereof may be selected and produced using high throughput methods of discovery. In one embodiment, glycan-interacting antibodies comprising synthetic antibodies, variants and fragments thereof are produced through the use of display libraries. The term “display” as used herein, refers to the expression or “display” of proteins or peptides on the surface of a given host. The term “library” as used herein, refers to a collection of unique cDNA sequences and/or the proteins that are encoded by them. A library may contain from as little as two unique cDNAs to hundreds of billions of unique cDNAs. In a preferred embodiment, glycan-interacting antibodies comprising synthetic antibodies are produced using antibody display libraries or antibody fragment display libraries. The term “antibody fragment display library” as used herein, refers to a display library wherein each member encodes an antibody fragment containing at least one variable region of an antibody. Such antibody fragments are preferably Fab fragments, but other antibody fragments such as single-chain variable fragments (scFvs) are contemplated as well. In an Fab antibody fragment library, each Fab encoded may be identical except for the amino acid sequence contained within the variable loops of the complementarity determining regions (CDRs) of the Fab fragment. In an alternative or additional embodiment, amino acid sequences within the individual V_{H} and/or V_{L} regions may differ as well.

Display libraries may be expressed in a number of possible hosts including, but not limited to yeast, bacteriophage, bacteria and retroviruses. Additional display technologies that may be used include ribosome-display, microbead-display and protein-DNA linkage techniques. In a preferred embodiment, Fab display libraries are expressed in yeast or in bacteriophages (also referred to herein as “phages” or “phage particles”. When expressed, the Fabs decorate the surface of the phage or yeast where they can interact with a given antigen. An antigen comprising a glycan or other antigen from a desired target may be used to select phage particles or yeast cells expressing antibody fragments with the highest affinity for that antigen. The DNA sequence encoding the CDR of the bound antibody fragment can then be determined through sequencing using the bound particle or cell. In one embodiment, positive selection is used in the development of antibodies. In some embodiments, negative selection is utilized in the development of antibodies. In some embodiments, both positive and negative selection methods are utilized during multiple rounds of selection in the development of antibodies using display libraries.

In yeast display, cDNA encoding different antibody fragments are introduced into yeast cells where they are expressed and the antibody fragments are “displayed” on the cell surface as described by Chao et al. (Chao, G. et al., Isolating and engineering human antibodies using yeast surface display. Nat Protoc. 2006; 1(2):755-68). In yeast surface display, expressed antibody fragments contain an additional domain comprising the yeast agglutinin protein, Aga2p. This domain allows the antibody fragment fusion protein to attach to the outer surface of the yeast cell through the formation of disulphide bonds with surface-expressed Aga1p. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired antigenic target antigen, incubated with the cells. Target antigens may be covalently or otherwise modified with a chemical or magnetic group to allow for efficient cell sorting after successful binding with a suitable antibody fragment takes place. Recovery may be by way of magnetic-activated cell sorting (MACS), fluorescence-activated cell sorting (FACS) or other cell sorting methods known in the art. Once a subpopulation of yeast cells is selected, the corresponding plasmids may be analyzed to determine the CDR sequence.

Bacteriophage display technology typically utilizes filamentous phage including, but not limited to fd, F1 and M13 virions. Such strains are non-lytic, allowing for continued propagation of the host and increased viral titres. Examples of phage display methods that can be used to make the antibodies of the present invention include those disclosed in Miersch et al. (Miersch, S. et al., Synthetic antibodies: Concepts, potential and practical considerations. Methods. 2012 August; 57(4):486-98), Bradbury et al. (Bradbury, A. R. et al., Beyond natural antibodies: the power of in vitro display technologies. Nat Biotechnol. 2011 March; 29(3):245-54), Brinkman et al. (Brinkmann, U. et al., Phage display of disulfide-stabilized Fv fragments. J Immunol Methods. 1995 May 11; 182(1):41-50); Ames et al. (Ames, R. S. et al., Conversion of murine Fabs isolated from a combinatorial phage display library to full length immunoglobulins. J Immunol Methods. 1995 Aug. 18; 184(2):177-86); Kettleborough et al. (Kettleborough, C. A. et al., Isolation of tumor cell-specific single-chain Fv from immunized mice using phage-antibody libraries and the re-construction of whole antibodies from these antibody fragments. Eur J Immunol. 1994 April; 24(4):952-8); Persic et al. (Persic, L. et al., An integrated vector system for the eukaryotic expression of antibodies or their fragments after selection from phage display libraries. Gene. 1997 Mar. 10; 187(1):9-18); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5, 969,108, each of which is incorporated herein by reference in its entirety. Antibody fragment expression on bacteriophages may be carried out by inserting the cDNA encoding the fragment into the gene expressing a viral coat protein. The viral coat of filamentous bacteriophages is made up of five coat proteins, encoded by a single-stranded genome. Coat protein pIII is the preferred protein for antibody fragment expression, typically at the N-terminus. If antibody fragment expression compromises the function of pIII, viral function may be restored through coexpression of a wild type pIII, although such expression will reduce the number of antibody fragments expressed on the viral coat, but may enhance access to the antibody fragment by the target antigen. Expression of viral as well as antibody fragment proteins may alternatively be encoded on multiple plasmids. This method may be used to reduce the overall size of infective plasmids and enhance the transformation efficiency.

As described above, after selection of a host expressing a high affinity antibody or antibody fragment, (e.g., glycan-interacting antibodies) the coding regions from the antibody or antibody fragment can be isolated and used to generate whole antibodies, including human antibodies, or any other desired antigen binding fragment, and expressed in any desired host, including mammalian cells, insect cells, plant cells, yeast, and bacteria, e.g., as described in detail below.

The DNA sequence encoding a high affinity antibody can be mutated for additional rounds of selection in a process known as affinity maturation. The term “affinity maturation”, as used herein, refers to a method whereby antibodies are produced with increasing affinity for a given antigen through successive rounds of mutation and selection of antibody- or antibody fragment-encoding cDNA sequences. In a preferred embodiment, this process is carried out in vitro. To accomplish this, amplification of CDR coding sequences may be carried out using error-prone PCR to produce millions of copies containing mutations including, but not limited to point mutations, regional mutations, insertional mutations and deletional mutations. As used herein, the term “point mutation” refers to a nucleic acid mutation in which one nucleotide within a nucleotide sequence is changed to a different nucleotide. As used herein, the term “regional mutation” refers to a nucleic acid mutation in which two or more consecutive nucleotides are changed to different nucleotides. As used herein, the term “insertional mutation” refers to a nucleic acid mutation in which one or more nucleotides are inserted into a nucleotide sequence. As used herein, the term “deletional mutation” refers to a nucleic acid mutation in which one or more nucleotides are removed from a nucleotide sequence. Insertional or deletional mutations may include the complete replacement of an entire codon or the change of one codon to another by altering one or two nucleotides of the starting codon.

Mutagenesis may be carried out on CDR-encoding cDNA sequences to create millions of mutants with singular mutations in CDR heavy and light chain regions. In another approach, random mutations are introduced only at CDR residues most likely to improve affinity. These newly generated mutagenic libraries can be used to repeat the process to screen for clones that encode antibody fragments with even higher affinity for the target antigen. Continued rounds of mutation and selection promote the synthesis of clones with greater and greater affinity (Chao, G. et al., Isolating and engineering human antibodies using yeast surface display. Nat Protoc. 2006; 1(2):755-68).

Examples of techniques that can be used to produce antibodies and antibody fragments, such as Fabs and scFvs, include those described in U.S. Pat. Nos. 4,946,778 and 5,258,498; Miersch et al. (Miersch, S. et al., Synthetic antibodies: Concepts, potential and practical considerations. Methods. 2012 August; 57(4):486-98), Chao et al. (Chao, G. et al., Isolating and engineering human antibodies using yeast surface display. Nat Protoc. 2006; 1(2):755-68), Huston et al. (Huston, J. S. et al., Protein engineering of single-chain Fv analogs and fusion proteins. Methods Enzymol. 1991; 203:46-88); Shu et al. (Shu, L. et al., Secretion of a single-gene-encoded immunoglobulin from myeloma cells. Proc Natl Acad Sci USA. 1993 Sep. 1; 90(17):7995-9); and Skerra et al. (Skerra, A. et al., Assembly of a functional immunoglobulin Fv fragment in Escherichia coli. Science. 1988 May 20; 240(4855):1038-41), each of which is incorporated herein by reference in its entirety.

For some uses, including the in vivo use of antibodies (e.g., glycan-interacting antibodies) in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are known in the art. (Morrison, S. L., Transfectomas provide novel chimeric antibodies. Science. 1985 Sep. 20; 229(4719):1202-7; Gillies, S. D. et al., High-level expression of chimeric antibodies using adapted cDNA variable region cassettes. J Immunol Methods. 1989 Dec. 20; 125(1-2):191-202.; and U.S. Pat. Nos. 5,807,715; 4,816,567; and 4,816,397, which are incorporated herein by reference in their entirety).

Humanized antibodies are antibody molecules from non-human species that bind to the desired antigen and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (U.S. Pat. Nos. 5,693,762 and 5,585,089; Riechmann, L. et al., Reshaping human antibodies for therapy. Nature. 1988 Mar. 24; 332(6162):323-7, which are incorporated herein by reference in their entireties). Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, E. A., A possible procedure for reducing the immunogenicity of antibody variable domains while preserving their ligand-binding properties. Mol Immunol. 1991 April-May; 28(4-5):489-98; Studnicka, G. M. et al., Human-engineered monoclonal antibodies retain full specific binding activity by preserving non-CDR complementarity-modulating residues. Protein Eng. 1994 June; 7(6):805-14; Roguska, M. A. et al., Humanization of murine monoclonal antibodies through variable domain resurfacing. Proc Natl Acad Sci USA. 1994 Feb. 1; 91(3):969-73); and chain shuffling (U.S. Pat. No. 5,565,332); each of which is incorporated herein by reference in their entirety. Humanized antibodies of the present invention may be developed for desired binding specificity, complement-dependent cytotoxicity, and antibody-dependent cellular-mediated cytotoxicity, etc.

Completely human antibodies (e.g., glycan-interacting antibodies) are particularly desirable for therapeutic treatment of human patients, so as to avoid or alleviate immune reaction to foreign protein. Human antibodies can be made by a variety of methods known in the art, including the antibody display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies (e.g., glycan-interacting antibodies) can also be produced using transgenic mice which are incapable of expressing functional endogenous immunoglobulins, but which can express human immunoglobulin polynucleotides. For example, the human heavy and light chain immunoglobulin polynucleotide complexes can be introduced randomly, or by homologous recombination, into mouse embryonic stem cells. Alternatively, the human variable region, constant region, and diversity region may be introduced into mouse embryonic stem cells, in addition to the human heavy and light chain polynucleotides. The mouse heavy and light chain immunoglobulin polynucleotides can be rendered nonfunctional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a glycan, glycoconjugate and/or polypeptide of the invention.

Thus, using such a technique, it is possible to produce useful human IgG, IgA, IgM, IgD and IgE antibodies. For an overview of the technology for producing human antibodies, see Lonberg and Huszar (Lonberg, N. et al., Human antibodies from transgenic mice. Int Rev Immunol. 1995; 13(1):65-93). For a detailed discussion of the technology for producing human antibodies and human monoclonal antibodies and protocols for producing such antibodies, see, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, each of which are incorporated by reference herein in their entirety. In addition, companies such as Abgenix, Inc. (Fremont, Calif.), Protein Design Labs, Inc. (Mountain View, Calif.) and Genpharm (San Jose, Calif.) can be engaged to provide human antibodies directed against a selected antigen using technology similar to the above described technologies.

Once an antibody molecule of the present invention has been produced by an animal, a cell line, chemically synthesized, or recombinantly expressed, it can be purified (i.e., isolated) by any method known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The affinity between an antibody and a target or ligand (such as an antigen used to generate a given antibody) may be measured in terms of K_{D} using one or more binding assays as described herein. Depending on the desired application for a given antibody, varying KD values may be desirable. High affinity antibodies typically form ligand bonds with a KD of about 10⁻⁵ M or less, e.g. about 10⁻⁶ M or less, about 10⁻⁷ M or less, about 10⁻⁸ M or less, about 10⁻⁹ M or less, about 10⁻¹⁰ M or less, about 10⁻¹¹ M or less or about 10⁻¹² M or less.

In some embodiments, antibodies of the invention may be characterized according to their half maximal effective or inhibitory concentration (EC₅₀ or IC₅₀, respectively). In some cases, this value may represent the concentration of antibody necessary to inhibit cells expressing STn (e.g. kill and/or reduce proliferation) at a level equal to half of the maximum inhibition observed with the highest concentrations of antibody. Such IC₅₀ values may be from about 0.001 nM to about 0.01 nM, from about 0.005 nM to about 0.05 nM, from about 0.01 nM to about 1 nM, from about 0.05 nM to about 5 nM, from about 0.1 nM to about 10 nM, from about 0.5 nM to about 25 nM, from about 1 nM to about 50 nM, from about 5 nM to about 75 nM, from about 10 nM to about 100 nM, from about 25 nM to about 250 nM, from about 200 nM to about 1000 nM or more than 1000 nM.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

### Targets

Glycan-interacting antibodies of the present invention exert their effects via binding (reversibly or irreversibly) to one or more glycan or glycan-associated or glycan-related targets. In some embodiments, glycan-interacting antibodies can be prepared from any region of the targets taught herein. In some embodiments, targets of the present invention comprise glycans. Glycans used for generating antibodies may comprise a chain of sugars comprising at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 residues. Preferably, glycans used for generating antibodies comprise from about 2 residue to about 5 residues.

In some embodiments, glycan-interacting antibody target antigens comprise sialic acids. N-acetylneuraminic acid (Neu5Ac) and N-glycolylneuraminic acid (Neu5Gc) are the major sialic acids on mammalian cell surfaces. Of these, Neu5Ac is naturally produced in humans. Neu5Gc is naturally produced in most mammals with the exception of humans due to a mutation in the cytidine monophosphate (CMP)-N-acetylneuraminic acid hydroxylase (CMAH) gene responsible for CMP-Neu5Gc production from CMP-Neu5Ac. Neu5Gc in humans is in fact immunogenic with nearly all humans expressing anti-Neu5Gc antibodies. Despite a lack of production, most human systems comprise some level of Neu5Gc due to dietary intake. These foreign products are subsequently incorporated into human glycoproteins. Such glycoproteins are contemplated as targets of the invention. Glycan target antigens of the present invention, include, but are not limited to those listed in Table 1.

TABLE 1
Glycan target antigens
Glycan

GalNAcα-R
Galα1,3Galβ1,4GlcNAcβ-R
Galβ1,3GalNAcβ-R
Galβ1,3GlcNAcα-R
Galβ1,3GlcNAcβ1,3Galβ1,4Glcβ-R
Galβ1,3GlcNAcβ-R
Galβ1,4GlcNAc6Sβ-R
Galβ1,4GlcNAcβ-R
Galβ1,4Glcβ-R
KDNα2,8Neu5Acα2,3Galβ1,4Glcβ-R
KDNα2,8Neu5Gcα2,3Galβ1,4Glcβ-R
Neu5,9Ac2α2,3Galβ1,3GalNAcα-R
Neu5,9Ac2α2,3Galβ1,3GalNAcβ-R
Neu5,9Ac2α2,3Galβ1,3GlcNAcβ-R
Neu5,9Ac2α2,3Galβ1,4GlcNAcβ-R
Neu5,9Ac2α2,3Galβ1,4Glcβ-R
Neu5,9Ac2α2,3Galβ-R
Neu5,9Ac2α2,6GalNAcα-R
Neu5,9Ac2α2,6Galβ1,4GlcNAcβ-R
Neu5,9Ac2α2,6Galβ1,4Glcβ-R
Neu5,9Ac2α2,6Galβ-R
Neu5Acα2,3Galβ1,3GalNAcα-R
Neu5Acα2,3Galβ1,3GalNAcβ-R
Neu5Acα2,3Galβ1,3GlcNAcβ1,3Galβ1,4Glcβ-R
Neu5Acα2,3Galβ1,3GlcNAcβ-R
Neu5Acα2,3Galβ1,4(Fucα1,3)GlcNAc6Sβ-R
Neu5Acα2,3Galβ1,4(Fucα1,3)GlcNAcβ-R
Neu5Acα2,3Galβ1,4GlcNAc6Sβ-R
Neu5Acα2,3Galβ1,4GlcNAcβ-R
Neu5Acα2,3Galβ1,4Glcβ-R
Neu5Acα2,3Galβ-R
Neu5Acα2,6(KDNα2,3)Galβ1,4Glcβ-R
Neu5Acα2,6(Neu5Acα2,3)Galβ1,4Glcβ-R
Neu5Acα2,6(Neu5Gcα2,3)Galβ1,4Glcβ-R
Neu5Acα2,6GalNAcα-R
Neu5Acα2,6Galβ1,4GlcNAcβ-R
Neu5Acα2,6Galβ1,4Glcβ-R
Neu5Acα2,6Galβ-R
Neu5Acα2,8KDNα2,6Galβ1,4Glcβ-R
Neu5Acα2,8Neu5Acα2,3Galβ1,4Glcβ-R
Neu5Acα2,8Neu5Acα2,3Galβ1,4Glcβ-R
Neu5Acα2,8Neu5Acα2,6Galβ1,4Glcβ-R
Neu5Acα2,8Neu5Acα2,8Neu5Acα2,3Galβ1,4Glcβ-R
Neu5Acα2,8Neu5Acα2,8Neu5Acα2,3Galβ1,4Glcβ-R
Neu5Acα2,8Neu5Gcα2,3Galβ1,4Glcβ-R
Neu5Acα2,8Neu5Gcα2,6Galβ1,4Glcβ-R
Neu5Gc9Acα2,3Galβ1,4Glcβ-R
Neu5Gc9Acα2,6Galβ1,4Glcβ-R
Neu5Gc9Acα2,3Galβ1,3GalNAcα-R
Neu5Gc9Acα2,3Galβ1,3GalNAcβ-R
Neu5Gc9Acα2,3Galβ1,3GlcNAcβ-R
Neu5Gc9Acα2,3Galβ1,4GlcNAcβ-R
Neu5Gc9Acα2,3Galβ-R
Neu5Gc9Acα2,6GalNAcα-R
Neu5Gc9Acα2,6Galβ1,4GlcNAcβ-R
Neu5Gc9Acα2,6Galβ-R
Neu5GcOMeα2,8Neu5Acα2,3Galβ1,4Glcβ-R
Neu5Gcα2,3Galβ1,3GalNAcα-R
Neu5Gcα2,3Galβ1,3GalNAcβ-R
Neu5Gcα2,3Galβ1,3GlcNAcβ1,3Galβ1,4Glcβ-R
Neu5Gcα2,3Galβ1,3GlcNAcβ-R
Neu5Gcα2,3Galβ1,4(Fucα1,3)GlcNAc6Sβ-R
Neu5Gcα2,3Galβ1,4(Fucα1,3)GlcNAcβ-R
Neu5Gcα2,3Galβ1,4GlcNAc6Sβ-R
Neu5Gcα2,3Galβ1,4GlcNAcβ-R
Neu5Gcα2,3Galβ1,4Glcβ-R
Neu5Gcα2,3Galβ-R
Neu5Gcα2,6GalNAcα-R
Neu5Gcα2,6Galβ1,4GlcNAcβ-R
Neu5Gcα2,6Galβ1,4Glcβ-R
Neu5Gcα2,6Galβ-R
Neu5Gcα2,8Neu5Acα2,3Galβ1,4Glcβ-R
Neu5Gcα2,8Neu5Gcα2,3Galβ1,4Glcβ-R

The following abbreviations are used herein: Glc—glucose, Gal—galactose, GlcNAc—N-acetylglucosamine, GalNAc—N-acetylgalactosamine, GlcNAc6S—6-Sulfo-N-acetylglucosamine, KDN—2-keto-3-deoxy-D-glycero-D-galactonononic acid, Neu5,9Ac2—N-acetyl-9-O-acetylneuraminic acid, Fuc—fucose and Neu5GcOMe—2-O-methyl-N-glycolylneuraminic acid. O-glycosidic bonds are present between each residue in the glycans listed with α and β indicating the relative stoichiometry between the two residues joined by the bond, wherein a indicates an axial orientation and R indicates an equatorial orientation. The numbers following α and/or β, in the format x,x, indicated the carbon number of each of the carbons from each of the adjoined residues that participate in bond formation. While the glycans listed in Table 1 represent individual glycan target antigens contemplated, the present invention also includes embodiments wherein the above presented glycans comprise different combinations of α and β-oriented O-glycosidic bonds than the ones presented. Also in Table 1, R represents an entity that the glycan may be coupled with. In some embodiments, R is a protein wherein the glycan is linked typically to a serine or threonine residue. In some embodiments, R is a linker molecule used to join the glycan to a substrate, such as in a glycan array. In some embodiments, R may be a linker comprising —(CH₂)₂CH₂NH₂ or —(CH₂)₃NHCOCH₂(OCH₂CH₂)₆NH₂. In some embodiments, R may be biotin, albumin, ProNH₂, —CH—, —OH, —OCH₃, —OCH₂CH₃, —H, hydrido, hydroxy, alkoxyl, oxygen, carbon, sulfur, nitrogen, polyacrylamide, phosphorus, NH₂, ProNH₂═O(CH₂)₂CH₂NH₂, (OCH₂CH₂)₆NH₂, O(CH₂)₃NHCOCH₂ (OCH₂CH₂)₆NH₂, the fluorescent labels 2-aminobenzamide (AB) and/or 2-aminobenzoid acid (AA), 2-aminobenzamide analog that contains an alkyl amine (AEAB), aminooxy-groups, methylaminooxygroups, hydrazide groups, amino lipid 1,2-dihexadecyl-sn-glycero-3-phosphoethanolamine (DHPE), aminooxy (AO) functionalized DHPE and glycosylphosphatidylinositol (GPI). Without intending to limit the source or nature of R, this may include structures that affect the physical spacing of glycan residue. In some embodiments, the R group may comprise a combination of the R groups presented here, e.g. a biotinylated polyacrylamide. In some embodiments, the R group in combination with underlying substrates effect glycan residue spacing.

Glycan targets of the present invention may comprise regions of antibody recognition. As used herein, the term “region of antibody recognition” refers to one or more regions located on any part of the molecule, an attached group or located on a region of interaction between the glycan and another molecule, including, but not limited to another glycan. In some embodiments, regions of antibody recognition are located at interchain target sites, wherein the term interchain means within the present polymeric chain. Interchain target sites may comprise regions of antibody recognition comprising 1, 2, 3, 4, 5, 6, 7, 8, 9 or at least 10 residues, bonds between residues or combinations of residues and bonds. In some embodiments, regions of antibody recognition are located at regions of interaction between one or more glycan chains. Such regions may be between 2, 3, 4 or at least 5 glycan chains.

In some embodiments, regions of antibody recognition are located at regions of interaction between glycan branch chains connected to a common parent chain. In some embodiments, regions of antibody recognition are located at regions of interaction between a glycan branch chain and a parent chain. In some embodiments, regions of antibody recognition are located at regions of interaction between glycans and proteins. Such regions of interaction may comprise chemical bonds between the glycan and the protein, including, but not limited to covalent bonds, ionic bonds, hydrostatic bonds, hydrophobic bonds and hydrogen bonds. In some embodiments, regions of antibody recognition are located at regions of interaction between glycans and other biomolecules including, but not limited to lipids and nucleic acids. Such regions of interaction may comprise chemical bonds between the glycan and the biomolecule, including, but not limited to covalent bonds, ionic bonds, hydrostatic bonds, hydrophobic bonds and hydrogen bonds.

In some embodiments, glycan targets of the present invention are components of glycoconjugates. As used herein, the term “glycoconjugate” refers to any entity comprising a glycan moiety. In some embodiments, glycoconjugates are glycolipids. As used herein, the term “glycolipid” refers to a class of lipids wherein a carbohydrate moiety is covalently attached. In some embodiments, carbohydrate moieties present on glycolipids comprise glycans. In some embodiments, lipid components of glycolipids comprise ceramide moieties. Examples of glycolipids contemplated as targets of the present invention include, but are not limited to glyceroglycolipids (including, but not limited to galactolipids and sulfolipids), glycosphingolipids (including, but not limited to cerebrosides (e.g., galactocerebrosides, glucocerebrosides and sulfatides), gangliosides, globosides and glycophosphosphingolipids) and glycosylphosphatidylinositols. When located within cell membranes, glycan moieties of glycolipids are located on the extracellular side of the membrane where they may interact with other cells as well as cell signaling ligands (Maccioni, H. J. et al., Organization of the synthesis of glycolipid oligosaccharides in the Golgi complex. FEBS Lett. 2011 Jun. 6; 585(11):1691-8).

In some embodiments, glycoconjugate targets of the present invention are glycoprotein and/or proteoglycans. Glycoproteins refer to any proteins that are covalently bonded with glycans. Proteoglycans are a class of proteins that are heavily glycosylated with glycans that often carry a negative charge. This property makes them very hydrophilic and important components of connective tissue.

### Recombinant Antibodies

Recombinant antibodies (e.g., glycan-interacting antibodies) of the invention may be generated using standard techniques known in the art. In some embodiments, recombinant antibodies may be anti-glycan antibodies. Further antibodies may be anti-STn antibodies (e.g. anti-GcSTn or anti-AcSTn antibodies). Recombinant antibodies of the invention may be produced using variable domains obtained from hybridoma cell-derived antibodies produced according to methods described herein. Heavy and light chain variable region cDNA sequences of antibodies may be determined using standard biochemical techniques. Total RNA may be extracted from antibody-producing hybridoma cells and converted to cDNA by reverse transcriptase (RT) polymerase chain reaction (PCR). PCR amplification may be carried out on resulting cDNA to amplify variable region genes. Such amplification may comprise the use of primers specific for amplification of heavy and light chain sequences. In other embodiments, recombinant antibodies may be produced using variable domains obtained from other sources. This includes the use of variable domains selected from one or more antibody fragment library, such as an scFv library used in antigen panning. Resulting PCR products may then be subcloned into plasmids for sequence analysis. Once sequenced, antibody coding sequences may be placed into expression vectors. For humanization, coding sequences for human heavy and light chain constant domains may be used to substitute for homologous murine sequences. The resulting constructs may then be transfected into mammalian cells for large scale translation.

### Anti-Tn Antibodies

In some embodiments, recombinant antibodies of the invention (e.g., glycan-interacting antibodies) may be anti-Tn antibodies. Such antibodies may bind to targets comprising Tn. Anti-Tn antibodies may be specific for Tn or may bind other modified forms of Tn, such as Tn linked to other moieties, including, but not limited to additional carbohydrate residues. In some cases anti-Tn antibodies may be anti-sialyl-Tn antibodies. Such antibodies may bind to targets comprising sialylated Tn comprising Neu5Ac and/or targets comprising sialylated Tn comprising Neu5Gc. Some anti-Tn antibodies may bind specifically to clusters of Tn antigen.

### Anti-STn Antibodies

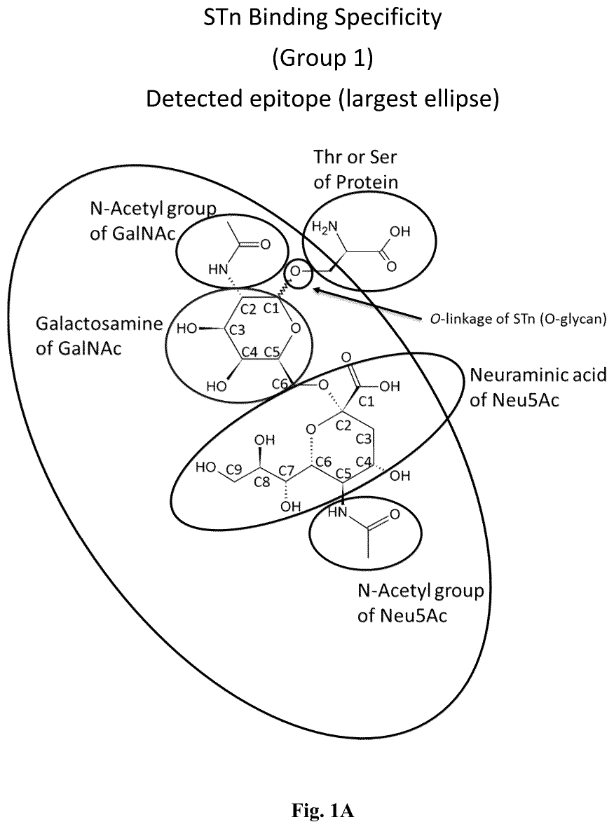

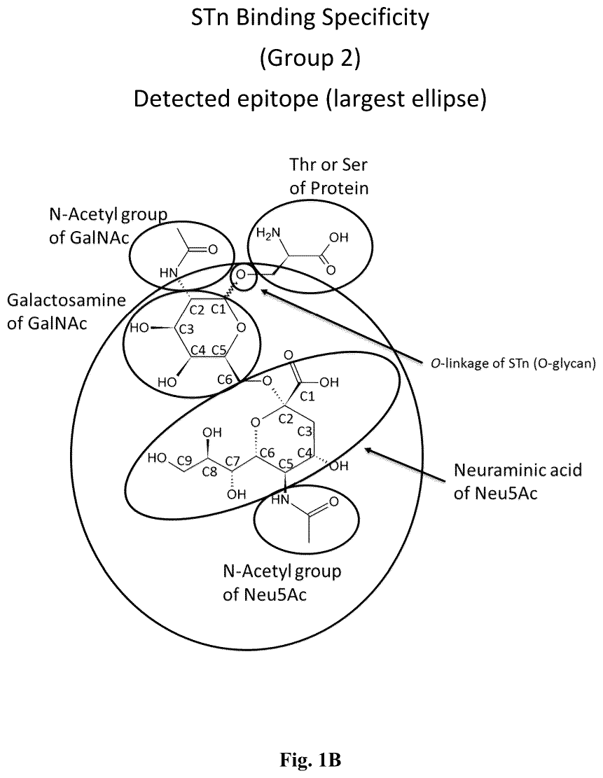

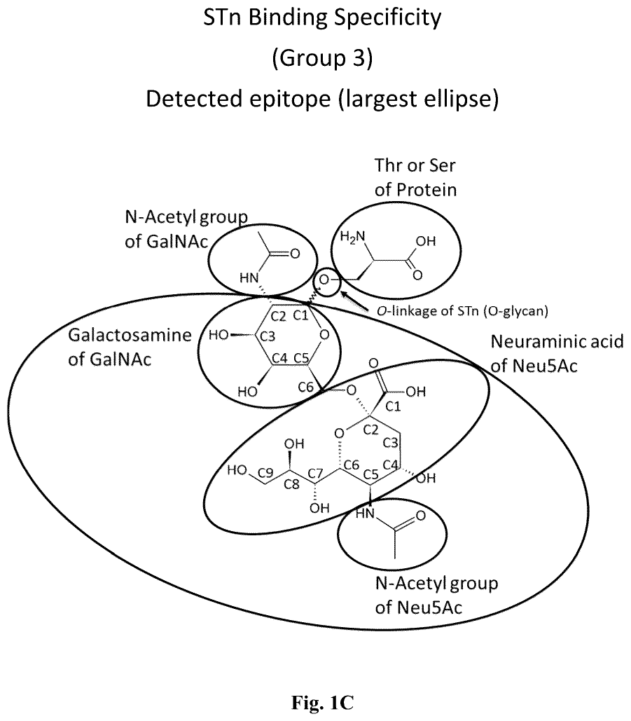

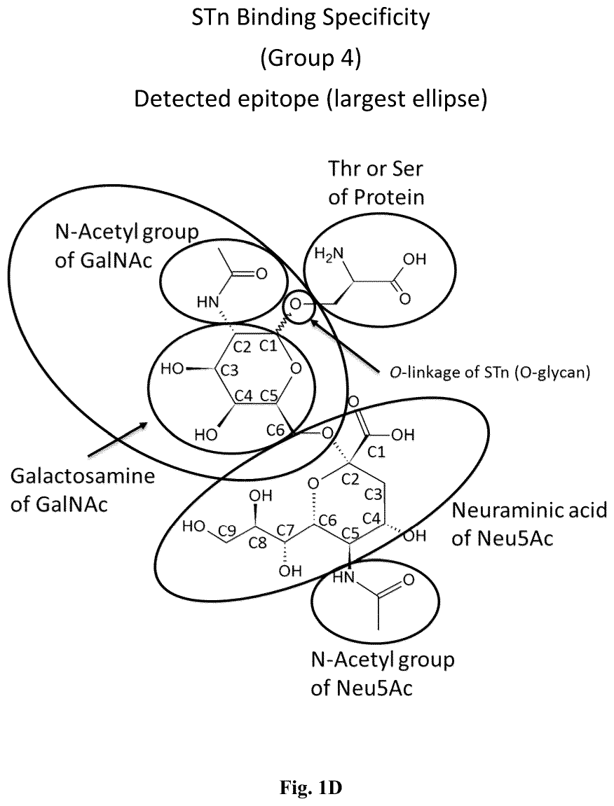

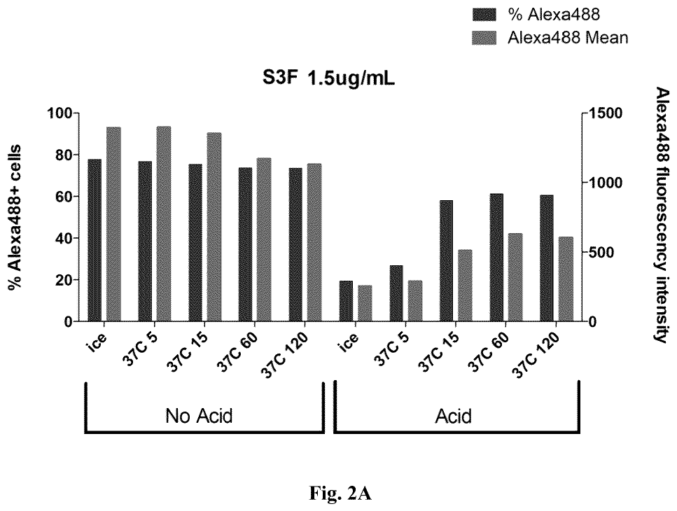

In some embodiments, antibodies of the invention (e.g., glycan-interacting antibodies) may specifically bind to antigens comprising STn. Anti-STn antibodies of the invention may be categorized by their binding to specific portions of STn antigens and/or by their specificity for AcSTn versus GcSTn. In some cases, anti-STn antibodies of the invention are Group 1 antibodies. “Group 1” antibodies according to the invention are antibodies capable of binding AcSTn and GcSTn. Such antibodies may also be referred to herein as pan-STn antibodies due to their ability to associate with a wider range of STn structures. In some embodiments, Group 1 antibodies may associate with the portion of STn indicated by the large oval in FIG. 1A. In some cases, anti-STn antibodies of the invention are Group 2 antibodies. “Group 2” antibodies, according to the invention, are antibodies capable of binding STn as well as some related structures that include an O-linkage to serine or threonine. In some embodiments, Group 2 antibodies may associate with glycans comprising a sialylated galactose residue. In some cases, Group 2 antibodies may associate with the portion of STn indicated by the large oval in FIG. 1B. Some Group 2 antibodies preferably bind to structures with AcSTn over structures with GcSTn. Further anti-STn antibodies may be Group 3 antibodies. As referred to herein, “Group 3” antibodies are antibodies capable of binding STn, but may also bind a broader set of related structures. Unlike Group 2 antibodies, Group 3 antibodies do not require that such structures have an O-linkage to serine or threonine. In some embodiments, Group 3 antibodies may associate with the portion of STn indicated by the large oval in FIG. 1C. Finally, some anti-STn antibodies of the invention may be Group 4 antibodies. As referred to herein, “Group 4” antibodies are capable of binding to both AcSTn and GcSTn as well as the un-sialylated Tn antigen, and therefore have broader specificity. In some embodiments, Group 4 antibodies may associate with the portion of STn indicated by the large oval in FIG. 1D.

In some cases, anti-STn antibodies of the invention may bind specifically to clusters of STn on a particular antigen or cell surface. Some such antibodies may recognize epitopes formed by the clustering of STn, including epitopes that include areas of contact between neighboring STn structures. Such epitopes may be formed by the clustering of 2, 3, 4, 5, 6, 7, 8, 9, 10 or more STn structures.

### Antibody Components

In some cases, antibodies or antigen binding fragments thereof of the invention may comprise variable domain and/or CDR amino acid sequences provided herein. Some antibodies or antigen binding fragments may comprise different combinations of such sequences. In some cases, antibodies or antigen binding fragments of the invention may comprise one or more of the variable domain sequences listed in Table 2. Residues indicated with an “X” may be absent or selected from any amino acid residues. In some cases, antibodies or antigen binding fragments thereof may comprise an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the variable domain sequences listed in Table 2. In some cases, antibodies or antigen binding fragments thereof of the invention may comprise an amino acid sequence comprising one or more fragments of any of the sequences listed in Table 2.

TABLE 2
Variable domain sequences

| Antibody | | | SEQ |
|---|---|---|---|
| ID | Variable | | ID |
| Number | chain | Sequence | NO |
| 18D2 | Heavy | QVTLKESGPGILQPSQTLSLTCSFSGFSLSTSNMGIGWIR | 1 |
| | chain | QPSGKGLEWLAHIWWHDDKYYNPSLKSRLTISKDISNN | |
| | | QVFLKITSVDTADTATYYCAQVPFYYGTSFDVWGTGT | |
| | | TVTVSS | |
| 18D2 | Light | DIVMTQSPATLSVTPGDRVSLSCRASQSISDYLHWYQQ | 2 |
| | chain 1 | KSHESPRLLIKYASQSISGIPSRFSGSGSGSDFTLSINSVEP | |
| | | EDVGVYYCQNGHSFPLTFGAGTKLELK | |
| 18D2 | Light | QIVLTQSPAIMSASPGETVTMTCSASSSITYMHWYQQK | 3 |
| | chain 2 | PGTSPKRWIYDTSKLASGVPARFSGSGSGTSYSLTISSM | |
| | | EAEDAATYYCHQRSSYTFGGGTKLEIKR | |
| 18C7 | Heavy | QVTLKESGPGILQPSQTLSLTCSFSGFSLSTFGMGVGWI | 4 |
| | chain | RQPSGKGLEWLAHIWWDDDKYYNPALKSRLTISKDTS | |
| | | KNQVFLKIANVDTADTATYYCARIAYYYGSERDYWGQ | |
| | | GTTLTVSS | |
| 18C7 | Light | QIVLTQSPAIMSASPGEKVTMTCSASSSISYMHWYHQK | 5 |
| | chain | PGTSPKRWIYDTSKLASGVPARFSGSGSGTSYSLTISSM | |
| | | EAEDAATYYCHQRSSYTFGGGTKLEIKR | |
| 10A5- | Heavy | QVQLKESGPGLVAPSQSLSITCTVSGFSLTSYGVSWVRQ | 6 |
| 2A12 | chain | PPGKGLEWLGVIWGDGSTNYHSSLISRLSISKDNSKSQV | |
| | | FLKLNSLQTDDTATYYCARAFVYWGQGTLVTVSA | |
| 10A5- | Light | QIVLTQSPAIMSASPGEKVTMTCSASSSVSYIHWYQQKS | 7 |
| 2A12 | chain | GTSPKRWIYDTSKLASGVPARFSGSGSGTSYSLTISSME | |
| | | AEDAATYYCQQWSSNPPMLTFGAGTKLELK | |
| 8C11- | Heavy | QVQLKESGPGLVAPSQSLSITCTVSGFSLTSYGVSWVRQ | 8 |
| 1D10 | chain | PPGKGLEWLGVIWGDGSTNYHSALISRLIISKDNSKSQV | |
| | | FLKLNSLQTDDTATYYCTKGFTYWGQGTLVTVSA | |
| 8C11- | Light | QIVLTQSPAIMSASPGEKVTMTCSASSSVSYMHWYQQK | 9 |
| 1D10 | chain | SGTSPKRWIFDTSKLASGVPARFSGSGSGTSYSLTISSME | |
| | | AEDAATYYCQQWSSNLLTFGAGTKLELK | |
| 2D4-1B4 | Heavy | QVQLQESGPGLVAPSQSLSITCTVSGFSLISYGVNWVRQ | 10 |
| | chain | PPGKGLEWLGVIWGDGSTNYQSALISRLIISKDNSKSQV | |
| | | FLKLNSLQTDDTATYYCTKGFAYWGQGTLVTVSA | |
| 2D4-1B4 | Light | QIVLTQSPAIMSASPGEKVTMTCSASSSVSYMHWFQQK | 11 |
| | chain | SGTSPKRWIYDTSKLASGVPARFSGSGSGTSYSLTISSM | |
| | | EAEDAATYYCQQWSSNLLTFGAGTKLELK | |
| 7G9-1A8 | Heavy | QVQLKESGPGLVAPSQNLSITCTVSGFSLTSYGVNWVR | 12 |
| | chain | QPPGKGLEWLGVIWGDGSTNYHSALISRLIISKENSKSQ | |
| | | VFLKLNSLQTNDTATYYCTKGFVYWGQGTLVTVSA | |
| 7G9-1A8 | Light | QIVLTQSPAIMSASPGEKVTMTCSASSSVSYMHWYQQK | 9 |
| | chain | SGTSPKRWIFDTSKLASGVPARFSGSGSGTSYSLTISSME | |
| | | AEDAATYYCQQWSSNLLTFGAGTKLELK | |
| 1A12-2B2 | Heavy | QVQLKESGPGLVAPSQSLSITCTVSGFSLTSYGVSWVRQ | 13 |
| | chain | PPGKGLEWLGVIWGDGSTNYHSALISRLSISKDNSKSQ | |
| | | VFLKLNSLQTDDTATYYCAKGGYFDYWGQGTTLTVSS | |
| 1A12-2B2 | Light | QIVLTQSPAVMSASPGEKVAITCSASSSVSYMHWFQQK | 14 |
| | chain | PGTSPKLWIYSTSNLASGVPARFSGSGSGTSYSLTISRME | |
| | | AEDAATYYCQQRSSYPWTFGGGTKLEIK | |

In some cases, antibodies or antigen binding fragments thereof of the invention may comprise one or more of the CDR amino acid sequences listed in Table 3. In some cases, antibodies or antigen binding fragments thereof may comprise an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the CDR sequences listed in Table 3. In some cases, antibodies or antigen binding fragments thereof of the invention may comprise an amino acid sequence comprising one or more fragments of any of the sequences listed in Table 3.

TABLE 3
CDR sequences

| | | | SEQ |
|---|---|---|---|
| Antibody ID | | | ID |
| Number | CDR | Sequence | NO |
| 18D2 | CDR-H1 | GFSLSTSNMG | 15 |
| 18C7 | CDR-H1 | GFSLSTFGMG | 16 |
| 10A5-2A12 | CDR-H1 | GFSLTSYG | 17 |
| 8C11-1D10 | CDR-H1 | GFSLTSYG | 17 |
| 2D4-1B4 | CDR-H1 | GFSLISYG | 18 |
| 7G9-1A8 | CDR-H1 | GFSLTSYG | 17 |
| 1A12-2B2 | CDR-H1 | GFSLTSYG | 17 |
| 18D2 | CDR-H2 | IWWHDDK | 19 |
| 18C7 | CDR-H2 | IWWDDDK | 20 |
| 10A5-2A12 | CDR-H2 | IWGDGST | 21 |
| 8C11-1D10 | CDR-H2 | IWGDGST | 21 |
| 2D4-1B4 | CDR-H2 | IWGDGST | 21 |
| 7G9-1A8 | CDR-H2 | IWGDGST | 21 |
| 1A12-2B2 | CDR-H2 | IWGDGST | 21 |
| 18D2 | CDR-H3 | AQVPFYYGTSFDV | 22 |
| 18C7 | CDR-H3 | ARIAYYYGSERDY | 23 |
| 10A5-2A12 | CDR-H3 | ARAFVY | 24 |
| 8C11-1D10 | CDR-H3 | TKGFTY | 25 |
| 2D4-1B4 | CDR-H3 | TKGFAY | 26 |
| 7G9-1A8 | CDR-H3 | TKGFVY | 27 |
| 1A12-2B2 | CDR-H3 | AKGGYFDY | 28 |
| 18C7 | CDR-L1 | SSISY | 29 |
| 10A5-2A12 | CDR-L1 | SSVSY | 30 |
| 8C11-1D10 | CDR-L1 | SSVSY | 30 |
| 2D4-1B4 | CDR-L1 | SSVSY | 30 |
| 7G9-1A8 | CDR-L1 | SSVSY | 30 |
| 1A12-2B2 | CDR-L1 | SSVSY | 30 |
| 18D2 | CDR-L1 | QSISDY | 31 |
| 18D2 | CDR-L1 | SSITY | 32 |
| 18C7 | CDR-L2 | DTS | 33 |
| 10A5-2A12 | CDR-L2 | DTS | 33 |
| 8C11-1D10 | CDR-L2 | DTS | 33 |
| 2D4-1B4 | CDR-L2 | DTS | 33 |
| 7G9-1A8 | CDR-L2 | DTS | 33 |
| 1A12-2B2 | CDR-L2 | STS | 34 |
| 18D2 | CDR-L2 | YAS | 35 |
| 18D2 | CDR-L2 | DTS | 33 |
| 18C7 | CDR-L3 | HQRSSYT | 36 |
| 10A5-2A12 | CDR-L3 | QQWSSNPPMLT | 37 |
| 8C11-1D10 | CDR-L3 | QQWSSNLLT | 38 |
| 2D4-1B4 | CDR-L3 | QQWSSNLLT | 38 |
| 7G9-1A8 | CDR-L3 | QQWSSNLLT | 38 |
| 1A12-2B2 | CDR-L3 | QQRSSYPWT | 39 |
| 18D2 | CDR-L3 | QNGHSFPLT | 40 |
| 18D2 | CDR-L3 | HQRSSYT | 36 |

In some cases, antibodies or antigen binding fragments of the invention may be encoded by a nucleotide sequence comprising one or more of the variable domain sequences listed in Table 4. In some cases, antibodies or antigen binding fragments thereof may be encoded by a nucleotide sequence comprising a sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the variable domain sequences listed in Table 4. In some cases, antibodies or antigen binding fragments thereof of the invention may be encoded by a nucleotide sequence comprising one or more fragments of any of the sequences listed in Table 4.

TABLE 4
Variable domain nucleotide sequences

| Antibody | | | SEQ |
|---|---|---|---|
| ID | Variable | | ID |
| Number | chain | Sequence | NO |
| 18D2 | Heavy | CAGGTTACTCTGAAAGAGTCTGGCCCTGGGATATTGCA | 41 |
| | chain | GCCCTCCCAGACCCTCAGTCTGACTTGTTCTTTCTCTGG | |
| | | GTTTTCACTGAGCACTTCTAATATGGGTATAGGCTGGA | |
| | | TTCGTCAGCCTTCAGGGAAGGGTCTAGAGTGGCTGGC | |
| | | ACACATTTGGTGGCATGATGATAAGTACTATAACCCAT | |
| | | CCCTGAAGAGCCGGCTCACAATCTCCAAGGATATCTCC | |
| | | AACAACCAGGTATTCCTCAAGATCACCAGTGTGGACA | |
| | | CTGCAGATACTGCCACGTACTACTGTGCTCAAGTCCCG | |
| | | TTTTACTACGGAACCTCGTTCGATGTCTGGGGCACAGG | |
| | | GACCACGGTCACCGTCTCCTCA | |
| 18D2 | Light | GACATTGTGATGACTCAGTCTCCAGCCACCCTGTCTGT | 42 |
| | chain 1 | GACTCCAGGAGATAGAGTCTCTCTTTCCTGCAGGGCCA | |
| | | GCCAGAGTATTAGCGACTACTTACACTGGTATCAACA | |
| | | AAAATCACATGAGTCTCCAAGGCTTCTCATCAAATATG | |
| | | CTTCCCAATCCATCTCTGGGATCCCCTCCAGGTTCAGT | |
| | | GGCAGTGGATCAGGGTCAGATTTCACTCTCAGTATCAA | |
| | | CAGTGTGGAACCTGAAGATGTTGGAGTGTATTACTGTC | |
| | | AAAATGGTCACAGCTTTCCTCTCACGTTCGGTGCTGGG | |
| | | ACCAAGCTGGAGCTGAAAC | |
| 18D2 | Light | CAAATTGTTCTCACCCAGTCTCCAGCAATCATGTCTGC | 43 |
| | chain 2 | ATCTCCAGGGGAGACGGTCACCATGACCTGCAGTGCC | |
| | | AGCTCAAGTATAACTTACATGCACTGGTACCAGCAGA | |
| | | AGCCAGGCACCTCCCCCAAAAGATGGATTTATGACAC | |
| | | ATCCAAACTGGCTTCTGGAGTCCCTGCTCGCTTCAGTG | |
| | | GCAGTGGGTCTGGGACCTCTTATTCTCTCACAATCAGC | |
| | | AGCATGGAGGCTGAAGATGCTGCCACTTATTACTGCC | |
| | | ATCAGCGGAGTAGTTACACGTTCGGAGGGGGGACCAA | |
| | | GCTGGAAATAAAACG | |
| 18C7 | Heavy | CAGGTTACTCTGAAAGAGTCTGGCCCTGGGATATTGCA | 44 |
| | chain | GCCCTCCCAGACCCTCAGTCTGACTTGTTCTTTCTCTGG | |
| | | GTTTTCACTGAGCACTTTTGGTATGGGTGTAGGCTGGA | |
| | | TTCGTCAGCCTTCAGGGAAGGGTCTGGAGTGGCTGGC | |
| | | ACACATTTGGTGGGATGATGATAAGTACTATAACCCA | |
| | | GCCCTGAAGAGTCGGCTCACAATCTCCAAGGATACCT | |
| | | CCAAAAACCAGGTATTCCTCAAGATCGCCAATGTGGA | |
| | | CACTGCAGATACTGCCACATACTACTGTGCTCGAATAG | |
| | | CCTATTACTACGGTAGCGAGAGGGACTACTGGGGCCA | |
| | | AGGCACCACTCTCACAGTCTCCTCA | |
| 18C7 | Light | CAAATTGTTCTCACCCAGTCTCCAGCAATCATGTCTGC | 45 |
| | chain | ATCTCCAGGGGAGAAGGTCACCATGACCTGCAGTGCC | |
| | | AGCTCAAGTATAAGTTACATGCACTGGTACCACCAGA | |
| | | AGCCAGGCACCTCCCCCAAAAGATGGATTTATGACAC | |
| | | ATCCAAACTGGCTTCTGGAGTCCCTGCTCGCTTCAGTG | |
| | | GCAGTGGGTCTGGGACCTCTTATTCTCTCACAATCAGC | |
| | | AGCATGGAGGCTGAAGATGCTGCCACTTATTACTGCC | |
| | | ATCAGCGGAGTAGTTACACGTTCGGAGGGGGGACCAA | |
| | | GCTGGAAATAAAACG | |
| 18D2(2) | Heavy | ATGGACAGGCTTACTTCCTCATTCTTGCTACTGATTGT | 46 |
| | chain | CCCTGCATATGTCCTGTCCCAGGTTACTCTGAAAGAGT | |
| | | CTGGCCCTGGGATATTGCAGCCCTCCCAGACCCTCAGT | |
| | | CTGACTTGTTCTTTCTCTGGGTTTTCACTGAGCACTTCT | |
| | | AATATGGGTATAGGCTGGATTCGTCAGCCTTCAGGGA | |
| | | AGGGTCTAGAGTGGCTGGCACACATTTGGTGGCATGA | |
| | | TGATAAGTACTATAACCCATCCCTGAAGAGCCGGCTC | |
| | | ACAATCTCCAAGGATATCTCCAACAACCAGGTATTCCT | |
| | | CAAGATCACCAGTGTGGACACTGCAGATACTGCCACG | |
| | | TACTACTGTGCTCAAGTCCCGTTTTACTACGGAACCTC | |
| | | GTTCGATGTCTGGGGCACAGGGACCACGGTCACCGTC | |
| | | TCCTCAGCCAAAACGACACCCCCATCTGTCTATCCGCT | |
| | | CGCCCCTGGATCTGCTGCCCAAACTAACTCCATGGTGA | |
| | | CCCTGGGATGCCTGGTCAAGGGCTATTTCCCTGAGCCA | |
| | | GTGACAGTGACCTGGAACTCTGGATCCCTGTCCAGCG | |
| | | GTGTGCACACCTTCCCAGCTGTCCTGCAGTCTGACCTC | |
| | | TACACTCTGAGCAGCTCAGTGACTGTCCCCTCCAGCAC | |
| | | CTGGCCCAGCGAGACCGTCACCTGCAACGTTGCCCAC | |
| | | CCGGCCAGCAGCACCAAGGTGGACAAGACAATTGTGC | |
| | | CCAGGGATTGTGGTTGTAAGCCTTGCATATGTACAGTC | |
| | | CCAGAAGTATCATCTGTCTTCATCTTCCCCCCAAAGCC | |
| | | CAAGGATGTGCTCACCATTACTCTGACTCCTAAGGTCA | |
| | | CGTGTGTTGTGGTAGACATCAGCCAGGATGATCCCGA | |
| | | GGTCAGTTCAGCTGTTTGTAGATGATGTGGAAGTGCAC | |
| | | ACAGCTCAAAACAACCCCCCGAGAGGACATTTCACAA | |
| | | CATTTCCGCTCATCAGTGAATTTCCCATCTGCACAAGA | |
| | | CTGCTTAATGGCAAGAGTTAAATGCAGGTCAAAGGGC | |
| | | AGTTTCCTGCCCCATCAAAAACTTTTCAAAA | |
| 18D2(2) | Light | ATGGATTTTCAAGTGCAGATTTTCAGCTTCCTGCTAAT | 47 |
| | chain | CAGTGCCTCAGTCATACTGTCCAGAGGACAAATTGTTC | |
| | | TCACCCAGTCTCCAGCAATCATGTCTGCATCTCCAGGG | |
| | | GAGACGGTCACCATGACCTGCAGTGCCAGCTCAAGTA | |
| | | TAACTTACATGCACTGGTACCAGCAGAAGCCAGGCAC | |
| | | CTCCCCCAAAAGATGGATTTATGACACATCCAAACTG | |
| | | GCTTCTGGAGTCCCTGCTCGCTTCAGTGGCAGTGGGTC | |
| | | TGGGACCTCTTATTCTCTCACAATCAGCAGCATGGAGG | |
| | | CTGAAGATGCTGCCACTTATTACTGCCATCAGCGGAGT | |
| | | AGTTACACGTTCGGAGGGGGGACCAAGCTGGAAATAA | |
| | | AACGGGCTGATGCTGCACCAACTGTATCCATCTTCCCA | |
| | | CCATCCAGTGAGCAGTTAACATCTGGAGGTGCCTCAGT | |
| | | CGTGTGCTTCTTGAACAACTTCTACCCCAAAGACATCA | |
| | | ATGTCAAGTGGAAGATTGATGGCAGTGAACGACAAAA | |
| | | TGGCGTCCTGAACAGTTGGACTGATCAGGACAGCAAA | |
| | | GACAGCACCTACAGCATGAGCAGCACCCTCACGTTGA | |
| | | CCAAGGACGAGTATGAACGACATAACAGCTATACCTG | |
| | | TGAGGCCACTCACAAGACATCAACTTCACCCATTGTCA | |
| | | AGAGCTTCAACAGGAATGAGTGTTAG | |
| 18C7(2) | Heavy | ATGGACAGGCTTACTTCCTCATTCCTGTTACTGATTGT | 48 |
| | chain | CCCTGCATATGTCCTGTCCCAGGTTACTCTGAAAGAGT | |
| | | CTGGCCCTGGGATATTGCAGCCCTCCCAGACCCTCAGT | |
| | | CTGACTTGTTCTTTCTCTGGGTTTTCACTGAGCACTTTT | |
| | | GGTATGGGTGTAGGCTGGATTCGTCAGCCTTCAGGGA | |
| | | AGGGTCTGGAGTGGCTGGCACACATTTGGTGGGATGA | |
| | | TGATAAGTACTATAACCCAGCCCTGAAGAGTCGGCTC | |
| | | ACAATCTCCAAGGATACCTCCAAAAACCAGGTATTCCT | |
| | | CAAGATCGCCAATGTGGACACTGCAGATACTGCCACA | |
| | | TACTACTGTGCTCGAATAGCCTATTACTACGGTAGCGA | |
| | | GAGGGACTACTGGGGCCAAGGCACCACTCTCACAGTC | |
| | | TCCTCAGCCAAAACGACACCCCCATCTGTCTATCCGCT | |
| | | CGCCCCTGGATCTGCTGCCCAAACTAACTCCATGGTGA | |
| | | CCCTGGGATGCCTGGTCAAGGGCTATTTCCCTGAGCCA | |
| | | GTGACAGTGACCTGGAACTCTGGATCCCTGTCCAGCG | |
| | | GTGTGCACACCTTCCCAGCTGTCCTGCAGTCTGACCTC | |
| | | TACACTCTGAGCAGCTCAGTGACTGTCCCCTCCAGCAC | |
| | | CTGGCCCAGCGAGACCGTCACCTGCAACGTTGCCCAC | |
| | | CCGGCCAGCAGCACCAAGGTGGACAAGAAAATTGTGC | |
| | | CCAGGGATTGTGGTTGTAAGCCTTGCATATGTACAGTC | |
| | | CCAGAAGTATCATCTGTCTTCATCTTCCCCCCAAAGCC | |
| | | CAAGGATGTGCTCACCATTACTCTGACTCCTAAGGTCA | |
| | | CGTGTGTTGTGGTAGACATCAGCAAGGATGATCCCGA | |
| | | GGTCCAGTTCAGCTGGTTTGTAGATGATGTGGAAGTGC | |
| | | ACACAGCTCAGACGNCACCCCGGGGAGAGCAGTTTCA | |
| | | CAGCACTTTCCGCTCAGTCAGTGAACTTCCCATCATGC | |
| | | ACCANGACTGGGCTCATGGNCAGGAGTTCAANTGCAG | |
| | | GTCACAGTGCAGCTTTCCTGCCCCATCGAGAAACATCT | |
| | | CCNAAACAAGGCGACGAAAGCTCACAGGGTACACATT | |
| | | CCACTCCCNAGAGCAATGCCAGATAAGTCATCTGACT | |
| | | GCTGATACAACTCTTCTGAAAATACTGTGAATGCATGG | |
| | | ATGCCACCACGAAAATCAAACCTCGCCCTTGGACNAT | |
| | | GGCTTATTTTACCAGCTAGTCAAAACCTGGGGGGAATT | |
| | | TCCCGTCTGTT | |
| 18C7(2) | Light | ATGGTTTTCACACCTCAGATACTTGGACTTATGCTTTTT | 49 |
| | chain | TGGATTTCAGCCTCCAGATGTGACATTGTGATGACTCA | |
| | | GTCTCCAGCCACCCTGTCTGTGACTCCAGGAGATAGAG | |
| | | TCTCTCTTTCCTGCAGGGCCAGCCAGAGTATTAGCGAC | |
| | | TACTTACACTGGTATCAACAAAAATCACATGAGTCTCC | |
| | | AAGGCTTCTCATCAAATATGCTTCCCAATCCATCTCTG | |
| | | GGATCCCCTCCAGGTTCAGTGGCAGTGGATCAGGGTC | |
| | | AGATTTCACTCTCAGTATCAACAGTGTGGAACCTGAAG | |
| | | ATGTTGGAGTGTATTACTGTCAAAATGGTCACAGCTTT | |
| | | CCTCTCACGTTCGGTGCTGGGACCAAGCTGGAAATAA | |
| | | AACGGGCTGATGCTGCACCAACTGTATCCATCTTCCCA | |
| | | CCATCCAGTGAGCAGTTAACATCTGGAGGTGCCTCAGT | |
| | | CGTGTGCTTCTTGAACAACTTCTACCCCAAAGACATCA | |
| | | ATGTCAAGTGGAAGATTGATGGCAGTGAACGACAAAA | |
| | | TGGCGTCCTGAACAGTTGGACTGATCAGGACAGCAAA | |
| | | GACAGCACCTACAGCATGAGCAGCACCCTCACGTTGA | |
| | | CCAAGGACGAGTATGAACGACATAACAGCTATACCTG | |
| | | TGAGGCCACTCACAAGACATCAACTTCACCCATTGTCA | |
| | | AGAGCTTCAACAGGAATGAGTGTTAG | |
| 10A5- | Heavy | CAGGTGCAGCTGAAGGAGTCAGGACCTGGCCTGGTGG | 50 |
| 2A12 | chain | CGCCCTCACAGAGCCTGTCCATCACATGCACTGTCTCA | |
| | | GGGTTCTCATTAACCAGCTATGGTGTAAGCTGGGTTCG | |
| | | CCAGCCTCCAGGAAAGGGTCTGGAGTGGCTGGGAGTA | |
| | | ATATGGGGTGACGGAAGCACAAATTATCATTCATCTCT | |
| | | CATATCCAGACTGAGCATCAGCAAGGATAACTCCAAG | |
| | | AGCCAAGTTTTCTTAAAACTGAACAGTCTGCAAACTGA | |
| | | TGACACAGCCACGTACTACTGTGCCAGAGCCTTTGTTT | |
| | | ACTGGGGCCAAGGGACTCTGGTCACTGTCTCTGCA | |
| 10A5- | Light | CAAATTGTTCTCACCCAGTCTCCAGCAATCATGTCTGC | 51 |
| 2A12 | chain | ATCTCCAGGGGAGAAGGTCACCATGACCTGCAGTGCC | |
| | | AGCTCAAGTGTAAGTTACATACACTGGTACCAGCAGA | |
| | | AGTCAGGCACCTCCCCCAAAAGATGGATTTATGACAC | |
| | | ATCCAAACTGGCTTCTGGAGTCCCTGCTCGCTTCAGTG | |
| | | GCAGTGGGTCTGGGACCTCTTACTCTCTCACAATCAGC | |
| | | AGCATGGAGGCTGAAGATGCTGCCACTTATTACTGCC | |
| | | AGCAGTGGAGTAGTAACCCACCCATGCTCACGTTCGG | |
| | | TGCTGGGACCAAGCTGGAGCTGAAAC | |
| 8C11- | Heavy | CAGGTGCAGCTGAAGGAGTCAGGACCTGGCCTGGTGG | 52 |
| 1D10 | chain | CGCCCTCACAGAGCCTGTCCATCACATGCACTGTCTCA | |
| | | GGGTTCTCATTAACCAGCTATGGTGTAAGCTGGGTTCG | |
| | | CCAGCCTCCAGGAAAGGGTCTGGAGTGGCTGGGAGTA | |
| | | ATATGGGGTGACGGGAGCACAAATTATCATTCAGCTC | |
| | | TCATATCCAGACTGATCATCAGCAAGGATAACTCCAA | |
| | | GAGCCAAGTTTTCTTAAAACTGAACAGTCTGCAAACTG | |
| | | ATGACACAGCCACCTACTACTGTACCAAAGGCTTTACT | |
| | | TACTGGGGCCAGGGGACTCTGGTCACTGTCTCTGCA | |
| 8C11- | Light | CAAATTGTTCTCACCCAGTCTCCAGCAATCATGTCTGC | 53 |
| 1D10 | chain | ATCTCCAGGGGAGAAGGTCACCATGACCTGCAGTGCC | |
| | | AGCTCAAGTGTAAGTTACATGCACTGGTACCAGCAGA | |
| | | AGTCAGGCACCTCCCCCAAAAGATGGATTTTTGACAC | |
| | | ATCCAAACTGGCTTCTGGAGTCCCTGCTCGCTTCAGTG | |
| | | GCAGTGGGTCTGGGACCTCTTACTCTCTCACAATCAGC | |
| | | AGCATGGAGGCTGAAGATGCTGCCACTTATTACTGCC | |
| | | AGCAGTGGAGTAGTAACCTGCTCACGTTCGGTGCTGG | |
| | | GACCAAGCTGGAGCTGAAAC | |
| 2D4-1B4 | Heavy | CAGGTGCAGCTGCAGGAGTCAGGACCTGGCCTGGTGG | 54 |
| | chain | CGCCCTCACAGAGCCTGTCCATCACATGCACTGTCTCA | |
| | | GGGTTCTCATTAATCAGCTATGGTGTAAACTGGGTTCG | |
| | | CCAGCCTCCAGGAAAGGGTCTGGAGTGGCTGGGAGTG | |
| | | ATATGGGGTGACGGGAGCACAAATTATCAGTCAGCTC | |
| | | TCATATCCAGACTGATCATCAGCAAGGATAACTCCAA | |
| | | GAGCCAAGTTTTCTTAAAACTGAACAGTCTGCAAACTG | |
| | | ATGACACAGCCACGTACTACTGTACCAAAGGCTTTGCT | |
| | | TACTGGGGCCAAGGGACTCTGGTCACTGTCTCTGCA | |
| 2D4-1B4 | Light | CAAATTGTTCTCACCCAGTCTCCAGCAATCATGTCTGC | 55 |
| | chain | ATCTCCAGGGGAGAAGGTCACCATGACCTGCAGTGCC | |
| | | AGCTCAAGTGTAAGTTACATGCACTGGTTCCAGCAGA | |
| | | AGTCAGGCACCTCCCCCAAAAGATGGATTTATGACAC | |
| | | ATCCAAACTGGCTTCTGGAGTCCCTGCTCGCTTCAGTG | |
| | | GCAGTGGGTCTGGGACCTCTTACTCTCTCACAATCAGC | |
| | | AGCATGGAGGCTGAAGATGCTGCCACTTATTACTGCC | |
| | | AGCAGTGGAGTAGTAACCTGCTCACGTTCGGTGCTGG | |
| | | GACCAAGCTGGAGCTGAAAC | |
| 7G9-1A8 | Heavy | CAGGTGCAGCTGAAGGAGTCAGGACCTGGCCTGGTGG | 56 |
| | chain | CGCCCTCACAGAACCTGTCCATCACATGCACTGTCTCA | |
| | | GGGTTCTCATTAACCAGTTATGGTGTAAACTGGGTTCG | |
| | | CCAGCCTCCAGGAAAGGGTCTGGAGTGGCTGGGAGTA | |
| | | ATATGGGGTGACGGGAGCACAAATTATCATTCAGCTC | |
| | | TCATTTCCAGACTGATCATCAGCAAGGAAAACTCCAA | |
| | | GAGCCAAGTTTTCTTAAAACTGAACAGTCTGCAAACTA | |
| | | ATGACACAGCCACGTATTACTGTACCAAAGGCTTTGTT | |
| | | TACTGGGGCCAAGGGACTCTGGTCACTGTCTCTGCA | |
| 7G9-1A8 | Light | CAAATTGTTCTCACCCAGTCTCCAGCAATCATGTCTGC | 53 |
| | chain | ATCTCCAGGGGAGAAGGTCACCATGACCTGCAGTGCC | |
| | | AGCTCAAGTGTAAGTTACATGCACTGGTACCAGCAGA | |
| | | AGTCAGGCACCTCCCCCAAAAGATGGATTTTTGACAC | |
| | | ATCCAAACTGGCTTCTGGAGTCCCTGCTCGCTTCAGTG | |
| | | GCAGTGGGTCTGGGACCTCTTACTCTCTCACAATCAGC | |
| | | AGCATGGAGGCTGAAGATGCTGCCACTTATTACTGCC | |
| | | AGCAGTGGAGTAGTAACCTGCTCACGTTCGGTGCTGG | |
| | | GACCAAGCTGGAGCTGAAAC | |
| 1A12-2B2 | Heavy | CAGGTGCAGCTGAAGGAGTCAGGACCTGGCCTGGTGG | 57 |
| | chain | CGCCCTCACAGAGCCTGTCCATCACATGCACTGTCTCA | |
| | | GGGTTCTCATTAACCAGCTATGGTGTAAGCTGGGTTCG | |
| | | CCAGCCTCCAGGAAAGGGTCTGGAGTGGCTGGGAGTA | |
| | | ATATGGGGTGACGGGAGCACAAATTATCATTCAGCTC | |
| | | TCATATCCAGACTGAGCATCAGCAAGGATAACTCCAA | |
| | | GAGCCAAGTTTTCTTAAAACTGAACAGTCTGCAAACTG | |
| | | ATGACACAGCCACGTACTACTGTGCCAAAGGGGGCTA | |
| | | CTTTGACTACTGGGGCCAAGGCACCACTCTCACAGTCT | |
| | | CCTCA | |
| 1A12-2B2 | Light | CAAATTGTTCTCACCCAGTCTCCAGCAGTCATGTCTGC | 58 |
| | chain | ATCTCCAGGGGAGAAGGTCGCCATAACCTGCAGTGCC | |
| | | AGCTCAAGTGTAAGTTACATGCACTGGTTCCAGCAGA | |
| | | AGCCAGGCACTTCTCCCAAACTCTGGATTTATAGCACA | |
| | | TCCAACCTGGCTTCTGGAGTCCCTGCTCGCTTCAGTGG | |
| | | CAGTGGATCTGGGACCTCTTACTCTCTCACAATCAGCC | |
| | | GAATGGAGGCTGAAGATGCTGCCACTTATTACTGCCA | |
| | | GCAAAGGAGTAGTTACCCGTGGACGTTCGGTGGAGGC | |
| | | ACCAAGCTGGAAATCAAAC | |

In some cases, antibodies or antigen binding fragments of the invention may comprise one or more of the variable domain sequences listed in Table 5. In some cases, antibodies or antigen binding fragments thereof may comprise an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the variable domain sequences listed in Table 5. In some cases, antibodies or antigen binding fragments thereof of the invention may comprise an amino acid sequence comprising one or more fragments of any of the sequences listed in Table 5.

TABLE 5
Variable domain sequences

| Antibody | | | SEQ |
|---|---|---|---|
| ID | Variable | | ID |
| Number | chain | Sequence | NO |
| 3F1 | Heavy | QVQLQQSDAELVKPGASVKISCKASGYTFTDHAI | 59 |
| | chain | HWVKQKPEQGLDWIGYISPGNGDIKYNEKFKDK | |
| | | VTLTADKSSSTACMHLNSLTSEDSAVYFCKRSLL | |
| | | ALDYWGQGTTLTVSS | |
| 3F1 | Light | DIVMTQSHKFMSTSVGDRVSITCKASQDVGTNIA | 60 |
| | chain 1 | WYQQKPGRSPKVLIYSASTRHTGVPDRFTGSGSG | |
| | | TDFTLTISNVQSEDLTDYFCQQYSSFPLTFGVGTK | |
| | | LELK | |

In some cases, antibodies or antigen binding fragments of the invention may comprise the IgG2a heavy chain and/or kappa light chain constant domain sequences listed in Table 6. In some cases, antibodies or fragments thereof may comprise an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the constant domain sequences listed in Table 6. In some cases, antibodies or fragments thereof of the invention may comprise an amino acid sequence comprising one or more fragments of any of the sequences listed in Table 6.

TABLE 6
Constant domain sequences

| | | SEQ |
|---|---|---|
| | | ID |
| Domain | Sequence | NO |
| IgG2a | AKTTAPSVYPLAPVCGDTTGSSVTLGCLVKGYFPEPV | 61 |
| heavy | TLTWNSGSLSSGVHTFPAVLQSDLYTLSSSVTVTSST | |
| chain | WPSQSITCNVAHPASSTKVDKKIEPRGPTIKPCPPCKC | |
| constant | PAPNLLGGPSVFIFPPKIKDVLMISLSPIVTCVVVDVSE | |
| domain | DDPDVQISWFVNNVEVHTAQTQTHREDYNSTLRVVS | |
| | ALPIQHQDWMSGKEFKCKVNNKDLPAPIERTISKPKG | |
| | SVRAPQVYVLPPPEEEMTKKQVTLTCMVTDFMPEDI | |
| | YVEWTNNGKTELNYKNTEPVLDSDGSYFMYSKLRV | |
| | EKKNWVERNSYSCSVVHEGLHNHHTTKSFSRTPGK | |
| kappa | RADAAPTVSIFPPSSEQLTSGGASVVCFLNNFYPKDIN | 62 |
| light | VKWKIDGSERQNGVLNSWTDQDSKDSTYSMSSTLTL | |
| chain | TKDEYERHNSYTCEATHKTSTSPIVKSFNRNEC | |
| constant | | |
| domain | | |

In some cases, antibodies may comprise the heavy chain and/or light chain amino acid sequences listed in Table 7 or be encoded by one or more of the heavy and/or light chain nucleotide sequences listed in Table 7. In some cases, antibodies or fragments thereof may comprise an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.90%, from about 95% to about 99.9%, about 97%, about 97.5%, about 98%, about 98.5%, about 99%, about 99.5%, about 99.6%, about 99.7% or about 99.8%) with one or more of the amino acid sequences listed in Table 7. In some cases, antibodies or fragments thereof of the invention may comprise an amino acid sequence comprising one or more fragments ofany of the sequences listed in Table 7. In some cases, antibodies or fragments thereof may be encoded by a nucleotide sequence with from about 5000 to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.90%, from about 95% to about 99.9%, about 97%, about 97.5%, about 98%, about 98.5%, about 99%, about 99.5%, about 99.6%, about 99.7% or about 99.8%) with one or more of the nucleotide sequences listed in Table 7.

TABLE 7
Constant domain sequences

| | | SEQ |
|---|---|---|
| | | ID |
| Domain | Sequence | NO |
| S3F full | QVQLQQSDAELVKPGASVKISCKASGYTFTDHAIHWVKQKP | 63 |
| length | EQGLDWIGYISPGNGDIKYNEKFKDKVTLTADKSSSTACMH | |
| heavy | LNSLTSEDSAVYFCKRSLLALDYWGQGTTLTVSSAKTTAPSV | |
| chain | YPLAPVCGDTTGSSVTLGCLVKGYFPEPVTLTWNSGSLSSGV | |
| | HTFPAVLQSDLYTLSSSVTVTSSTWPSQSITCNVAHPASSTKV | |
| | DKKIEPRGPTIKPCPPCKCPAPNLLGGPSVFIFPPKIKDVLMISL | |
| | SPIVTCVVVDVSEDDPDVQISWFVNNVEVHTAQTQTHREDY | |
| | NSTLRVVSALPIQHQDWMSGKEFKCKVNNKDLPAPIERTISK | |
| | PKGSVRAPQVYVLPPPEEEMTKKQVTLTCMVTDFMPEDIYV | |
| | EWTNNGKTELNYKNTEPVLDSDGSYFMYSKLRVEKKNWVE | |
| | RNSYSCSVVHEGLHNHHTTKSFSRTPGK | |
| S3F full | DIVMTQSHKFMSTSVGDRVSITCKASQDVGTNIAWYQQKPG | 64 |
| length | RSPKVLIYSASTRHTGVPDRFTGSGSGTDFTLTISNVQSEDLT | |
| light chain | DYFCQQYSSFPLTFGVGTKLELKRADAAPTVSIFPPSSEQLTS | |
| | GGASVVCFLNNFYPKDINVKWKIDGSERQNGVLNSWTDQDS | |
| | KDSTYSMSSTLTLTKDEYERHNSYTCEATHKTSTSPIVKSFNR | |
| | NEC | |
| S3F full | ATGGAGACCGACACCCTGCTGCTCTGGGTGCTGCTGCTCT | 65 |
| length | GGGTGCCCGGCTCCACCGGACAGGTTCAGCTGCAGCAGTC | |
| heavy | TGACGCTGAGTTGGTGAAACCTGGGGCTTCAGTGAAGATA | |
| chain | TCCTGCAAGGCTTCTGGCTACACCTTCACTGACCATGCTAT | |
| nucleotide | TCACTGGGTGAAGCAAAAGCCTGAACAGGGCCTGGACTG | |
| sequence | GATTGGATATATTTCTCCCGGAAATGGTGATATTAAGTAC | |
| | AATGAGAAGTTCAAGGACAAGGTCACACTGACTGCAGAC | |
| | AAATCCTCCAGCACTGCCTGCATGCACCTCAACAGCCTGA | |
| | CATCTGAGGATTCTGCAGTGTATTTCTGCAAAAGATCCCT | |
| | ACTAGCTCTTGACTACTGGGGCCAAGGCACCACTCTCACA | |
| | GTCTCCTCAGCTAAAACAACAGCCCCATCGGTCTATCCAC | |
| | TGGCCCCTGTGTGTGGAGATACAACTGGCTCCTCGGTGAC | |
| | TCTAGGATGCCTGGTCAAGGGTTATTTCCCTGAGCCAGTG | |
| | ACCTTGACCTGGAACTCTGGTTCCCTGTCCAGTGGTGTGC | |
| | ACACCTTCCCAGCTGTCCTGCAGTCTGACCTCTACACCCTC | |
| | AGCTCAAGCGTGACTGTAACCAGCTCGACCTGGCCCAGCC | |
| | AGTCCATCACCTGCAATGTGGCCCACCCGGCAAGCAGCAC | |
| | CAAGGTGGACAAGAAAATTGAGCCCAGAGGGCCCACAAT | |
| | CAAGCCCTGTCCTCCATGCAAATGCCCAGCACCTAACCTC | |
| | TTGGGTGGACCATCCGTCTTCATCTTCCCTCCAAAGATCAA | |
| | GGATGTACTCATGATCTCCCTGAGCCCCATAGTCACATGT | |
| | GTAGTCGTTGATGTGAGCGAGGATGACCCAGATGTCCAGA | |
| | TCAGCTGGTTTGTGAACAACGTGGAAGTGCACACTGCTCA | |
| | GACACAGACGCATAGAGAGGATTACAACAGTACTCTCCG | |
| | GGTTGTCAGTGCCCTCCCCATCCAGCACCAGGACTGGATG | |
| | AGTGGCAAGGAGTTCAAATGCAAGGTCAACAACAAAGAC | |
| | CTCCCAGCGCCCATCGAGAGAACCATCTCAAAACCCAAAG | |
| | GGTCAGTAAGAGCTCCACAGGTATATGTCTTGCCTCCACC | |
| | AGAAGAGGAGATGACTAAGAAACAGGTCACTCTGACCTG | |
| | CATGGTCACAGACTTCATGCCTGAAGACATTTACGTGGAG | |
| | TGGACCAACAACGGGAAAACAGAGCTAAACTACAAGAAC | |
| | ACTGAACCAGTCCTGGACTCTGATGGTTCTTACTTCATGTA | |
| | CAGCAAGCTGAGAGTGGAGAAGAAGAACTGGGTGGAGAG | |
| | AAATAGCTACTCCTGTTCAGTGGTCCACGAGGGTCTGCAC | |
| | AATCACCACACGACTAAGAGCTTCTCCCGGACTCCGGGTA | |
| | AATAG | |
| S3F full | ATGGAGACCGACACCCTGCTGCTCTGGGTGCTGCTGCTCT | 66 |
| length | GGGTGCCCGGCTCCACCGGAGACATTGTGATGACCCAGTC | |
| light chain | TCACAAATTCATGTCCACATCAGTAGGAGACAGGGTCAGC | |
| nucleotide | ATCACCTGCAAGGCCAGTCAGGATGTGGGCACTAATATAG | |
| sequence | CCTGGTATCAACAGAAACCAGGCCGATCTCCTAAAGTACT | |
| | GATTTACTCGGCATCCACCCGGCACACTGGAGTCCCTGAT | |
| | CGCTTCACAGGCAGTGGATCTGGGACAGATTTCACTCTCA | |
| | CCATTAGCAATGTGCAGTCTGAAGACTTGACAGATTATTT | |
| | CTGTCAGCAATATAGCAGCTTTCCTCTCACGTTCGGTGTTG | |
| | GGACCAAGCTGGAGCTGAAACGGGCAGATGCTGCACCAA | |
| | CTGTATCCATCTTCCCACCATCCAGTGAGCAGTTAACATCT | |
| | GGAGGTGCCTCAGTCGTGTGCTTCTTGAACAACTTCTACC | |
| | CCAAAGACATCAATGTCAAGTGGAAGATTGATGGCAGTG | |
| | AACGACAAAATGGCGTCCTGAACAGTTGGACTGATCAGG | |
| | ACAGCAAAGACAGCACCTACAGCATGAGCAGCACCCTCA | |
| | CGTTGACCAAGGACGAGTATGAACGACATAACAGCTATA | |
| | CCTGTGAGGCCACTCACAAGACATCAACTTCACCCATTGT | |
| | CAAGAGCTTCAACAGGAATGAGTGTTGA | |

### IgG Synthesis

IgG antibodies (e.g. IgG1, IgG2, IgG3 or IgG4) comprising one or more variable domain and/or CDR amino acid sequences presented herein (or fragment or variants thereof) may be synthesized for further testing and/or product development. Such antibodies may be produced by insertion of one or more segments of cDNA encoding desired amino acid sequences into expression vectors suited for IgG production. Expression vectors may comprise mammalian expression vectors suitable for IgG expression in mammalian cells. Mammalian expression of IgGs may be carried out to ensure that antibodies produced comprise modifications (e.g. glycosylation) characteristic of mammalian proteins and/or to ensure that antibody preparations lack endotoxin and/or other contaminants that may be present in protein preparations from bacterial expression systems.

### Cancer-Related Targets

In some embodiments, targets of the present invention are cancer-related antigens or epitopes. As used herein, the term “cancer-related” is used to describe entities that may be in some way associated with cancer, cancerous cells and/or cancerous tissues. Many cancer-related antigens or epitopes comprising glycans have been identified that are expressed in correlation with tumor cells (Heimburg-Molinaro, J. et al., Cancer vaccines and carbohydrate epitopes. Vaccine. 2011 Nov. 8; 29(48):8802-26). These are referred to herein as “tumor-associated carbohydrate antigens” or “TACAs.” TACAs include, but are not limited to mucin-related antigens [including, but not limited to Tn, Sialyl Tn (STn) and Thomsen-Friedenreich antigen], blood group Lewis related antigens [including, but not limited to Lewis^{Y} (Le^{Y}), Lewis^{X} (Le^{X}), Sialyl Lewis^{X} (SLe^{X}) and Sialyl Lewis^{A} (SLe^{A})], glycosphingolipid-related antigens [including, but not limited to Globo H, stage-specific embryonic antigen-3 (SSEA-3) and glycosphingolipids comprising sialic acid], ganglioside-related antigens [including, but not limited to gangliosides GD2, GD3, GM2, fucosyl GM1 and Neu5GcGM3] and polysialic acid-related antigens. Many of such antigens are described in International Patent Application No. PCT/US2011/021387, the contents of which are herein incorporated by reference in their entirety.

In some embodiments, TACA targets of the present invention include Lewis blood group antigens. Lewis blood group antigens comprise a fucose residue linked to GlcNAc by an α1-3 linkage or an α1-4 linkage. They may be found on both glycolipids and glycoproteins. Lewis blood group antigens may be found in the body fluid of individuals that are secretors of these antigens. Their appearance on red cells is due to absorption of Lewis antigens from the serum by the red cells.

In some embodiments, TACA targets of the present invention comprise Le^{Y}. Le^{Y} (also known as CD174) is made up of Galβ1,4GlcNAC comprising α1,2- as well as α1,3-linked fucose residues yielding the Fucα(1,2)Galβ(1,4)Fucα(1,3)GlcNAc epitope. It is synthesized from the H antigen by α1,3 fucosyltransferases which attach the α1,3 fucose to the GlcNAc residue of the parent chain. Le^{Y} may be expressed in a variety of cancers including, but not limited to ovarian, breast, prostate, colon, lung and epithelial. Due to its low expression level in normal tissues and elevated expression level in many cancers, the Le^{Y} antigen is an attractive target for therapeutic antibodies.

In some embodiments, TACA targets of the present invention comprise Le^{X}. Le^{X} comprises the epitope Galβ1-4(Fucα1-3)GlcNAcβ-R. It is also known as CD15 and stage-specific embryonic antigen-1 (SSEA-1). This antigen was first recognized as being immunoreactive with sera taken from a mouse subjected to immunization with F9 teratocarcinoma cells. Le^{X} was also found to correlate with embryonic development at specific stages. It is also expressed in a variety of tissues both in the presence and absence of cancer, but can also be found in breast and ovarian cancers where it is only expressed by cancerous cells.

In some embodiments, TACA targets of the present invention comprise SLe^{A} and/or SLe^{X}. SLe^{A} and SLe^{X} comprise the structures [Neu5Acα2-3Galβ1-3(Fucα1-4)GlcNAcβ-R] and [Neu5Acα2-3Galβ1-4(Fucα1-3)GlcNAcβ-R] respectively. Their expression is upregulated in cancer cells. The presence of these antigens in serum correlates with malignancy and poor prognosis. SLe^{X} is mostly found as a mucin terminal epitope. It is expressed in a number of different cancers including breast, ovarian, melanoma, colon, liver, lung and prostate. In some embodiments of the present invention, SLe^{A} and SLe^{X} targets comprise Neu5Gc (referred to herein as GcSLe^{A} and GcSLe^{X}, respectively).

In some cases, cancer-related targets of the invention may include mucins. Ishida et al demonstrate that interaction of MUC2 with dendritic cells (with anti-tumor activity) leads to dendritic cell apoptosis (Ishida, A. et al., 2008. Proteomics. 8: 3342-9, the contents of which are herein incorporated by reference in their entirety). In some aspects, the present invention provided anti-mucin antibodies to prevent dendritic cell apoptosis and support anti-tumor activity.

In some embodiments, TACA targets of the present invention comprise glycolipids and/or epitopes present on glycolipids, including, but not limited to glycosphingolipids. Glycosphingolipids comprise the lipid ceramide linked to a glycan by the ceramide hydroxyl group. On the cell membrane, glycosphingolipids form clusters referred to as “lipid rafts”.

In some embodiments, TACA targets of the present invention comprise Globo H. Globo H is a cancer-related glycosphingolipid first identified in breast cancer cells. The glycan portion of Globo H comprises Fucα(1-2)Galβ(1-3)GalNAcβ(1-3)Galα(1-4)Galβ(1-4)Glcβ(1). Although found in a number of normal epithelial tissues, Globo H has been identified in association with many tumor tissues including, but not limited to, small cell lung, breast, prostate, lung, pancreatic, gastric, ovarian and endometrial tumors.

In some embodiments, cancer-related glycosphingolipid targets of the present invention include gangliosides. Gangliosides are glycosphingolipids comprising sialic acid. According to ganglioside nomenclature, G is used as an abbreviation for ganglioside. This abbreviation is followed by the letters M, D or T referring to the number of sialic acid residues attached (1, 2 or 3 respectively). Finally the numbers 1, 2 or 3 are used to refer to the order of the distance each migrates when analyzed by thin layer chromatography (wherein 3 travels the greatest distance, followed by 2 and then 1). Gangliosides are known to be involved in cancer-related growth and metastasis and are expressed on the cell surface of tumor cells. Gangliosides expressed on tumor cells include, but are not limited to GD2, GD3, GM2 and fucosyl GM1 (also referred to herein as Fuc-GM1). In some embodiments of the present invention, glycan-interacting antibodies are directed toward GD3. GD3 is a regulator of cell growth. In some embodiments, GD3-directed antibodies are used to modulate cell growth and/or angiogenesis. In some embodiments, GD3-directed antibodies are used to modulate cell attachment. GD3 associated with some tumor cells may comprise 9-O-acetylated sialic acid residues (Mukherjee, K. et al., 2008. J Cell Biochem. 105: 724-34 and Mukherjee, K. et al., 2009. Biol Chem. 390: 325-35, the contents of each of which are herein incorporated by reference in their entirety). In some cases, antibodies of the invention are selective for 9-O-acetylated sialic acid residues. Some antibodies may be specific for 9-O-acetylated GD3s. Such antibodies may be used to target tumor cells expressing 9-O-acetylated GD3. In some embodiments of the present invention, glycan interacting antibodies are directed toward GM2. In some embodiments, GM2-directed antibodies are used to modulate cell to cell contact. In some embodiments, ganglioside targets of the present invention comprise Neu5Gc. In some embodiments, such targets may include a GM3 variant comprising Neu5Gc (referred to herein as GcGM3). The glycan component of GcGM3 is Neu5Gcα2-3Galβ3-4Glc. GcGM3 is a known component of tumor cells (Casadesus, A. V. et al., 2013. Glycoconj J. 30(7):687-99, the contents of which are herein incorporated by reference in their entirety).

In some embodiments, tumor-associated carbohydrate antigens of the present invention comprise Neu5Gc.

### Immunogenic Hosts

In some embodiments, glycan-interacting antibodies of the present invention may be developed through the use of non-human animals as hosts for immunization, referred to herein as “immunogenic hosts”. In some embodiments, immunogenic hosts are mammals. In some embodiments, immunogenic hosts are transgenic knockout mice. Antigens comprising target sites and/or epitope targets of glycan-interacting antibodies may be used to contact immunogenic hosts in order to stimulate an immune response and produce antibodies in the immunogenic host that specifically bind the target sites and/or epitope targets present on the antigens introduced.

According to some methods of the present invention, the development of anti-STn antibodies may comprise immunizing mice that have had the Cmah gene disrupted. Such mutations may result in more human-like physiology in that Neu5Gc, the immunogenic, non-human form of sialic acid, is no longer produced in such mice. Also provided is a Cmah^{-/-} myeloma cell for producing a hybridoma that is free of Neu5Gc expression, for production of a GcSTn monoclonal antibody either by reducing the amount of recoverable anti-GcSTn or the hybridoma will begin to die due to antibody binding back to the hybridoma. Other genes can be knocked out in the background of Cmah^{-/-} myeloma cells. For example, the alpha1,3-galactosyltransferase gene, which encodes an enzyme critical for the formation of an epitope highly-immunogenic to humans (Chung, C. H. et al., Cetuximab-induced anaphylaxis and IgE specific for galactose-alpha-1,3-galactose. N Engl J Med. 2008 Mar. 13; 358(11):1109-17), can be knocked out in the background of Cmah^{-/-} myeloma cells.

According to other methods of the present invention, wild type mice may be used for immunization. Such methods may sometimes be favorable for the production of antibodies that interact with AcSTn or pan-STn epitopes. In some cases, immune responses in wild type mice may be more robust.

Antibodies produced through immunization may be isolated from serum of the immunogenic hosts. Antibody producing cells from the immunogenic hosts may also be used to generate cell lines that produce the desired antibody. In some embodiments, screening for antibodies and/or antibody producing cells from the immunogenic host may be carried out through the use of enzyme-linked immunosorbent assays (ELISAs) and/or glycan arrays.

### Adjuvants

Immunization of immunogenic hosts with antigens described herein may comprise the use of one or more adjuvants. Adjuvants may be used to elicit a higher immune response in such immunogenic hosts. As such, adjuvants used according to the present invention may be selected based on their ability to affect antibody titers.

In some embodiments, water-in-oil emulsions may be useful as adjuvants. Water-in-oil emulsions may act by forming mobile antigen depots, facilitating slow antigen release and enhancing antigen presentation to immune components. Water-in-oil emulsion-based adjuvants include. Freund's adjuvant may be used as complete Freund's adjuvant (CFA,) which comprises mycobacterial particles that have been dried and inactivated, or incomplete Freund's adjuvant (IFA,) lacking such particles, may be used. Other water-in-oil-based adjuvants may include EMULSIGEN® (MVP Technologies, Omaha, NE) EMULSIGEN® comprises micron sized oil droplets that are free from animal-based components. It may be used alone or in combination with other adjuvants, including, but not limited to aluminum hydroxide and CARBIGEN™ (MVP Technologies, Omaha, NE).

In some embodiments, TITERMAX® adjuvant may be used. TITERMAX® is another water-in-oil emulsion comprising squalene as well as sorbitan monooleate 80 (as an emulsifier) and other components. In some cases, TITERMAX® may provide higher immune responses, but with decreased toxicity toward immunogenic hosts.

Immunostimmulatory oligonucleotides may also be used as adjuvants. Such adjuvants may include CpG oligodeoxynucleotide (ODN). CpG ODNs are recognized by Toll-like receptor 9 (TLR9) leading to strong immunostimulatory effects. Type C CpG ODNs induce strong IFN-α production from plasmacytoid dendritic cell (pDC) and B cell stimulation as well as IFN-γ production from T-helper (T_{H}) cells. CpG ODN adjuvant has been shown to significantly enhance pneumococcal polysaccharide (19F and type 6B)-specific IgG2a and IgG3 in mice. CpG ODN also enhanced antibody responses to the protein carrier CRM197, particularly CRM197-specific IgG2a and IgG3 (Chu et al., Infection Immunity 2000, vol 68(3):1450-6). Additionally, immunization of aged mice with pneumococcal capsular polysaccharide serotype 14 (PPS14) combined with a CpG-ODN restored IgG anti-PPS14 responses to young adult levels (Sen et al., Infection Immunity, 2006, 74(3):2177-86). CpG ODNs used according to the present invention may include class A, B or C ODNs. In some embodiments, ODNs may include any of those available commercially, such as ODN-1585, ODN-1668, ODN-1826, ODN-2006, ODN-2007, ODN-2216, ODN-2336, ODN-2395 and/or ODN-M362, each of which may be purchased, for example, from InvivoGen, (San Diego, CA). In some cases, ODN-2395 may be used. ODN-2395 is a class C CpG ODN that specifically stimulated human as well as mouse TLR9. These ODNs comprise phosphorothioate backbones and CpG palindromic motifs.

In some embodiments, immune stimulating complexes (ISCOMs) may be used as adjuvants. ISCOMs are spherical open cage-like structures (typically 40 nm in diameter) that are spontaneously formed when mixing together cholesterol, phospholipids and Quillaia saponins under a specific stoichiometry. ISCOM technology is proven for a huge variety of antigens from large glycoproteins such as gp340 from Epstein-Barr virus (a 340 kDa antigen consisting of 80% carbohydrates) down to carrier-conjugated synthetic peptides and small haptens such as biotin. Some ISCOMs are capable of generating a balanced immune response with both T_{H1} and T_{H2} characteristics. Immune response to ISCOMs is initiated in draining lymph nodes, but is efficiently relocated to the spleen, which makes it particularly suitable for generating monoclonal antibodies as well. In some embodiments, the ISCOM adjuvant AbISCO-100 (Isconova, Uppsala, Sweden) may used. AbISCO-100 is a saponin-based adjuvant specifically developed for use in immunogenic hosts, such as mice, that may be sensitive to other saponins.

According to embodiments of the present invention, adjuvant components of immunization solutions may be varied in order to achieve desired results. Such results may include modulating the overall level of immune response and/or level of toxicitiy in immunogenic hosts.

### Glycan Arrays

In some embodiments, glycan-interacting antibodies of the present invention may be developed through the use of glycan arrays. As used herein, the term “glycan array” refers to a tool used to identify agents that interact with any of a number of different glycans linked to the array substrate. In some embodiments, glycan arrays comprise a number of chemically-synthesized glycans, referred to herein as “glycan probes”. In some embodiments, glycan arrays comprise at least 2, at least 5, at least 10, at least 20, at least 30, at least 40, at least 50, at least 60, at least 70, at least 80, at least 90, at least 100, at least 150, at least 350, at least 1000 or at least 1500 glycan probes. In some embodiments, glycan arrays may be customized to present a desired set of glycan probes. In some embodiments, glycan probes may be attached to the array substrate by a linker molecule. Such linkers may comprise molecules including, but not limited to —O(CH₂)₂CH₂)NH₂ and O(CH₂)₃NHCOCH₂(OCH₂CH₂)₆NH₂.

In some embodiments, a glycan array has more than 70 chemically-synthesized glycans, most of which are presented as Neu5Ac and Neu5Gc-containing glycan pairs. Some examples of glycan probes may include: Neu5Ac-α-2-6-GalNAc (AcSTn); Neu5Gc-α-2-6-GalNAc (GcSTn); Neu5,9Ac2-α-2,6-GalNAc; Neu9Ac5Gc-α-2,6-GalNAc, and GalNAc (Tn). The antibody binding specificity to AcSTn vs. GcSTn can be determined using the array or other methods of determining specificity known in the art. In addition, the binding profile of antibodies to O-acetylated STn can be determined. The loss of O-acetylation on STn is relevant to cancer as cancer-associated expression correlates with increased STn recognition by antibodies (Ogata, S. et al., Tumor-associated sialylated antigens are constitutively expressed in normal human colonic mucosa. Cancer Res. 1995 May 1; 55(9):1869-74) In some cases, glycan arrays may be used to determine recognition of STn vs. Tn.

### Antibody Fragment Display Library Screening Techniques

In some embodiments, antibodies of the present invention may be produced and/or optimized using high throughput methods of discovery. Such methods may include any of the display techniques (e.g. display library screening techniques) disclosed in International Patent Application No. WO2014074532, the contents of which are herein incorporated by reference in their entirety. In some embodiments, synthetic antibodies may be designed, selected or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the complementarity determining regions (CDRs). In some cases, scFvs are expressed as fusion proteins, linked to viral coat proteins (e.g. the N-terminus of the viral pIII coat protein). V_{L} chains may be expressed separately for assembly with V_{H} chains in the periplasm prior to complex incorporation into viral coats. Precipitated library members may be sequenced from the bound phage to obtain cDNA encoding desired scFvs. Such sequences may be directly incorporated into antibody sequences for recombinant antibody production, or mutated and utilized for further optimization through in vitro affinity maturation.

### Development of Cytotoxic Antibodies

In some embodiments, antibodies of the present invention may be capable of inducing antibody-dependent cell-mediated cytotoxicity (ADCC) and/or antibody-dependent cell phagocytosis (ADCP). ADCC is an immune mechanism whereby cells are lysed as a result of immune cell attack. Such immune cells may include CD56+ cells, CD3− natural killer (NK) cells, monocytes and neutrophills (Strohl, W. R. Therapeutic Antibody Engineering. Woodhead Publishing, Philadelphia PA. 2012. Ch. 8, p 186, the contents of which are herein incorporated by reference in their entirety).

In some cases, antibodies of the present invention may be engineered to comprise a given isotype depending on whether or not ADCC or ADCP is desired upon antibody binding. Such antibodies, for example, may be engineered according to any of the methods disclosed by Alderson, K. L. et al., J Biomed Biotechnol. 2011. 2011:379123). In the case of mouse antibodies, different isotypes of antibodies are more effective at promoting ADCC. IgG2a, for example, is more effective at inducing ADCC than is IgG2b. Some antibodies of the present invention, comprising mouse IgG2b antibodies may be reengineered to comprise IgG2a antibodies. Such reengineered antibodies may be more effective at inducing ADCC upon binding cell-associated antigens.

In some embodiments, genes encoding variable regions of antibodies developed according to methods of the present invention may be cloned into mammalian expression vectors encoding human Fc regions. Such Fc regions may comprise Fc regions from human IgG1κ. IgG1κ Fe regions may comprise amino acid mutations known to enhance Fc-receptor binding and antibody-dependent cell-mediated cytotoxicity (ADCC).

In some embodiments, antibodies of the invention may be developed for antibody-drug conjugate (ADC) therapeutic applications. ADCs are antibodies in which one or more cargo (e.g. therapeutic compounds or cytotoxic agents) are attached [e.g. directly or via linker (e.g. a cleavable linker or a non-cleavable linker)]. ADCs are useful for delivery of such therapeutic compounds or cytotoxic agents to one or more target cells or tissues (Panowski, S. et al., 2014. mAbs 6:1, 34-45). In some cases, ADCs may be designed to bind to a surface antigen on a targeted cell. Upon binding, the entire antibody-antigen complex may be internalized and directed to a cellular lysosome. ADCs may then be degraded, releasing the bound cargo. Where the cargo is a cytotoxic agent, the target cell will be killed or otherwise disabled. Cytotoxic agents may include, but are not limited to cytoskeletal inhibitors [e.g. tubulin polymerization inhibitors such as maytansines or auristatins (e.g. monomethyl auristatin E [MMAE] and monomethyl auristatin F [MMAF])] and DNA damaging agents (e.g. DNA polymerization inhibitors such as calcheamicins and duocarmycins).

In some embodiments, antibodies of the invention may be tested for their ability to promote cell death when developed as ADCs. Cell viability assays may be performed in the presence and absence of secondary antibody-drug conjugates. Antibodies with potent cell growth inhibition may then be used to design direct antibody-drug conjugates (ADCs). The use of such secondary antibody-drug conjugates in cell-based cytotoxic assays may allow for quick pre-screening of many ADC candidates. Based on such assays, an unconjugated antibody candidate is directly added to cells in the presence of a secondary antibody that is conjugated to one or more cytotoxic agents (referred to herein as a 2°ADC). Internalization of the antibody/2°ADC complex into cells that express a high density of the targeted antigen can achieve a dose-dependent drug release within the cells, causing a cytotoxic effect to kill the cells (e.g., tumor cells), while cells expressing a low density of the targeted antigen are not affected (e.g., normal cells).

ADCs of the invention may be designed to target cancer cells. Such ADCs may comprise antibodies directed to one or more tumor-associated carbohydrate antigen (TACA). In some cases, ADCs of the invention comprise anti-STn antibodies.

### Development of Chimeric Antigen Receptors

In some embodiments, antibody sequences of the invention may be used to develop a chimeric antigen receptor (CAR). CARs are transmembrane receptors expressed on immune cells that facilitate recognition and killing of target cells (e.g. tumor cells). CARs typically comprise three basic parts. These include an ectodomain (also known as the recognition domain), a transmembrane domain and an intracellular (signaling) domain. Ectodomains facilitate binding to cellular antigens on target cells, while intracellular domains typically comprise cell signaling functions to promote the killing of bound target cells. Further, they may have an extracellular domain with one or more antibody variable domains described herein or fragments thereof. CARs of the invention also include a transmembrane domain and cytoplasmic tail. CARs may be designed to include one or more segments of an antibody, antibody variable domain and/or antibody CDR, such that when such CARs are expressed on immune effector cells, the immune effector cells bind and clear any cells that are recognized by the antibody portions of the CARs.

Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

CARs engineered to target tumors may have specificity for one or more tumor associated carbohydrate antigens (TACAs). In some embodiments, ectodomains of these CARs may comprise one or more antibody variable domains or a fragment thereof. In some embodiments, CARs are expressed in T cells, and may be referred to as “CAR-engineered T cells” or “CAR-Ts”. CAR-Ts may be engineered with CAR ectodomains having one or more antibody variable domains.

### Structural Features of Chimeric Antigen Receptors

With gene-transfer technology, T cells can be engineered to stably express antibodies on their surface, conferring a desired antigen specificity. Chimeric antigen receptors (CARs) combine an antigen-recognition domain of a specific antibody with an intracellular domain of the CD3-zeta chain or FcγRI protein having T cell activating properties into a single chimeric fusion protein. CAR technology provides MHC-unrestricted recognition of target cells by T cells. Removal of the MHC restriction of T cells facilitates the use of these molecules in any patient, and also, in both CD8⁺ and CD4⁺ T cells, usually restricted to MHC class I or II epitopes, respectively. The use of Ab-binding regions allows T cells to respond to epitopes formed not only by protein, but also carbohydrate and lipid. This chimeric receptor approach is especially suited to immunotherapy of cancer, being able to bypass many of the mechanisms by which tumors avoid immunorecognition, such as MHC down-regulation, lack of expression of costimulatory molecules, CTL resistance, and induction of T cell suppression, and where the use of both CD8⁺ CTL and CD4⁺ T cells are best combined for optimum antitumor efficacy. This approach has been demonstrated to be applicable to a wide range of tumor antigens, in addition to viruses such as HIV (Finney, et al., J. Immunology, 2004, 172:104-113).

Although chimeric antigen receptors can trigger T-cell activation in a manner similar to that of endogenous T-cell receptors, in practice, the clinical application of CAR technology has been impeded by inadequate in vivo expansion of chimeric antigen receptor T cells. For example, first generation CARs included as their signaling domain the cytoplasmic region of the CD3ζ or Fc receptor γ chain. These first generation CARs were tested in phase I clinical studies in patients with ovarian cancer, renal cancer, lymphoma, and neuroblastoma, and were found to induce modest responses, effectively redirecting T cell cytotoxicity but failing to enable T cell proliferation and survival upon repeated antigen exposure. The prototypes for second generation CARs involved receptors encompassing both CD28 and CD3ζ, and second generation CARs have been tested for treatment of B cell malignancies and other cancers (Sadelain, et al., (2009) Current Opinion in Immunology, 21(2):215-223). Thus, CARs have rapidly expanded into a diverse array of receptors with different functional properties.

More recently, it was discovered that CAR-mediated T-cell responses can be enhanced with the addition of a costimulatory domain. In preclinical models, the inclusion of the CD137 (4-1BB) signaling domain was found to significantly increase antitumor activity and in vivo persistence of chimeric antigen receptors as compared with inclusion of the CD3-zeta chain alone (Porter, et al., N. Engl. J. Med. 2011, 365:725-733).

Thus, in some embodiments of the present disclosure, antibody sequences of the invention may be used to develop a chimeric antigen receptor (CAR). In some embodiments, CARs are transmembrane receptors expressed on immune cells that facilitate recognition and killing of target cells (e.g. tumor cells).

In many cancers, tumor-specific antigens for targeting have not been defined, but in B-cell neoplasms, CD19 is an attractive target. Expression of CD19 is restricted to normal and malignant B cells and B-cell precursors. A pilot clinical trial of treatment with autologous T cells expressing an anti-CD19 chimeric antigen receptor (CART19) was performed in patients with advanced, p53-deficient chronic lymphoid leukemia (CLL). The generation of a CD19-specific immune response in bone marrow was demonstrated by temporal release of cytokines and ablation of leukemia cells that coincided with peak infiltration of chimeric antigen receptor T cells. (Porter, et al., N. Engl. J. Med. 2011, 365:725-733).

Further structural features of CARs may include any of those disclosed in several PCT Publications assigned to City of Hope and having the common inventor Michael Jensen. For example, PCT Publication WO 00/23573 describes genetically engineered, CD20-specific redirected T cells expressing a cell surface protein having an extracellular domain comprising a receptor specific for CD20, an intracellular signaling domain, and a transmembrane domain. Use of such cells for cellular immunotherapy of CD20⁺ malignancies and for abrogating any untoward B cell function. In one embodiment, the cell surface protein is a single chain FvFc:ζ receptor where Fv designates the VH and VL chains of a single chain monoclonal antibody to CD20 linked by peptide, Fc represents a hinge-CH2-CH3 region of a human IgG1, and ζ represents the intracellular signaling domain of the zeta chain of human CD3. A method of making a redirected T cell expressing a chimeric T cell receptor by electroporation using naked DNA encoding the receptor. Similarly, PCT Publication WO 02/077029 describes genetically engineered, CD19-specific redirected immune cells expressing a cell surface protein having an extracellular domain comprising a receptor which is specific for CD19, an intracellular signaling domain, and a transmembrane domain. Use of such cells for cellular immunotherapy of CD19⁺ malignancies and for abrogating any untoward B cell function. In one embodiment, the immune cell is a T cell and the cell surface protein is a single chain svFvFc:ζ receptor where scFc designates the VH and VL chains of a single chain monoclonal antibody to CD19, Fc represents at least part of a constant region of an IgG1, and zeta represents the intracellular signaling domain of the T cell antigen receptor complex zeta chain (zeta chain of human CD3). The extracellular domain scFvFc and the intracellular domain zeta are linked by a transmembrane domain such as the transmembrane domain of CD4. A method of making a redirected T cell expressing a chimeric T cell receptor by electroportion using naked DNA encoding the receptor. These chimeric antigen receptors have the ability, when expressed in T cells, to redirect antigen recognition based on the monoclonal antibody's specificity. The design of scFvFc: receptors with target specificities for tumor cell-surface epitopes is a conceptually attractive strategy to generate antitumor immune effector cells for adoptive therapy as it does not rely on pre-existing anti-tumor immunity. These receptors are “universal” in that they bind antigen in a MHC independent fashion, thus, one receptor construct can be used to treat a population of patients with antigen positive tumors. City of Hope PCT Publications WO 02/088334, WO 2007/059298 and WO 2010/065818 describe “zetakines” comprised of an extracellular domain comprising a soluble receptor ligand linked to a support region capable of tethering the extracellular domain to a cell surface, a transmembrane region and an intracellular signalling domain. Zetakines, when expressed on the surface of T lymphocytes, direct T cell activity to those specific cells expressing a receptor for which the soluble receptor ligand is specific.

Additional features of CARs may include any of those disclosed in two PCT Publications assigned to University of Texas and having a common inventor Lawrence Cooper. PCT Publication No. WO 2009/091826 describes compositions comprising a human CD19-specific chimeric T cell receptor (or chimeric antigen receptor, CAR) polypeptide (designated hCD19CAR) comprising an intracellular signaling domain, a transmembrane domain and an extracellular domain, the extracellular domain comprising a human CD 19 binding region. In another aspect, the CD 19 binding region is an F(ab′)2, Fab′, Fab, Fv or scFv. The intracellular domain may comprise an intracellular signaling domain of human CD3ζ and may further comprise human CD28 intracellular segment. In certain aspects the transmembrane domain is a CD28 transmembrane domain. PCT Publication No. WO 2013/074916 describes methods and compositions for immunotherapy employing CAR⁺ T cells genetically modified to eliminate expression of T cell receptor and/or HLA. In particular embodiments, the T cell receptor-negative and/or HLA-negative T cells are generated using zinc finger nucleases, for example. The CAR⁺ T cells from allogeneic healthy donors can be administered to any patient without causing graft versus host disease (GVHD), acting as universal reagents for off-the-shelf treatment of medical conditions such as cancer, autoimmunity, and infection.

PCT Publication WO 2011/041093 assigned to the U.S. Department of Health and Human Services describes anti-vascular endothelial growth factor receptor-2 chimeric antigen receptors comprising an antigen binding domain of a KDR-1121 or DC101 antibody, an extracellular hinge domain, a T cell receptor transmembrane domain, and an intracellular T cell receptor signaling domain, and their use in the treatment of cancer.

PCT Publications WO 2012/079000 and WO 2013/040557, the contents of each of which are herein incorporated by reference in their entirety, are assigned to University of Pennsylvania and share the common inventor Carl H. June; these publications describe CARs comprising an antigen binding domain, a transmembrane domain, a costimulatory signaling region, and a CD3 zeta signaling domain, and methods for generating RNA Chimeric Antigen Receptor (CAR) transfected T cells, respectively.

PCT Publication WO2013/126712, also assigned to University of Pennsylvania and sharing the common inventor Carl H. June, describes compositions and methods for generating a persisting population of T cells exhibiting prolonged exponential expansion in culture that is ligand independent and independent of the addition of exogenous cytokines or feeder cells, which are useful for the treatment of cancer. In some embodiments, the antigen binding domain is an anti-cMet binding domain. In some embodiments, the antigen binding domain is an anti-mesothelin binding domain. In some embodiments, the antigen binding domain is an anti-CD 19 binding domain. The hinge domain is IgG4, the transmembrane domain is a CD28 transmembrane domain. In some embodiments, the costimulatory signaling region is a CD28 signaling region. Also provided is a vector comprising a nucleic acid sequence encoding a chimeric antigen receptor (CAR), and the CAR comprising an antigen binding domain, a hinge domain, a transmembrane domain, a costimulatory signaling region, and a CD3 zeta signaling domain.

PCT Publication WO 2014/039513 assigned to University of Pennsylvania describes compositions and methods for inhibiting one or more diacylglycerol kinase (DGK) isoform in a cell in order to enhance the cytolytic activity of the cell. The cells may be used in adoptive T cell transfer in which, the cell is modified to express a chimeric antigen receptor (CAR). Inhibition of DGK in T cells used in adoptive T cell transfer increases cytolytic activity of the T cells and thus may be used in the treatment of a variety of conditions, including cancer, infection, and immune disorders.

PCT Publication WO 2014/055771 assigned to University of Pennsylvania describes compositions and methods for treating ovarian cancer. Specifically, the invention relates to administering a genetically modified T cell having alpha-folate receptor (FR-alpha) binding domain and CD27 costimulatory domain to treat ovarian cancer. In one embodiment, the FR-alpha binding domain is said to be fully human, thereby preventing a host immune response.

In some embodiments, CARs of the invention may be engineered to target tumors. Such CARs may have specificity for one or more TACAs. In some case, ectodomains of these CARs may comprise one or more antibody variable domain presented herein or a fragment thereof. In some embodiments, CARs of the invention are expressed in T cells, referred to herein as “CAR-engineered T cells” or “CAR-Ts”. CAR-Ts may be engineered with CAR ectodomains having one or more antibody variable domain presented herein.

### Multispecific Antibodies

In some embodiments, antibodies of the present invention may bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

### Bispecific Antibodies

Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. One common application for this technology is in cancer immunotherapy, where BsMAbs are engineered to simultaneously bind to a cytotoxic cell (using a receptor like CD3) and a target like a tumor cell to be destroyed.

Bispecific antibodies may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally binds to a cell that expresses Fc receptors, like a mactrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

A bispecific, single-chain antibody Fv fragment (Bs-scFv) was successfully used to kill cancer cells. Some human cancers are caused by functional defects in p53 that are restored by gene therapy with wild-type p53. Weisbart, et al., describe the construction and expression of a bispecific single-chain antibody that penetrates living colon cancer cells, binds intracellular p53, and targets and restores its wild type function (Weisbart, et al., Int. J. Oncol. 2004 October; 25(4):1113-8; and Weisbart, et al., Int. J. Oncol. 2004 December; 25(6):1867-73). In these studies, a bispecific, single-chain antibody Fv fragment (Bs-scFv) was constructed from (i) a single-chain Fv fragment of mAb 3E10 that penetrates living cells and localizes in the nucleus, and (ii) a single-chain Fv fragment of a non-penetrating antibody, mAb PAb421 that binds the C-terminal of p53. PAb421 binding restores wild-type functions of some p53 mutants, including those of SW480 human colon cancer cells. The Bs-scFv penetrated SW480 cells and was cytotoxic, suggesting an ability to restore activity to mutant p53. COS-7 cells (monkey kidney cells with wild-type p53) served as a control since they are unresponsive to PAb421 due to the presence of SV40 large T antigen that inhibits binding of PAb421 to p53. Bs-scFv penetrated COS-7 cells but was not cytotoxic, thereby eliminating non-specific toxicity of Bs-scFv unrelated to binding p53. Fv fragments alone were not cytotoxic, indicating that killing was due to transduction of p53. A single mutation in CDR1 of PAb421 VH eliminated binding of the Bs-scFv to p53 and abrogated cytotoxicity for SW480 cells without altering cellular penetration, further supporting the requirement of PAb421 binding to p53 for cytotoxicity (Weisbart, et al., Int. J. Oncol. 2004 October; 25(4):1113-8; and Weisbart, et al., Int. J. Oncol. 2004 December; 25(6):1867-73).

In some embodiments, antibodies of the present invention may be diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs. 2010. January-February; 2(1):77-83).

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications, and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs. 2010. January-February; 2(1):77-83).

Also included are maxibodies (bivalent scFV fused to the amino terminus of the Fc (CH2-CH3 domains) of IgG.

Bispecific T-cell-engager (BiTE) antibodies are designed to transiently engage cytotoxic T-cells for lysis of selected target cells. The clinical activity of BiTE antibodies corroborates findings that ex vivo expanded, autologous T-cells derived from tumor tissue, or transfected with specific T-cell receptors, have shown therapeutic potential in the treatment of solid tumors. While these personalized approaches prove that T-cells alone can have considerable therapeutic activity, even in late-stage cancer, they are cumbersome to perform on a broad basis. This is different for cytotoxic T-lymphocyte antigen 4 (CTLA-4) antibodies, which facilitate generation of tumor-specific T-cell clones, and also for bi- and tri-specific antibodies that directly engage a large proportion of patients' T-cells for cancer cell lysis. The potential of global T-cell engagement for human cancer therapy by T-cell-engaging antibodies is under active investigation (Baeuerle P A, et al., Current Opinion in Molecular Therapeutics. 2009, 11(1):22-30).

Third generation molecules include “miniaturized” antibodies. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE and possibly multiple sclerosis, although these projects remain in the earliest stages of clinical testing. (Nelson, A. L., MAbs. 2010. January-February; 2(1):77-83).

Genmab is researching application of their “Unibody” technology, in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promoteintracellular signaling complex formation. These contentions are, however, largely supported by laboratory, rather than clinical, evidence. Biotecnol is also developing a “miniaturized” mAb, CAB051, which is a “compacted” 100 kDa anti-HER2 antibody in preclinical research (Nelson, A. L., MAbs. 2010. January-February; 2(1):77-83).

Recombinant therapeutics composed of single antigen-binding domains have also been developed, although they currently account for only 4% of the clinical pipeline. These molecules are extremely small, with molecular weights approximately one-tenth of those observed for full-sized mAbs. Arana and Domantis engineer molecules composed of antigen-binding domains of human immunoglobulin light or heavy chains, although only Arana has a candidate in clinical testing, ART-621, an anti-TNFα molecule in Phase 2 study for the treatment of psoriasis and rheumatoid arthritis. Ablynx produces “nanobodies” derived from the antigen-binding variable heavy chain regions (V_{HH}S) of heavy chain antibodies found in camels and llamas, which lack light chains. Two Ablynx anti-von Willebrand Factor nanobodies have advanced to clinical development, including ALX-0081, in Phase 2 development as an intravenous therapy to prevent thrombosis in patients undergoing percutaneous coronary intervention for acute coronary syndrome, and ALX-0681, a Phase 1 molecule for subcutaneous administration intended for both patients with acute coronary syndrome and thrombotic thrombocytopenic purpura (Nelson, A. L., MAbs. 2010. January-February; 2(1):77-83).

### Development of Multispecific Antibodies

In some embodiments, antibody sequences of the invention may be used to develop multispecific antibodies (e.g., bispecific, trispecific, or of greater multispecificity). Multispecific antibodies can be specific for different epitopes of a target antigen of the present invention, or can be specific for both a target antigen of the present invention, and a heterologous epitope, such as a heterologous glycan, peptide or solid support material. (See, e.g., WO 93/17715; WO 92/08802; WO 91/00360; WO 92/05793; Tutt, A. et al., Trispecific F(ab)3 derivatives that use cooperative signaling via the TCR/CD3 complex and CD2 to activate and redirect resting cytotoxic T cells. J. Immunol. 1991 Jul. 1; 147(1):60-9; U.S. Pat. Nos. 4,474,893; 4,714,681; 4,925,648; 5,573,920; 5,601,819; and Kostelny, S. A. et al., Formation of a bispecific antibody by the use of leucine zippers. J. Immunol. 1992 Mar. 1; 148(5):1547-53); U.S. Pat. No. 5,932,448.

Disclosed and claimed in PCT Publication WO2014144573 to Memorial Sloan-Kettering Cancer Center are multimerization technologies for making dimeric multispecific binding agents (e.g., fusion proteins comprising antibody components) with improved properties over multispecific binding agents without the capability of dimerization.

Disclosed and claimed in PCT Publication WO2014144357 to Merck Patent GMBH are tetravalent bispecific antibodies (TetBiAbs), and methods of making and methods of using TetBiAbs for diagnostics and for the treatment of cancer or immune disorders. TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

Disclosed and claimed in PCT Publication WO2014028560 to IBC Pharmaceuticals, Inc. are T cell redirecting bispecific antibodies (bsAb), with at least one binding site for a T-cell antigen and at least one binding site for an antigen on a diseased cell or pathogen, for treatment of disease. Preferably, this bsAb is an anti-CD3×anti-CD19 bispecific antibody, although antibodies against other T-cell antigens and/or disease-associated antigens may be used. The complex is capable of targeting effector T cells to induce T-cell-mediated cytotoxicity of cells associated with a disease, such as cancer, autoimmune disease or infectious disease. The cytotoxic immune response is enhanced by co-administration of interfon-based agents that comprise interferon-α, interferon-bgr; interferon-λ1, interferon-k2 or interferon-k3.

Disclosed and claimed in PCT Publication WO2013092001 to Synimmune GMBH is a bispecific antibody molecule, as well as a method for producing the same, its use and a nucleic acid molecule encoding the bispecific antibody molecule. In particular is provided an antibody molecule that is capable of mediating target cell restricted activation of immune cells.

Disclosed and claimed in PCT Publication WO2012007167 is a multispecific modular antibody specifically binding to at least a glycoepitope and a receptor of the erbB class on the surface of a tumor cell, thereby crosslinking the glycoepitope and the receptor, which antibody has apoptotic activity effecting cytolysis independent of NK cells.

Disclosed and claimed in PCT Publications WO2012048332 and WO2013055404 are meditopes, meditope-binding antibodies, meditope delivery systems, as well as a monoclonal antibody framework binding interface for meditopes, and methods for their use. Specifically, two antibody binding peptides, C-QFDLSTRRLK-C (“cQFD”; sequence identification number 1 therein; SEQ ID NO: 67 herein) and C-QYNLSSRALK-C (“cQYN”; sequence identification number 2 therein; SEQ ID NO: 68 herein) were shown to have novel mAb binding properties. Also called “meditopes,” cQFD and cQYN were shown to bind to a region of the Fab framework of the anti-EGFR mAb cetuximab and not to bind the complementarity determining regions (CDRs) that bind antigen. The binding region on the Fab framework is distinct from other framework-binding antigens, such as the superantigens Staphylococcal protein A (SpA) (Graille et al., 2000) and Peptostreptococcus magnus protein L (PpL) (Graille et al., 2001). Accordingly, one embodiment disclosed is a framework binding interface comprising a framework region of a unique murine-human antibody or functional fragment thereof that binds a cyclic meditope.

Exemplary patents and patent publications of interest are: U.S. Pat. Nos. 5,585,089; 5,693,761; and 5,693,762, all filed Jun. 7, 1995 and U.S. Pat. No. 6,180,370, all assigned to Protein Design Labs, Inc., describe methods for producing, and compositions of, humanized immunoglobulins having one or more complementarity determining regions (CDR's) and possible additional amino acids from a donor immunoglobulin and a framework region from an accepting human immunoglobulin. Each humanized immunoglobulin chain is said to usually comprise, in addition to the CDR's, amino acids from the donor immunoglobulin framework that are, e.g., capable of interacting with the CDRs to effect binding affinity, such as one or more amino acids which are immediately adjacent to a CDR in the donor immunoglobulin or those within about about 3 Å as predicted by molecular modeling. The heavy and light chains may each be designed by using any one or all of various position criteria. When combined into an intact antibody, the humanized immunoglobulins of the present invention is said to be substantially non-immunogenic in humans and retain substantially the same affinity as the donor immunoglobulin to the antigen, such as a protein or other compound containing an epitope.

U.S. Pat. No. 5,951,983, assigned to Universite Catholique De Louvain and Bio Transplant, Inc., describes a humanized antibody against T-lymphocytes. Framework regions from a human V kappa gene designated as HUM5400 (EMBL accession X55400) and from the human antibody clone Amu 5-3 (GenBank accession number U00562) are set forth therein.

U.S. Pat. No. 5,091,513, to Creative Biomolecules, Inc., describes a family of synthetic proteins having affinity for a preselected antigen. The proteins are characterized by one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L}are attached by a polypeptide linker, or 3) individuals V_{H} or V_{L} domains. The binding domains comprise linked CDR and FR regions, which may be derived from separate immunoglobulins. The proteins may also include other polypeptide sequences which function, e.g., as an enzyme, toxin, binding site, or site of attachment to an immobilization media or radioactive atom. Methods are disclosed for producing the proteins, for designing BABS having any specificity that can be elicited by in vivo generation of antibody, and for producing analogs thereof.

U.S. Pat. No. 8,399,625, to ESBATech, an Alcon Biomedical Research Unit, LLC, describes antibody acceptor frameworks and methods for grafting non-human antibodies, e.g., rabbit antibodies, using a particularly well suited antibody acceptor framework.

### Intrabodies

In some embodiments, antibodies of the present invention may be intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular traffic or by inhibiting its association with binding partners. They have been largely employed as research tools and are emerging as therapeutic molecules for the treatment of human diseases as viral pathologies, cancer and misfolding diseases. The fast growing bio-market of recombinant antibodies provides intrabodies with enhanced binding specificity, stability and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity of to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

### Development of Intrabodies

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced using methods known in the art, such as those disclosed and reviewed in: (Marasco et al., 1993 Proc. Natl. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

In some embodiments, antibody sequences are used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form a single chain antibody comprising the variable domains of the heavy and light chain joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif (SEQ ID NO: 71). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly-synthesized intrabody within the cell is affected by reducing conditions of the intracellular environment. In human clinical therapy, there are safety concerns surrounding the application of transfected recombinant DNA, which is used to achieve intrabody expression within the cell. Of particular concern are the various viral-based vectors commonly-used in genetic manipulation. Thus, one approach to circumvent these problems is to fuse protein transduction domains (PTD) to scFv antibodies, to create a ‘cell-permeable’ antibody or ‘Transbody.’ Transbodies are cell-permeable antibodies in which a protein transduction domain (PTD) is fused with single chain variable fragment (scFv) antibodies (Heng and Cao, 2005, Med Hypotheses. 64:1105-8).

Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

In one embodiment, intrabodies are used to capture a target in the nucleus, thereby preventing its activity within the nucleus. Nuclear targeting signals are engineered into such intrabodies in order to achieve the desired targeting. Such intrabodies are designed to bind specifically to a particular target domain. In another embodiment, cytosolic intrabodies that specifically bind to a target protein are used to prevent the target from gaining access to the nucleus, thereby preventing it from exerting any biological activity within the nucleus (e.g., preventing the target from forming transcription complexes with other factors).

In order to specifically direct the expression of such intrabodies to particular cells, the transcription of the intrabody is placed under the regulatory control of an appropriate tumor-specific promoter and/or enhancer. In order to target intrabody expression specifically to prostate, for example, the PSA promoter and/or promoter/enhancer can be utilized (See, for example, U.S. Pat. No. 5,919,652 issued 6 Jul. 1999).

Protein transduction domains (PTDs) are short peptide sequences that enable proteins to translocate across the cell membrane and be internalized within the cytosol, through atypical secretory and internalization pathways. There are a number of distinct advantages that a ‘Transbody’ would possess over conventional intrabodies expressed within the cell. For a start, ‘correct’ conformational folding and disulfide bond formation can take place prior to introduction into the target cell. More importantly, the use of cell-permeable antibodies or ‘Transbodies’ would avoid the overwhelming safety and ethical concerns surrounding the direct application of recombinant DNA technology in human clinical therapy, which is required for intrabody expression within the cell. ‘Transbodies’ introduced into the cell would possess only a limited active half-life, without resulting in any permanent genetic alteration. This would allay any safety concerns with regards to their application in human clinical therapy (Heng and Cao 2005, Med Hypotheses. 64:1105-8).

Intrabodies are promising therapeutic agents for the treatment of misfolding diseases, including Alzheimer's, Parkinson's, Huntington's and prion diseases, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

Exemplary Patent Publications describing intracellular antibodies or intrabodies are set forth hereinbelow, each of which is incorporated by reference in its entirety.

PCT Publication WO03014960 and U.S. Pat. No. 7,608,453 granted to Cattaneo, et al., describe an intracellular antibody capture technology method of identifying at least one consensus sequence for an intracellular antibody (ICS) comprising the steps of: creating a database comprising sequences of validated intracellular antibodies (VIDA database) and aligning the sequences of validated intracellular antibodies according to Kabat; determining the frequency with which a particular amino acid occurs in each of the positions of the aligned antibodies; selecting a frequency threshold value (LP or consensus threshold) in the range from 70% to 100%; identifying the positions of the alignment at which the frequency of a particular amino acid is greater than or equal to the LP value; and identifying the most frequent amino acid, in the position of said alignment.

PCT Publications WO0054057; WO03077945; WO2004046185; WO2004046186; WO2004046187; WO2004046188; WO2004046189; US Patent Application Publications US2005272107; US2005276800; US2005288492; US2010143939; granted U.S. Pat. Nos. 7,569,390 and 7,897,347 and granted European Patents EP1560853; and EP1166121 all assigned to the Medical Research Council and including inventors Cattaneo, et al., describe intracellular intracellular single domain immunoglobulins, and a method for determining the ability of a immunoglobulin single domain to bind to a target in an intracellular environment, as well as methods for generating intracellular antibodies.

PCT Publication WO0235237; US Patent Application Publication 2003235850 and granted European Patent EP1328814 naming Catteneo as an inventor and assigned to S.I.S.S.A. Scuola Internazionale Superiore describe a method for the in vivo identification of epitopes of an intracellular antigen.

PCT Publication WO2004046192 and European Patent EP1565558 assigned to Lay Line Genomics SPA and naming Catteneo as an inventor describe a method for isolating intracellular antibodies that disrupt and neutralize an interaction between a protein ligand x and a protein ligand γ inside a cell. Also disclosed are a method to identify a protein ligand x able to bind to a known y ligand using intracellular antibodies able to the interaction between x and y; and a method for the isolation of a set of antibody fragments against a significant proportion of the protein-protein interactions of a given cell (interactome) or against the protein interactions that constitute an intracellular pathway or network.

US Patent Application Publication 2006034834 and PCT Publication WO9914353 entitled “Intrabody-mediated control of immune reactions” and assigned to Dana Farber Cancer Institute Inc. name inventors Marasco and Mhashilkar are directed to methods of altering the regulation of the immune system, e.g., by selectively targeting individual or classes of immunomodulatory receptor molecules (IRMs) on cells comprising transducing the cells with an intracellularly expressed antibody, or intrabody, against the IRMs. In a preferred embodiment the intrabody comprises a single chain antibody against an IRM, e.g, MHC-1 molecules.

PCT Publication WO2013033420 assigned to Dana Farber Cancer Institute Inc. and Whitehead Biomedical Institute, and naming inventors Bradner, Rahl and Young describes methods and compositions useful for inhibiting interaction between a bromodomain protein and an immunoglobulin (Ig) regulatory element and downregulating expression of an oncogene translocated with an Ig locus, as well as for treating a cancer (e.g., hematological malignancy) characterized by increased expression of an oncogene which is translocated with an Ig locus. Intrabodies are generally described.

PCT Publication WO02086096 and US Patent Application Publication 2003104402 entitled “Methods of producing or identifying intrabodies in eukaryotic cells,” assigned to University of Rochester Medical Center and naming inventors Zauderer, Wei and Smith describe a high efficiency method of expressing intracellular immunoglobulin molecules and intracellular immunoglobulin libraries in eukaryotic cells using a trimolecular recombination method. Further provided are methods of selecting and screening for intracellular immunoglobulin molecules and fragments thereof, and kits for producing, screening and selecting intracellular immunoglobulin molecules, as well as the intracellular immunoglobulin molecules and fragments produced using these methods.

PCT Publication WO2013023251 assigned to Affinity Biosciences PTY LTD and naming inventors Beasley, Niven and Kiefel describes polypeptides, such as antibody molecules and polynucleotides encoding such polypeptides, and libraries thereof, wherein the expressed polypeptides that demonstrate high stability and solubility. In particular, polypeptides comprising paired VL and VH domains that demonstrate soluble expression and folding in a reducing or intracellular environment are described, wherein a human scFv library was screened, resulting in the isolation of soluble scFv genes that have identical framework regions to the human germline sequence as well as remarkable thermostability and tolerance of CDR3 grafting onto the scFv scaffold.

European Patent Application EP2314622 and PCT Publications WO03008451 and WO03097697 assigned to Esbatech AG and University of Zuerich and naming inventors Ewert, Huber, Honneger and Plueckthun describe the modification of human variable domains and provide compositions useful as frameworks for the creation of very stable and soluble single-chain Fv antibody fragments. These frameworks have been selected for intracellular performance and are thus ideally suited for the creation of scFv antibody fragments or scFv antibody libraries for applications where stability and solubility are limiting factors for the performance of antibody fragments, such as in the reducing environment of a cell. Such frameworks can also be used to identify highly conserved residues and consensus sequences which demonstrate enhanced solubility and stability.

PCT Publication WO02067849 and US Patent Application Publication 2004047891 entitled “Systems devices and methods for intrabody targeted delivery and reloading of therapeutic agents” describe systems, devices and methods for intrabody targeted delivery of molecules. More particularly, some embodiments relate to a reloadable drug delivery system, which enables targeted delivery of therapeutic agents to a tissue region of a subject, in a localized and timely manner.

PCT Publication WO2005063817 and U.S. Pat. No. 7,884,054 assigned to Amgen Inc. and naming inventors Zhou, Shen and Martin describe methods for identifying functional antibodies, including intrabodies. In particular, a homodimeric intrabody is described, wherein each polypeptide chain of the homodimer comprises an Fc region, an scFv, and an intracellular localization sequence. The intracellular localization sequence may cause the intrabody to be localized to the ER or the Golgi. Optionally, each polypeptide chain comprises not more than one scFv.

PCT Publication WO2013138795 by Vogan, et al. and assigned to Permeon Biologics Inc. describes cell penetrating compositions for delivery of intracellular antibodies and antibody-like moieties and methods for delivering them (referred to herein as “AAM moieties” or “an AAM moiety”) into a cell. Without being bound by theory, the present disclosure is based, at least in part, on the discovery that an AAM moiety can be delivered into a cell by complexing the AAM moiety with a cell penetrating polypeptide having surface positive charge (referred to herein as a “Surf+ Penetrating Polypeptide”). Examples of some applications of intraphilin technology are also provided

PCT Publication WO2010004432 assigned to the Pasteur Institute describes immunoglobulins from camelidae (camels, dromedaries, llamas and alpacas), about 50% of which are antibodies devoid of light chain. These heavy-chain antibodies interact with the antigen by the virtue of only one single variable domain, referred to as VHH(s), VHH domain(s) or VHH antibody (ies). Despite the absence of light chain, these homodimeric antibodies exhibit a broad antigen-binding repertoire by enlarging their hypervariable regions, and can act as a transbody and/or intrabody in vitro as well as in vivo, when the VHH domain is directed against an intracellular target.

PCT Publication WO2014106639 describes a method for identifying a cellular target involved in a cell phenotype by identifying an intrabody that can modify a cell phenotype and identifying a direct or indirect cellular target of the intrabody. In particular, intrabodies 3H2-1, 3H2-VH and 5H4 are capable of inhibiting the degranulation reaction in mast cells triggered by an allergic stimulus; furthermore, intrabodies 3H2-1 and 5H4 directly or indirectly targeted a protein of the ABCF1 family and C120RF4 family, respectively. These ABCF1 and C120RF4 inhibitors are said to be useful in therapy, in particular for treating allergic and/or inflammatory conditions.

PCT Publication WO0140276 assigned to Urogenesis Inc. generally describes the possibility of inhibition of STEAP (Six Transmembrane Epithelial Antigen of the Prostate) proteins using intracellular antibodies (intrabodies).

PCT Publication WO02086505 assigned to University of Manchester and US Patent Application Publication US2004115740 naming inventors Simon and Benton describe a method for the intracellular analysis of a target molecule, wherein intrabodies are said to be preferred. In one embodiment, a vector (designated pScFv-ECFP) capable of expressing an anti-MUC1 intrabody coupled to CFP is described.

PCT Publication WO03095641 and WO0143778 assigned to Gene Therapy Systems Inc. describe compositions and methods for intracellular protein delivery, and intrabodies are generally described.

PCT Publication WO03086276 assigned to Selective Genetics Inc. describes a platform technology for the treatment of intracellular infections. Compositions and methods described therein include non-target specific vectors that target infectable cells via linked ligands that bind and internalize through cell surface receptors/moieties associated with infection. The vectors comprise exogenous nucleic acid sequences that are expressed upon internalization into a target cell. Vector associated ligands and nucleic acid molecules may be altered to target different infectious agents. In addition, the invention provides methods of identifying epitopes and ligands capable of directing internalization of a vector and capable of blocking viral entry.

PCT Publication WO03062415 assigned to Erasmus University describes a transgenic organism comprising a polynucleotide construct encoding an intracellular antibody which disrupts the catalysis of the production of the xenoantigen galactose alpha 1,3 galactose and/or a polynucleotide construct which encodes an intracellular antibody which binds specifically to a retrovirus protein, such as a PERV particle protein. Cells, tissues and organs of the transgenic organism may be used in xenotransplantation.

PCT Publication WO2004099775 entitled “Means for detecting protein conformation and applications thereof” describes the use of scFv fragments as conformation-specific antibodies for specifically detecting a conformational protein state, said to have applications as sensors for following in livings cells, upon intracellular expression, the behavior of endogeneous proteins.

PCT Publication WO2008070363 assigned to Imclone Systems Inc. describes a single domain intrabody that binds to an intracellular protein or to an intracellular domain of an intracellular protein, such as Etk, the endothelial and epithelial tyrosine kinase, which is a member of the Tec family of non-receptor tyrosine kinases. Also provided is a method of inhibiting an intracellular enzyme, and treating a tumor in a patient by administering the intrabody or a nucleic acid expressing the intrabody.

PCT Publication WO2009018438 assigned to Cornell Research Foundation Inc. describes a method of identifying a protein that binds to a target molecule and has intracellular functionality, by providing a construct comprising a DNA molecule encoding the protein which binds to the target molecule, with the DNA molecule being coupled to a stall sequence. A host cell is transformed with the construct and then cultured under conditions effective to form, within the host cell, a complex of the protein whose translation has been stalled, the mRNA encoding the protein, and ribosomes. The protein in the complex is in a properly folded, active form and the complex is recovered from the cell. This method can be carried out with a cell-free extract preparation containing ribosomes instead of a host cell. The present invention also relates to a construct which includes a DNA molecule encoding a protein that binds to a target molecule and an SecM stalling sequence coupled to the DNA molecule. The DNA molecule and the SecM stalling sequence are coupled with sufficient distance between them to permit expression of their encoded protein, within the cell, in a properly folded, active form. The use of intrabodies is generally described.

PCT Publication WO2014030780 assigned to Mogam Biotech Research Institute describes a method named Tat-associated protein engineering (TAPE), for screening a target protein having higher solubility and excellent thermostability, in particular, an immunoglobulin variable domain (VH or VL) derived from human germ cells, by preparing a gene construct where the target protein and an antibiotic-resistant protein are linked to a Tat signal sequence, and then expressing this within E. coli. Also disclosed are human or engineered VH and VL domain antibodies and human or engineered VH and VL domain antibody scaffolds having solubility and excellent thermostability, which are screened by the TAPE method. Also provided is a library including random CDR sequences in the human or engineered VH or VL domain antibody scaffold screened by the TAPE method, a preparing method thereof, a VH or VL domain antibody having binding ability to the target protein screened by using the library, and a pharmaceutical composition including the domain antibody.

European Patent Application EP2422811 describes an antibody that binds to an intracellular epitope; such intrabodies comprise at least a portion of an antibody that is capable of specifically binding an antigen and preferably does not contain operable sequences coding for its secretion and thus remains within the cell. In one embodiment, the intrabody comprises a scFv. The scFv polypeptide further comprises a polypeptide linker between the VH and VL domains which enables the scFv to form the desired structure for antigen binding. Also described is a specific embodiment in which the intrabody binds to the cytoplasmic domain of an Eph receptor and prevents its signaling (e.g., autophosphorylation). In another specific embodiment, an intrabody binds to the cytoplasmic domain of a B-type Ephrin (e.g., EphrinB1, EphrinB2 or EphrinB3).

PCT Publication WO2011003896 and European Patent Application EP2275442 describe intracellular functional PCNA-Chromobodies made using nucleic acid molecule encoding a polypeptide specifically binding to proliferating cell nuclear antigen (PCNA). Examples of such polypeptides comprising conservative substitutions of one or more amino acids in one or two framework regions are represented by sequence identification numbers 16 and 18 disclosed therein, including the framework region of the polypeptide. In the examples, the framework regions as well as the CDR regions involved in the binding of PCNA have been determined.

European Patent Application EP2703485 describes a method for selecting plasma cells or plasmablasts, as well as for producing target antigen specific antibodies, and novel monoclonal antibodies. In one embodiment, cells expressing intracellular immunoglobulin were identified.

### Proteins and Variants

Glycan-interacting antibodies of the present invention may exist as a whole polypeptide, a plurality of polypeptides or fragments of polypeptides, which independently may be encoded by one or more nucleic acids, a plurality of nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the glycan-interacting antibodies of the invention may comprise naturally occurring amino acids and as such may be considered to be proteins, peptides, polypeptides, or fragments thereof.

Alternatively, the glycan-interacting antibodies may comprise both naturally and non-naturally occurring amino acids.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80%, more preferably at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The present invention contemplates several types of glycan-interacting antibodies which are amino acid based including variants and derivatives. These include substitutional, insertional, deletion and covalent variants and derivatives. As such, included within the scope of this invention are glycan-interacting antibody molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions.

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Isotopic Variations

The glycan-interacting antibodies of the present invention may contain one or more atoms that are isotopes. As used herein, the term “isotope” refers to a chemical element that has one or more additional neutron. In one embodiment, compounds of the present invention may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. The glycan-interacting antibodies may be deuterated in order to change a physical property of the compound, such as stability, or to allow the compounds to be used in diagnostic and experimental applications.

### Conjugates and Combinations

It is contemplated by the present invention that the glycan-interacting antibodies of the present invention may be complexed, conjugated or combined with one or more homologous or heterologous molecules. As used herein, “homologous molecule” means a molecule which is similar in at least one of structure or function relative to a starting molecule while a “heterologous molecule” is one that differs in at least one of structure or function relative to a starting molecule. Structural homologs are therefore molecules which are substantially structurally similar. They can be identical. Functional homologs are molecules which are substantially functionally similar. They can be identical.

Glycan-interacting antibodies of the invention may comprise conjugates. Such conjugates of the invention may include a naturally occurring substance or ligand, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

The conjugates can also include targeting groups, e.g., a cell or tissue targeting agent or group, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

In still other embodiments, glycan-interacting antibodies are covalently conjugated to a cell penetrating polypeptide. The cell-penetrating peptide may also include a signal sequence. The conjugates of the invention can be designed to have increased stability; increased cell transfection; and/or altered biodistribution (e.g., targeted to specific tissues or cell types).

Conjugating moieties may be added to glycan-interacting antibodies such that they allow labeling or flagging targets for clearance. Such tagging/flagging molecules include, but are not limited to ubiquitin, fluorescent molecules, human influenza hemaglutinin (HA), c-myc [a 10 amino acid segment of the human protooncogene myc with sequence EQKLISEEDL (SEQ ID NO: 69)], histidine (His), flag [a short peptide of sequence DYKDDDDK (SEQ ID NO: 70)], glutathione S-transferase (GST), V5 (a paramyxovirus of simian virus 5 epitope), biotin, avidin, streptavidin, horse radish peroxidase (HRP) and digoxigenin.

In some embodiments, glycan-interacting antibodies may be combined with one another or other molecule in the treatment of a disease or condition.

### Nucleic Acids

The present invention embraces nucleic acid molecules. In some embodiments, nucleic acids encode antibodies of the invention (including, but not limited to antibodies, antibody fragments, intrabodies and chimeric receptor antigens). Such nucleic acid molecules include, without limitation, DNA molecules, RNA molecules, polynucleotides, oligonucleotides, mRNA molecules, vectors, plasmids and other constructs. As used herein, the term “construct” refers to any recombinant nucleic acid molecule including, but not limited to plasmids, cosmids, autonomously replicating polynucleotide molecules or linear or circular single-stranded or double-stranded DNA or RNA polynucleotide molecules. The present invention also embraces cells programmed or generated to express nucleic acid molecules encoding glycan-interacting antibodies. Such cells may be generated throughout the use of transfection, electroporation, viral delivery and the like. Viruses engineered with constructs of the invention may include, but are not limited to lentiviruses, adenoviruses, adeno-associated viruses and phages. In some cases, nucleic acids of the invention include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety.

### II. METHODS AND USES

### Therapeutics

### Cancer-Related Applications

Aberrant glycosylation is a hallmark of cancer cell transformation. Multiple aberrant glycosylation forms have been described in human cancers, identifying specific tumor-associated carbohydrate antigens (TACAs) as a class of cell surface molecules suitable for specific tumor targeting (Cheever, M. A. et al., Clin Cancer Res. 2009 Sep. 1; 15(17):5323-37). TACA antigen expression has been found in epithelial cancers including, but not limited to, breast, colon, lung, bladder, cervical, ovarian, stomach, prostate, and liver. TACA antigen expression has been found in embryonal cancers including, but not limited to, yolk sac tumors and seminomas. In addition, TACA antigen expression has been found in many melanomas, carcinomas, and leukemias of various tissues (Heimburg-Molinaro et al., Vaccine. 2011 Nov. 8: 29(48):8802-8826). Antibodies of the present invention that target one or more TACA are referred to herein as “anti-TACA antibodies.”

MUC1 is a key cell surface glycoprotein that is normally extensively glycosylated but is underglycosylated in tumor cells. Sparse glycosylation of MUC1 leads to exposure of immunogenic antigens. These may be along the MUC1 core peptide sequence or along core carbohydrate residues. These TACAs include, but are not limited to N-acetylgalactosamine (Tn), sialyl(α2,6)N-acetylgalactosamine (STn) and galactose (β1-3)N-acetylgalactosamine (also known as Thomsen-Friedenreich antigen or TF). It has been estimated that about 80% of all carcinomas express Tn among the core carbohydrates of MUC1 with STn being strongly expressed on human carcinoma cells and linked to cancer progression and metastasis. With few exceptions, Tn and STn are not expressed in normal healthy tissues. Sialic acid forms a prominent epitope on STn. The invention takes advantage of the fact that aberrant Neu5Gc-STn (GcSTn) glycan expression appears to be highly specific to various carcinomas.

In the case of MUC1, Neu5Gc incorporation into STn yields a tumor-specific target, a site that is an attractive target for antibody-based therapies to treat tumor tissue. In some embodiments of the present invention, glycan-interacting antibodies target MUC1 expressing cancer cells comprising Neu5Gc. To date, Neu5Gc has been detected in glycoconjugates from a number of human cancer tissues including, but not limited to colon cancer, retinoblastoma tissue, melanoma, breast cancer and yolk sac tumor tissue. In some embodiments of the present invention, methods are contemplated for glycan-interacting antibody treatment of these forms of cancer as well as other forms of cancer, not specifically listed here, characterized by the presence of cancer cells comprising Neu5Gc.

Additional antigens comprising glycans have been identified that are expressed in correlation with cancer (Heimburg-Molinaro, J. et al., Cancer vaccines and carbohydrate epitopes. Vaccine. 2011 Nov. 8; 29(48):8802-26). These tumor-associated carbohydrate antigens include, but are not limited to blood group Lewis related antigens [including, but not limited to Lewis^{Y} (Le^{Y}), Lewis^{X} (Le^{X}), Sialyl Lewis^{X} (SLe^{X}) and Sialyl Lewis^{A} (SLe^{A})], glycosphingolipid-related antigens [including, but not limited to Globo H, stage-specific embryonic antigen-3 (SSEA-3) and glycosphingolipids comprising sialic acid], ganglioside-related antigens [including, but not limited to gangliosides GD2, GD3, GM2, fucosyl GM1 and Neu5GcGM3] and polysialic acid-related antigens.

In some embodiments, therapeutics of the present invention may be directed toward Lewis blood group antigens. Lewis blood group antigens comprise a fucose residue linked to GlcNAc by an α1-3 linkage or an α1-4 linkage. They may be found on both glycolipids and glycoproteins. Lewis blood group antigens may be found in the body fluid of individuals that are secretors of these antigens. Their appearance on red cells is due to absorption of Lewis antigens from the serum by the red cells.

In some embodiments, therapeutics of the present invention may be directed toward Le^{Y}. Le^{Y} (also known as CD174) is made up of Galβ1,4GlcNAC comprising α1,2- as well as α1,3-linked fucose residues yielding the Fucα(1,2)Galβ(1,4)Fucα(1,3)GlcNAc epitope. It is synthesized from the H antigen by α1,3 fucosyltransferases which attach the α1,3 fucose to the GlcNAc residue of the parent chain. Le^{Y} may be expressed in a variety of cancers including, but not limited to ovarian, breast, prostate, colon, lung and epithelial. Due to its low expression level in normal tissues and elevated expression level in many cancers, the Le^{Y} antigen is an attractive target for therapeutic antibodies.

In some embodiments, therapeutics of the present invention may be directed toward Le^{X}. Le^{X} comprises the epitope Galβ1-4(Fucα1-3)GlcNAcβ-R. It is also known as CD15 and stage-specific embryonic antigen-1 (SSEA-1). This antigen was first recognized as being immunoreactive with sera taken from a mouse subjected to immunization with F9 teratocarcinoma cells. Le^{X} was also found to correlate with embryonic development at specific stages. It is also expressed in a variety of tissues both in the presence and absence of cancer, but can also be found in breast and ovarian cancers where it is only expressed by cancerous cells.

In some embodiments, therapeutics of the present invention may be directed toward SLe^{A} and/or SLe^{X}. SLe^{A} and SLe^{X} comprise the structures [Neu5Acα2-3Galβ1-3(Fucα1-4)GlcNAcβ-R] and [Neu5Acα2-3Galβ1-4(Fucα1-3)GlcNAcβ-R] respectively. Their expression is upregulated in cancer cells. The presence of these antigens in serum correlates with malignancy and poor prognosis. SLe^{X} is mostly found as a mucin terminal epitope. It is expressed in a number of different cancers including breast, ovarian, melanoma, colon, liver, lung and prostate. In some embodiments of the present invention, SLe^{A} and SLe^{X} targets comprise Neu5Gc (referred to herein as GcSLe^{A} and GcSLe^{X}, respectively).

In some embodiments, therapeutics of the present invention may be directed toward glycolipids and/or epitopes present on glycolipids, including, but not limited to glycosphingolipids. Glycosphingolipids comprise the lipid ceramide linked to a glycan by the ceramide hydroxyl group. On the cell membrane, glycosphingolipids form clusters referred to as “lipid rafts”.

In some embodiments, therapeutics of the present invention may be directed toward Globo H. Globo H is a cancer-related glycosphingolipid first identified in breast cancer cells. The glycan portion of Globo H comprises Fucα(1-2)Galβ(1-3)GalNAcβ(1-3)Galα(1-4)Galβ(1-4)Glcβ(1). Although found in a number of normal epithelial tissues, Globo H has been identified in association with many tumor tissues including, but not limited to, small cell lung, breast, prostate, lung, pancreatic, gastric, ovarian and endometrial tumors.

In some embodiments, therapeutics of the present invention may be directed toward gangliosides. Gangliosides are glycosphingolipids comprising sialic acid. According to ganglioside nomenclature, G is used as an abbreviation for ganglioside. This abbreviation is followed by the letters M, D or T referring to the number of sialic acid residues attached (1, 2 or 3 respectively). Finally the numbers 1, 2 or 3 are used to refer to the order of the distance each migrates when analyzed by thin layer chromatography (wherein 3 travels the greatest distance, followed by 2 and then 1). Gangliosides are known to be involved in cancer-related growth and metastasis and are expressed on the cell surface of tumor cells. Gangliosides expressed on tumor cells include, but are not limited to GD2, GD3, GM2 and fucosyl GM1 (also referred to herein as Fuc-GM1). In some embodiments of the present invention, glycan-interacting antibodies are directed toward GD3. GD3 is a regulator of cell growth. In some embodiments, GD3-directed antibodies are used to modulate cell growth and/or angiogenesis. In some embodiments, GD3-directed antibodies are used to modulate cell attachment. In some embodiments of the present invention, glycan interacting antibodies are directed toward GM2. In some embodiments, GM2-directed antibodies are used to modulate cell to cell contact. In some embodiments, ganglioside targets of the present invention comprise Neu5Gc. In some embodiments, such targets may include a GM3 variant comprising Neu5Gc (referred to herein as GcGM3). The glycan component of GcGM3 is Neu5Gcα2-3Galβ1-4Glc. GcGM3 is a known component of tumor cells.

In some embodiments, TACAs targeted by anti-TACA antibodies of the present invention may include, but are not limited to any of those listed in US Publication Nos. US2013/0236486A1, US2013/0108624A1, US2010/0178292A1, US2010/0104572A1, US2012/0039984A1, US2009/0196916A1, and US2009/0041836A1, the contents of each of which are herein incorporated by reference in their entirety.

### STn in Cancer

The immune system has multiple mechanisms for promoting anti-tumor cell immune activity including both innate and adaptive immune activity. As used herein, the term “anti-tumor cell immune activity” refers to any activity of the immune system that kills or prevents growth and/or proliferation of tumor cells. In some cases, anti-tumor immune activity includes recognition and tumor cell killing by natural killer (NK) cells and phagocytosis by macrophages. Adaptive anti-tumor immune responses include tumor antigen uptake and presentation by antigen presenting cells (APCs,) such as dendritic cells (DCs,) leading to modulation of T cell anti-tumor activity and/or expansion of B cells with secretion of tumor-specific antibodies. The binding of tumor-specific antibodies to tumors can lead to antibody-dependent cellular cytotoxicity (ADCC) and complement-dependent cytotoxicity (CDC) mechanisms of tumor cell death.

As used herein, the term “immune-resistant tumor cell” refers to a tumor cell that reduces or evades anti-tumor cell immune activity. Some studies indicate that the expression of STn (a known TACA) on tumor cell surfaces or secreted into the tumor cell microenvironment can promote tumor cell evasion of anti-tumor immune activity. As used herein, the term “tumor cell microenvironment” refers to any area adjacent to or surrounding a tumor cell. Such areas include, but are not limited to areas between tumor cells, between tumor and non-tumor cells, surrounding fluids and surrounding components of the extracellular matrix.

Sialylated mucins comprising STn were demonstrated by Ogata et al to reduce NK cell targeting of tumor cells (Ogata, S. et al., 1992. Canc. Res. 52:4741-6, the contents of which are herein incorporated by reference in their entirety). This study found that the presence of ovine, bovine and porcine submaxillary mucin (OSM, BSM and PSM, respectively) led to nearly one hundred percent inhibition of cytotoxicity (see Table 2 of Ogata et al). Further studies by Jandus et al, demonstrate that some tumor cells can evade NK destruction due to the expression of sialoglycan ligands that can interact with NK cell siglec receptors, leading to NK inhibition (Jandus, C. et al., 2014, JCI. pii: 65899, the contents of which are herein incorporated by reference in their entirety).

Studies by Toda et al., demonstrate that STn may bind CD22 receptors on B cells, leading to decreased signal transduction and reduced B cell activation (Toda, M. et al., 2008. Biochem Biophys Res Commun. 372(1):45-50, the contents of which are herein incorporated by reference in their entirety). Dendritic cells (DCs) can affect adaptive immune activity by modulating T cell activity. Studies by Carrascal et al found that STn expression by bladder cancer cells induced tolerance in DCs, reducing their ability to induce anti-tumor cell immune activity in T cells (Carrascal, M A et al., 2014. Mol Oncol. pii: S1574-7891(14)₀₀₀₄₇-7, the contents of which are herein incorporated by reference in their entirety). These studies revealed that DCs coming into contact with STn-positive bladder cancer cells displayed a tolorigenic expression profile with low expression of CD80, CD86, IL-12 and TNF-α. Further, DCs were found to modulate regulatory T cells such that the T cells had low expression of IFNγ and high expression of FoxP3. Other studies by van Vliet and others, indicate that DC surface expression of macrophage galactose-type lectin (MGL) can lead to targeting of those cells to tumor tissues (van Vliet, S J., 2007. Amsterdam: Vrije Universiteit. p1-232 and van Vliet, S J. et al., 2008. J Immunol. 181(5):3148-55, Nollau, P. et al., 2013. J Histochem Cytochem. 61(3):199-205, the contents of each of which are herein incorporated by reference in their entirety). DCs arriving at tissues due to MGL interactions may influence T helper (Th) cells in one of three ways. DCs can induce T cell tolerance, T cell immune activity or downregulation of effector T cells. MGL has been shown to bind to both AcSTn and GcSTn and the affinity has been analyzed in depth (Mortezai, N. et al., 2013. Glycobiology. 23(7):844-52, the contents of which are herein incorporated by reference in their entirety). Interestingly, MUC1 expression on tumors has been shown to lead to T cell tolerance, protecting tumor cells from immune eradication.

In some embodiments, glycan-interacting antibodies (including, but not limited to anti-STn antibodies) of the present invention may be used to treat subjects comprising one or more tumor cells expressing one or more TACAs. In some cases, glycan-interacting antibodies (including, but not limited to anti-STn antibodies) of the invention may be used to increase anti-tumor cell immune activity toward tumor cells expressing STn. Such antibodies may increase the adaptive immune response and/or the innate immune response toward immune-resistant tumor cells. Some glycan-interacting antibodies may be used to increase NK anti-tumor cell activity. Such glycan-interacting antibodies may, in some cases, block the interaction between glycan receptors expressed on NK cells and STn glycans on cancer cells or in surrounding tissues.

In some embodiments, glycan-interacting antibodies (including, but not limited to anti-STn antibodies) of the invention may be used to increase B cell anti-tumor cell activity. Such antibodies may reduce the interaction between CD22 receptors on B cells and STn glycans on cancer cells or in surrounding tissues. A study by Sjoberg et al. demonstrates that 9-O-acetylation of α2,6-linked sialic acids on glycoproteins also reduced interaction between B cell CD22 receptors and such glycoproteins (Sjoberg, E. R. et al. 1994. JCB. 126(2): 549-562). Another study by Shi et al. reveals that higher levels of 9-O-acetylated sialic acid residues on murine erythroleukemia cells makes these cells more susceptible to complement-mediated lysis (Shi, W-X. et al., 1996. J of Biol Chem. 271(49): 31526-32, the contents of which are herein incorporated by reference in their entirety). In some embodiments, anti-STn antibodies of the invention are capable of selectively binding non-9-O-acetylated STn, reducing overall STn binding, but reducing tumor cell growth and/or proliferation. (e.g. through increased B cell anti-tumor activity and increased complement-mediated tumor cell destruction). In some embodiments, glycan-interacting antibodies (including, but not limited to anti-STn antibodies) of the invention may be used to increase DC anti-tumor activity. Such antibodies may be used to reduce DC tolerance to tumor cells. Reduced DC tolerance may comprise increasing DC expression of CD80, CD86, IL-12 and/or TNF-α. In some cases, DC anti-tumor cell activity may comprise promotion of T cell anti-tumor cell activity. Such antibodies may prevent binding between DC MGL and glycans expressed on or around cancer cells.

A study by Ibrahim et al. suggests that high levels of anti-STn antibodies along with endocrine therapy may increase overall survival and time to progression (TTP) in women with metastatic breast cancer (Ibrahim, N. K. et al., 2013. 4(7): 577-584, the contents of which are herein incorporated by reference in their entirety). In this study, anti-STn antibody levels were elevated after vaccination with STn linked to keyhole-limpet Hemocyanin (KLH). In some embodiments, anti-STn antibodies of the invention may be used in combination with endocrine therapy (e.g. tamoxifen and/or an aromatase inhibitor).

### Immune-Related Targets

In some embodiments, glycan-interacting antibodies of the invention may be immunomodulatory antibodies. As used herein, an immunomodulatory antibody is an antibody that enhances or suppresses one or more immune function or pathway.

Many bacterial glycans are known to comprise sialic acid. In some cases, such glycans allow bacteria to evade the innate immune system of hosts, including, but not limited to humans. In one example, bacterial glycans inhibit alternate complement pathway activation through factor H recognition. In another example, bacterial glycans mask underlying residues that may be antigenic. Some bacterial glycans participate in cell signaling events through activation of inhibitory sialic acid binding Ig-like lectins (Siglecs) that dampen the immune response to entities comprising certain sialylated moieties (Chen, X. et al., Advances in the biology and chemistry of sialic acids. ACS Chem Biol. 2010 Feb. 19; 5(2):163-76). In some embodiments, glycan-interacting antibodies of the present invention may be used to treat immune complications related to bacterial glycans.

Due to the foreign nature of Neu5Gc as described herein, some Neu5Gc glycans are immunogenic resulting in immune related destruction of cells and other entities where these glycans may be expressed. Such autoimmune destruction may be pathogenic. In some embodiments, glycan-interacting antibodies may be used to treat patients suffering from autoimmune disorders related to Neu5Gc glycans.

In some embodiments, immunomodulatory antibodies of the invention may be used to promote or suppress T cell-mediated immunity. Such antibodies may interact with one or more glycans present on T cells, T cell-related proteins and/or on one or more other cell types that interact with T cells. Immunomodulatory antibodies that enhance T cell mediated immunity may be used to stimulate T cell mediated targeting of cancer cells.

In some tumors, infiltration by tumor-associated macrophages (TAMs) may lead to immunosuppression promoting tumor cell viability and growth. This is thought to be due to immunosuppressive cell signaling that occurs through interactions between myeloid C-type lectin receptors (CLRs) present on TAMs and tumor-associated mucins (Allavena, P. et al., Clin Dev Immunol. 2010; 2010:547179). In some embodiments, binding of immunomodulatory antibodies of the invention to one or more tumor-associated mucin or TACA prevents immunosuppressive cell signaling in TAMs.

### Anti-Viral Applications

In some embodiments, glycan-interacting antibodies of the invention may target viruses. Viral coat proteins and viral envelopes often comprise glycans, referred to herein as viral surface glycans. Such glycans may be targets of glycan-interacting antibodies. In some embodiments, viral surface glycans comprise sialyl-STn. In a further embodiment, viral surface glycans comprise GcSTn. Viruses that may be targeted by glycan-interacting antibodies include, but are not limited to HIV, influenza, rhinovirus, varicella-zoster, rotavirus, herpes (e.g. types 1 and 2), hepatitis (e.g. types A, B, C, D and E), yellow fever and human papillomavirus.

### Other Therapeutic Applications

In some embodiments, glycan-interacting antibodies of the invention may act to alter or control proteolytic events. In some embodiments, glycan-interacting antibodies of the present invention may be internalized into cells prior to binding to targets.

### Veterinary Applications

It is contemplated that glycan-interacting antibodies of the invention will find utility in the area of veterinary care including the care and treatment of non-human vertebrates. As described herein, the term “non-human vertebrate” includes all vertebrates with the exception of Homo sapiens, including wild and domesticated species such as companion animals and livestock. Non-human vertebrates include mammals, such as alpaca, banteng, bison, camel, cat, cattle, deer, dog, donkey, gayal, goat, guinea pig, horse, llama, mule, pig, rabbit, reindeer, sheep water buffalo, and yak. Livestock includes domesticated animals raised in an agricultural setting to produce materials such as food, labor, and derived products such as fiber and chemicals. Generally, livestock includes all mammals, avians and fish having potential agricultural significance. In particular, four-legged slaughter animals include steers, heifers, cows, calves, bulls, cattle, swine and sheep.

### Bioprocessing

In some embodiments of the invention are methods for producing biological products in host cells by contacting the cells with one or more glycan-interacting antibody (such as an antibody or fusion protein) capable of modulating gene expression, or altering levels and/or types of glycans produced wherein such modulation or alteration enhances production of biological products. According to the present invention, bioprocessing methods may be improved by using one or more of the glycan-interacting antibodies of the present invention. They may also be improved by supplementing, replacing or adding one or more glycan-interacting antibodies.

### III. PHARMACEUTICAL COMPOSITIONS

The pharmaceutical compositions described herein can be characterized by one or more of bioavailability, therapeutic window and/or volume of distribution.

### Bioavailability

Glycan-interacting antibodies, when formulated into a composition with a delivery/formulation agent or vehicle as described herein, can exhibit an increase in bioavailability as compared to a composition lacking a delivery agent as described herein. As used herein, the term “bioavailability” refers to the systemic availability of a given amount of glycan-interacting antibodies administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of a glycan-interacting antibody, measured as AUC, Cₘₐₓ, or Cₘᵢₙ in a mammal is greater, when co-administered with a delivery agent as described herein, than when such co-administration does not take place. In some embodiments, the bioavailability of the glycan-interacting antibody can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

Glycan-interacting antibodies, when formulated into a composition with a delivery agent as described herein, can exhibit an increase in the therapeutic window of the administered glycan-interacting antibody composition as compared to the therapeutic window of the administered glycan-interacting antibody composition lacking a delivery agent as described herein. As used herein “therapeutic window” refers to the range of plasma concentrations, or the range of levels of therapeutically active substance at the site of action, with a high probability of eliciting a therapeutic effect. In some embodiments, the therapeutic window of the glycan-interacting antibody when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

Volume of distribution Glycan-interacting antibodies, when formulated into a composition with a delivery agent as described herein, can exhibit an improved volume of distribution (V_{dist}), e.g., reduced or targeted, relative to a composition lacking a delivery agent as described herein. The volume of distribution (V_{dist}) relates the amount of the drug in the body to the concentration of the drug in the blood or plasma. As used herein, the term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} can be used to determine a loading dose to achieve a steady state concentration. In some embodiments, the volume of distribution of the glycan-interacting antibody when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

In some embodiments, glycan-interacting antibodies comprise compositions and/or complexes in combination with one or more pharmaceutically acceptable excipients. Pharmaceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, compositions are administered to humans, human patients or subjects. For the purposes of the present disclosure, the phrase “active ingredient” generally refers to glycan-interacting antibodies to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient. In one embodiment, active ingredients are antibodies directed toward glycans.

### Formulations

Glycan-interacting antibodies of the invention can be formulated using one or more excipients to: (1) increase stability; (2) increase cell permeability; (3) permit the sustained or delayed release (e.g., from a formulation of the glycan-interacting antibody); and/or (4) alter the biodistribution (e.g., target the glycan-interacting antibody to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, formulations of the present invention can include, without limitation, liposomes, lipid nanoparticles, polymers, lipoplexes, core-shell nanoparticles, peptides, proteins, cells transfected with the glycan-interacting antibodies (e.g., for transplantation into a subject) and combinations thereof.

### Excipients

As used herein, the term “excipient” refers to any substance combined with a compound and/or composition of the invention before use. In some embodiments, excipients are inactive and used primarily as a carrier, diluent or vehicle for a compound and/or composition of the present invention. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, M D, 2006; incorporated herein by reference).

The use of a conventional excipient medium is contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [Span®60], sorbitan tristearate [Span®65], glyceryl monooleate, sorbitan monooleate [SPAN®80]), polyoxyethylene esters (e.g. polyoxyethylene monostearate [MYRJ©45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN® II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Vehicles

### Liposomes, Lipoplexes and Lipid Nanoparticles

Glycan-interacting antibodies of the present invention may be formulated using one or more liposomes, lipoplexes, or lipid nanoparticles. In one embodiment, pharmaceutical compositions comprising glycan-interacting antibodies further comprise liposomes. Liposomes are artificially-prepared vesicles which may primarily comprise one or more lipid bilayers and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment such formulations may also be constructed or compositions altered such that they passively or actively are directed to different cell types in vivo.

Formulations can also be selectively targeted through expression of different ligands on their surface as exemplified by, but not limited by, folate, transferrin, N-acetylgalactosamine (GalNAc), and antibody targeted approaches.

Liposomes, lipoplexes, or lipid nanoparticles may be used to improve the efficacy of glycan-interacting antibody function as these formulations may be able to increase cell transfection with glycan-interacting antibodies. The liposomes, lipoplexes, or lipid nanoparticles may also be used to increase the stability of glycan-interacting antibodies.

Liposomes that are specifically formulated for antibody cargo are prepared according to techniques known in the art, such as described by Eppstein et al. (Eppstein, D. A. et al., Biological activity of liposome-encapsulated murine interferon gamma is mediated by a cell membrane receptor. Proc Natl Acad Sci USA. 1985 June; 82(11):3688-92); Hwang et al. (Hwang, K. J. et al., Hepatic uptake and degradation of unilamellar sphingomyelin/cholesterol liposomes: a kinetic study. Proc Natl Acad Sci USA. 1980 July; 77(7):4030-4); U.S. Pat. Nos. 4,485,045 and 4,544,545. Production of liposomes with sustained circulation time is also described in U.S. Pat. No. 5,013,556.

Liposomes comprising glycan-interacting antibodies of the present invention may be generated using reverse phase evaporation utilizing lipids such as phosphatidylcholine, cholesterol as well as phosphatidylethanolamine that has been polyethylene glycol-derivatized. Filters with defined pore size are used to extrude liposomes of the desired diameter. In another embodiment, glycan-interacting antibodies of the present invention can be conjugated to the external surface of liposomes by disulfide interchange reaction as is described by Martin et al. (Martin, F. J. et al., Irreversible coupling of immunoglobulin fragments to preformed vesicles. An improved method for liposome targeting. J Biol Chem. 1982 Jan. 10; 257(1):286-8).

### Polymers and Nanoparticles

Glycan-interacting antibodies of the invention can be formulated using natural and/or synthetic polymers. Non-limiting examples of polymers which may be used for delivery include, but are not limited to DMRI/DOPE, poloxamer, chitosan, cyclodextrin, and poly(lactic-co-glycolic acid) (PLGA) polymers. These may be biodegradable.

The polymer formulation can permit the sustained or delayed release of glycan-interacting antibodies (e.g., following intramuscular or subcutaneous injection). The altered release profile for glycan-interacting antibodies can result in, for example, release of the glycan-interacting antibodies over an extended period of time. The polymer formulation may also be used to increase the stability of glycan-interacting antibodies.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; herein incorporated by reference in its entirety).

Glycan-interacting antibodies of the invention can also be formulated as nanoparticles using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so delivery of glycan-interacting antibodies may be enhanced. For glycan-interacting antibodies, systems based on poly(2-(methacryloyloxy)ethyl phosphorylcholine)-block-(2-(diisopropylamino)ethyl methacrylate), (PMPC-PDPA), a pH sensitive diblock copolymer that self-assembles to form nanometer-sized vesicles, also known as polymersomes, at physiological pH may be used. These polymersomes have been shown to successfully deliver relatively high antibody payloads within live cells. (Massignani, et al, Cellular delivery of antibodies: effective targeted subcellular imaging and new therapeutic tool. Nature Proceedings, May, 2010).

In one embodiment, a PEG-charge-conversional polymer (Pitella et al., Biomaterials. 2011 32:3106-3114) may be used to form a nanoparticle to deliver glycan-interacting antibodies of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle.

In one embodiment, matrices of poly(ethylene-co-vinyl acetate), are used to deliver glycan-interacting antibodies of the invention. Such matrices are described in Nature Biotechnology 10, 1446-1449 (1992).

### Antibody Formulations

Glycan-interacting antibodies of the invention may be formulated for intravenous administration or extravascular administration (Daugherty, et al., Formulation and delivery issues for monoclonal antibody therapeutics. Adv Drug Deliv Rev. 2006 Aug. 7; 58(5-6):686-706, US patent publication number 2011/0135570, all of which are incorporated herein in their entirety). Extravascular administration routes may include, but are not limited to subcutaneous administration, intraperitoneal administration, intracerebral administration, intraocular administration, intralesional administration, topical administration and intramuscular administration.

Antibody structures may be modified to improve their effectiveness as therapeutics. Improvements may include, but are not limited to improved thermodynamic stability, reduced Fc receptor binding properties and improved folding efficiency. Modifications may include, but are not limited to amino acid substitutions, glycosylation, palmitoylation and protein conjugation.

Glycan-interacting antibodies may be formulated with antioxidants to reduce antibody oxidation. glycan-interacting antibodies may also be formulated with additives to reduce protein aggregation. Such additives may include, but are not limited to albumin, amino acids, sugars, urea, guanidinium chloride, polyalchohols, polymers (such as polyethylene glycol and dextrans), surfactants (including, but not limited to polysorbate 20 and polysorbate 80) or even other antibodies.

Glycan-interacting antibodies of the present invention may be formulated to reduce the impact of water on antibody structure and function. Antibody preparations in such formulations may be may be lyophilized. Formulations subject to lyophilization may include carbohydrates or polyol compounds to protect and stabilize antibody structure. Such compounds include, but are not limited to sucrose, trehalose and mannitol.

Glycan-interacting antibodies of the present invention may be formulated with polymers. In one embodiment, polymer formulations may contain hydrophobic polymers. Such polymers may be microspheres formulated with polylactide-co-glycolide through a solid-in-oil-in-water encapsulation method. Microspheres comprising ethylene-vinyl acetate copolymer are also contemplated for antibody delivery and may be used to extend the time course of antibody release at the site of delivery. In another embodiment, polymers may be aqueous gels. Such gels may, for example, comprise carboxymethylcellulose. Aqueous gels may also comprise hyaluronic acid hydrogel. Antibodies may be covalently linked to such gels through a hydrazone linkage that allows for sustained delivery in tissues, including but not limited to the tissues of the central nervous system.

### Peptide and Protein Formulations

Glycan-interacting antibodies of the invention may be formulated with peptides and/or proteins. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton FL, 2002); El-Andaloussi et al., Curr. Pharm. Des. 11(28):3597-611 (2003); and Deshayes et al., Cell. Mol. Life Sci. 62(16):1839-49 (2005), all of which are incorporated herein by reference). The compositions can also be formulated to include a cell penetrating agent, e.g., liposomes, which enhance delivery of the compositions to the intracellular space. Glycan-interacting antibodies of the invention may be complexed to peptides and/or proteins such as, but not limited to, peptides and/or proteins from Aileron Therapeutics (Cambridge, MA) and Permeon Biologics (Cambridge, MA) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in their entirety).

In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where glycan-interacting antibodies may be introduced.

In formulations of the present invention, peptides or proteins may be incorporated to increase cell transfection by glycan-interacting antibodies or alter the biodistribution of glycan-interacting antibodies (e.g., by targeting specific tissues or cell types).

### Cell Formulations

Cell-based formulations of glycan-interacting antibody compositions of the invention may be used to ensure cell transfection (e.g., in the cellular carrier) or alter the biodistribution of the compositions (e.g., by targeting the cell carrier to specific tissues or cell types).

### Cell Transfer Methods

A variety of methods are known in the art and are suitable for introduction of nucleic acids or proteins, such as glycan-interacting antibodies, into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are used to deliver nucleic acids in vivo (Yoon and Park, Expert Opin Drug Deliv. 2010 7:321-330; Postema and Gilja, Curr Pharm Biotechnol. 2007 8:355-361; Newman and Bettinger, Gene Ther. 2007 14:465-475; all herein incorporated by reference in their entirety). Sonoporation methods are known in the art and are also taught for example as it relates to bacteria in US Patent Publication 20100196983 and as it relates to other cell types in, for example, US Patent Publication 20100009424, each of which are incorporated herein by reference in their entirety.

Electroporation techniques are also well known in the art and are used to deliver nucleic acids in vivo and clinically (Andre et al., Curr Gene Ther. 2010 10:267-280; Chiarella et al., Curr Gene Ther. 2010 10:281-286; Hojman, Curr Gene Ther. 2010 10:128-138; all herein incorporated by reference in their entirety). In one embodiment, glycan-interacting antibodies may be delivered by electroporation.

### Administration and Delivery

The compositions of the present invention may be administered by any of the standard methods or routes known in the art.

Glycan-interacting antibodies of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Non-limiting routes of administration for glycan-interacting antibodies of the present invention are described below.

### Parenteral and Injectable Administration

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compositions containing glycan-interacting antibodies of the invention may be formulated for administration topically. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Three routes are commonly considered to deliver glycan-interacting antibodies to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). glycan-interacting antibodies can be delivered to the skin by several different approaches known in the art.

In one embodiment, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions and/or glycan-interacting antibodies described herein to allow a user to perform multiple treatments of a subject(s).

In one embodiment, the invention provides for compositions comprising glycan-interacting antibodies to be delivered in more than one injection.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, compositions of the present invention are formulated in depots for extended release. Generally, a specific organ or tissue (a “target tissue”) is targeted for administration.

In some aspects of the invention, glycan-interacting antibodies are spatially retained within or proximal to a target tissue. Provided are methods of providing compositions to one or more target tissue of a mammalian subject by contacting the one or more target tissue (comprising one or more target cells) with compositions under conditions such that the compositions, in particular glycan-interacting antibody component(s) of the compositions, are substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the level of glycan-interacting antibodies present in the compositions entering the target tissues and/or cells. For example, at least 1, 5, 10, 20, 30, 40, 50, 60, 70, 80, 85, 90, 95, 96, 97, 98, 99, 99.9, 99.99 or greater than 99.99% of glycan-interacting antibodies administered to the subject are present intracellularly at a period of time following administration. For example, intramuscular injection to a mammalian subject is performed using an aqueous composition comprising one or more glycan-interacting antibody and a transfection reagent, and retention of the composition is determined by measuring the level of glycan-interacting antibodies present in the muscle cells.

Certain aspects of the invention are directed to methods of providing compositions to target tissues of mammalian subjects, by contacting the target tissues (containing one or more target cells) with compositions under conditions such that the compositions are substantially retained in the target tissue. Compositions contain an effective amount of glycan-interacting antibodies such that the effect of interest is produced in at least one target cell. Compositions generally contain cell penetration agents and a pharmaceutically acceptable carrier, although “naked” glycan-interacting antibodies (such as glycan-interacting antibodies without cell penetration agents or other agents) are also contemplated.

In some embodiments, compositions include a plurality of different glycan-interacting antibodies, where one or more than one of the glycan-interacting antibodies targets a glycan of interest. Optionally, compositions also contain cell penetration agents to assist in the intracellular delivery of compositions. A determination is made of the composition dose required to target glycans of interest in a substantial percentage of cells contained within a predetermined volume of the target tissue (generally, without targeting glycans in tissue adjacent to the predetermined volume, or distally to target tissues). Subsequent to this determination, the determined dose may be introduced directly into the tissue of the mammalian subject.

In one embodiment, the invention provides for glycan-interacting antibodies to be delivered in more than one injection or by split dose injections.

### Pulmonary Administration

Pharmaceutical compositions may be prepared, packaged, and/or sold in formulations suitable for pulmonary administration via the buccal cavity. Such formulations may comprise dry particles further comprising active ingredients and having a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.10% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic or Otic Administration

A pharmaceutical composition may be prepared, packaged, and/or sold in a formulation suitable for ophthalmic or otic administration. Such formulations may, for example, be in the form of eye or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Subretinal inserts may also be used as a form of administration.

### Payload Administration

Glycan-interacting antibodies described herein may be used in a number of different scenarios in which delivery of a substance (the “payload”) to a biological target is desired, for example delivery of detectable substances for detection of the target, or delivery of a therapeutic or diagnostic agent. Detection methods can include, but are not limited to, both imaging in vitro and in vivo imaging methods, e.g., immunohistochemistry, bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

Glycan-interacting antibodies can be designed to include both a linker and a payload in any useful orientation. For example, a linker having two ends is used to attach one end to the payload and the other end to the glycan-interacting antibody. The glycan-interacting antibodies of the invention can include more than one payload as well as a cleavable linker. In another example, a drug that may be attached to glycan-interacting antibodies via a linker and may be fluorescently labeled can be used to track the drug in vivo, e.g. intracellularly.

Other examples include, but are not limited to, the use of glycan-interacting antibodies in reversible drug delivery into cells.

Glycan-interacting antibodies described herein can be used in intracellular targeting of a payload, e.g., detectable or therapeutic agents, to specific organelles. In addition, glycan-interacting antibodies described herein may be used to deliver therapeutic agents to cells or tissues, e.g., in living animals. For example, glycan-interacting antibodies described herein may be used to deliver chemotherapeutic agents to kill cancer cells. glycan-interacting antibodies attached to therapeutic agents through linkers can facilitate member permeation allowing the therapeutic agent to travel into a cell to reach an intracellular target.

In some embodiments, the payload may be a therapeutic agent such as a cytotoxin, radioactive ion, chemotherapeutic, or other therapeutic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). In the case of anti-STn antibodies of the present invention, tumor killing may be boosted by the conjugation of a toxin to such anti-STn antibodies.

In some embodiments, the payload may be a detectable agent, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine (1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodanine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Combinations

Glycan-interacting antibodies may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, and/or imaging compositions in combination with agents that may improve their bioavailability, reduce and/or modify their metabolism, inhibit their excretion, and/or modify their distribution within the body.

### Dosage

The present disclosure encompasses delivery of glycan-interacting antibodies for any of therapeutic, pharmaceutical, diagnostic or imaging by any appropriate route taking into consideration likely advances in the sciences of drug delivery. Delivery may be naked or formulated.

### Naked Delivery

Glycan-interacting antibodies of the present invention may be delivered to cells, tissues, organs or organisms in naked form. As used herein in, the term “naked” refers to glycan-interacting antibodies delivered free from agents or modifications which promote transfection or permeability. Naked glycan-interacting antibodies may be delivered to cells, tissues, organs and/or organisms using routes of administration known in the art and described herein. Naked delivery may include formulation in a simple buffer such as saline or PBS.

### Formulated Delivery

Glycan-interacting antibodies of the present invention may be formulated, using methods described herein. Formulations may comprise glycan-interacting antibodies which may be modified and/or unmodified. Formulations may further include, but are not limited to, cell penetration agents, pharmaceutically acceptable carriers, delivery agents, bioerodible or biocompatible polymers, solvents, and sustained-release delivery depots. Formulated glycan-interacting antibodies may be delivered to cells using routes of administration known in the art and described herein.

Compositions may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Dosing

The present invention provides methods comprising administering one or more glycan-interacting antibodies in accordance with the invention to a subject in need thereof. Nucleic acids encoding glycan-interacting antibodies, proteins or complexes comprising glycan-interacting antibodies, or pharmaceutical, imaging, diagnostic, or prophylactic compositions thereof, may be administered to a subject using any amount and any route of administration effective for preventing, treating, diagnosing, or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

According to the present invention, glycan-interacting antibodies may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in a 24 hr period. It may be administered as a single unit dose. In one embodiment, glycan-interacting antibodies of the present invention are administered to a subject in split doses. Glycan-interacting antibodies may be formulated in buffer only or in a formulation described herein. Pharmaceutical compositions comprising glycan-interacting antibodies as described herein may be formulated into a dosage form described herein, such as a topical, intranasal, intratracheal, or injectable (e.g., intravenous, intraocular, intravitreal, intramuscular, intracardiac, intraperitoneal or subcutaneous). General considerations in the formulation and/or manufacture of pharmaceutical agents may be found, for example, in Remington: The Science and Practice of Pharmacy 21st ed., Lippincott Williams & Wilkins, 2005 (incorporated herein by reference).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### IV. KITS AND DEVICES

### Kits

Any of the compositions described herein may be comprised in a kit. In a non-limiting example, reagents for generating glycan-interacting antibodies, including antigen molecules are included in a kit. The kit may further include reagents or instructions for creating or synthesizing glycan-interacting antibodies. It may also include one or more buffers. Other kits of the invention may include components for making glycan-interacting antibody protein or nucleic acid arrays or libraries and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the glycan-interacting antibodies, e.g., proteins, nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried powder. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least 1000 micrograms or at most 10 g of dried dye are provided in kits of the invention. The dye may then be resuspended in any suitable solvent, such as DMSO.

A kit may include instructions for employing the kit components as well the use of any other reagent not included in the kit. Instructions may include variations that can be implemented.

### Devices

Any of the compositions described herein may be combined with, coated onto or embedded in a device. Devices include, but are not limited to, dental implants, stents, bone replacements, artificial joints, valves, pacemakers or other implantable therapeutic devices.

### V. EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc). can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Immunization Using Alternative Adjuvants, Antigens and Mouse Strains

An immunization study was carried out to develop mice with immune responses to sialylated antigens using enhanced adjuvants. 40 each of Cmah−/− (male and female, ˜6-8 weeks old) and C57BL/6 mice (females, 6-8 weeks old) were acclimated for at least 3 days and given access to standard diet (2920X.10, Global 18% Protein Rodent Diet, Harlan, San Diego, CA) and acidified water (pH 2.7-3.0) ad libitum throughout the study period. Mice from each strain (Cmah−/− and C57BL/6) were divided into 4 groups of 10 mice each (a total of 8 groups).

Mice were immunized according to the study design shown in the following Table using either PSM or OSM at doses of either 10 μg or 100 μg (from 1 mg/ml stock solution) depending on the adjuvant used. Adjuvants included either Freund's adjuvant (complete or incomplete) or enhanced adjuvants comprising AbiSCO-100 (12 μg) and ODN-2395 (100 μg). Mice were vaccinated on days 0, 14, 28, 42 and 56 of the study and blood was collected for antibody analysis prior to each vaccination. Mice receiving vaccinations with Freund's adjuvant received complete Freund's adjuvant (CFA) with their first vaccination and incomplete Freund's adjuvant (IFA) during subsequent vaccinations.

TABLE 8
Study Design

| Group | Strain | Immunogen and Adjuvant |
|---|---|---|
| 1 | Cmah -/- | PSM (100 μg) + CFA or IFA (100 μl) |
| 2 | Cmah -/- | PSM (10 μg) + AbiSCO-100 (12 μg) + ODN-2395 (100 μg) |
| 3 | Cmah -/- | OSM (100 μg) + CFA or IFA (100 μl) |
| 4 | Cmah -/- | OSM (10 μg) + AbiSCO-100 (12 μg) + ODN-2395 (100 μg) |
| 5 | C57BL/6 | PSM (100 μg) + CFA or IFA (100 μl) |
| 6 | C57BL/6 | PSM (10 μg) + AbiSCO-100 (12 μg) + ODN-2395 (100 μg) |
| 7 | C57BL/6 | OSM (100 μg) + CFA or IFA (100 μl) |
| 8 | C57BL/6 | OSM (10 μg) + AbiSCO-100 (12 μg) + ODN-2395 (100 μg) |

Mice were randomized for placement into individual treatment groups based on body weight and sex. Vaccinations were given by subcutaneous injections around armpits and inguinal regions (50 μl per site, 4 sites for a total of 200 μl per mouse). Additionally, body weight and health observations for each mouse were determined twice per week.

During each blood collection, approximately 0.2 ml of whole blood was collected by facial vein bleed and placed into serum separator tubes. Tubes were then kept at room temperature for at least 30 minutes to allow clotting. Serum was then divided into aliquots and stored at −80° C. until analysis. An additional blood collection was also carried out on day 66 of the study. Blood samples were processed to serum and kept on ice for analysis on the same day.

To determine the titer of anti-STn antibodies, mouse sera collected at day 42 was analyzed by EIA. Plates were coated with coating buffer (50 mM Na carbonate/bicarbonate, pH 9.5, Sigma-Aldrich, St. Louis, MO) containing 1 μg BSM/100 μl overnight at 4° C. The next day, plates were incubated with 0.1 M NaOH for 30 min at 37° C. before being washed with phosphate buffered saline (PBS, pH 7.3, Sigma-Aldrich, St. Louis, MO). Half of the wells in each plate were next treated with either PBS (pH 6.5) or periodate solution [2 mM NaIO₄ (MW=213.98 g/mol) in PBS, pH6.5; Sigma-Aldrich, St. Louis, MO] for 20 min in the dark with gentle shaking. Solutions were removed by washing with PBS (pH 7.4) and then incubated overnight at 4° C. in blocking solution (PBS with 0.1% powdered egg white).

Test samples as well as positive [comprising anti-STn antibody (from mouse hybridoma clone 3F1) from SBH Biosciences, Natick, MA] and negative control samples were prepared by generating serial dilutions in blocking buffer. Blocking solution was removed from blocked plates and sample dilutions were added to wells at a volume of 100 μl/well. Plates were then incubated for 2 hours at room temperature. After washing with PBS with 0.05% Tween-20, wells were treated with goat anti-mouse IgG-HRP (Jackson Immunoresearch Laboratories, Inc., West Grove, PA; 100 μl/well at a dilution of 1:5,000 in PBS). After a one hour incubation at room temperature, wells were washed with PBS with 0.05% Tween-20. To visualize bound secondary antibodies, wells were finally treated with 100 μl/well of HRP substrate. Reactions were stopped with 100 μl/well of 1.6 M sulfuric acid and optical density (OD) values for each well were obtained spectrophotometrically at 490 nm. The highest dilution of each sample tested to result in detectable levels of reaction product (adjusted mean optical density of 0.050 or greater) are listed in the following Table.

TABLE 9
Highest sample dilutions with detectable antibody

| | | Highest sample dilution with | |
|---|---|---|---|
| | Animal | detectable antibody | |
| Group | ID | Day0 | Day42 |
| 1 | #3094 | <1:100 | 1:2500 |
| 1 | #3095 | <1:100 | <1:100 |
| 1 | #3071 | <1:100 | 1:12500 |
| 1 | #3081 | <1:100 | 1:12500 |
| 1 | #3295 | <1:100 | 1:500 |
| 1 | #3099 | <1:100 | 1:2500 |
| 1 | #3083 | <1:100 | 1:12500 |
| 1 | #2793 | <1:100 | 1:100 |
| 1 | #2795 | <1:100 | 1:500 |
| 1 | #3087 | <1:100 | 1:12500 |
| 2 | #3091 | <1:100 | 1:12500 |
| 2 | #3092 | <1:100 | <1:100 |
| 2 | #3074 | <1:100 | 1:12500 |
| 2 | #3096 | <1:100 | 1:12500 |
| 2 | #2791 | <1:100 | 1:2500 |
| 2 | #2792 | <1:100 | 1:12500 |
| 2 | #3097 | <1:100 | <1:100 |
| 2 | #3088 | <1:100 | 1:62500 |
| 2 | #3298 | <1:100 | 1:500 |
| 2 | #2798 | <1:100 | 1:2500 |
| 3 | #3790 | <1:100 | 1:500 |
| 3 | #3090 | <1:100 | 1:12500 |
| 3 | #3084 | <1:100 | 1:2500 |
| 3 | #3082 | <1:100 | 1:500 |
| 3 | #3075 | <1:100 | 1:100 |
| 3 | #3297 | <1:100 | 1:500 |
| 3 | #3793 | <1:100 | 1:2500 |
| 3 | #3085 | <1:100 | 1:2500 |
| 3 | #3098 | <1:100 | 1:500 |
| 3 | #3089 | <1:100 | 1:500 |
| 4 | #3093 | <1:100 | 1:12500 |
| 4 | #3076 | <1:100 | 1:12500 |
| 4 | #3072 | <1:100 | 1:2500 |
| 4 | #3073 | <1:100 | 1:2500 |
| 4 | #3299 | <1:100 | 1:12500 |
| 4 | #3296 | <1:100 | 1:12500 |
| 4 | #3791 | <1:100 | 1:2500 |
| 4 | #2794 | <1:100 | 1:12500 |
| 4 | #3792 | <1:100 | 1:2500 |
| 4 | #2796 | <1:100 | 1:2500 |
| 5 | #4416 | <1:100 | 1:2500 |
| 5 | #4435 | <1:100 | 1:62500 |
| 5 | #4420 | <1:100 | 1:2500 |
| 5 | #4402 | <1:100 | 1:2500 |
| 5 | #4415 | <1:100 | <1:100 |
| 5 | #4439 | <1:100 | 1:100 |
| 5 | #4405 | <1:100 | <1:100 |
| 5 | #4433 | <1:100 | 1:100 |
| 5 | #4412 | <1:100 | 1:500 |
| 5 | #4426 | <1:100 | <1:100 |
| 6 | #4427 | <1:100 | 1:62500 |
| 6 | #4434 | <1:100 | 1:2500 |
| 6 | #4423 | <1:100 | 1:12500 |
| 6 | #4418 | <1:100 | 1:12500 |
| 6 | #4436 | <1:100 | 1:62500 |
| 6 | #4438 | <1:100 | 1:12500 |
| 6 | #4432 | <1:100 | <1:100 |
| 6 | #4421 | <1:100 | 1:2500 |
| 6 | #4428 | <1:100 | 1:2500 |
| 6 | #4401 | <1:100 | 1:12500 |
| 7 | #4419 | <1:100 | 1:2500 |
| 7 | #4413 | <1:100 | 1:100 |
| 7 | #4424 | <1:100 | 1:12500 |
| 7 | #4408 | <1:100 | 1:2500 |
| 7 | #4409 | <1:100 | 1:500 |
| 7 | #4417 | <1:100 | 1:2500 |
| 7 | #4437 | <1:100 | 1:100 |
| 7 | #4430 | <1:100 | 1:100 |
| 7 | #4425 | <1:100 | <1:100 |
| 7 | #4429 | <1:100 | <1:100 |
| 8 | #4407 | <1:100 | 1:62500 |
| 8 | #4406 | <1:100 | 1:12500 |
| 8 | #4440 | <1:100 | 1:12500 |
| 8 | #4403 | <1:100 | 1:12500 |
| 8 | #4411 | <1:100 | 1:62500 |
| 8 | #4414 | <1:100 | 1:62500 |
| 8 | #4431 | <1:100 | 1:62500 |
| 8 | #4422 | <1:100 | 1:12500 |
| 8 | #4404 | <1:100 | 1:62500 |
| 8 | #4410 | <1:100 | 1:62500 |

At day 42, the results indicated that group 8 mice, wild type mice immunized with OSM using AbISCO-100 and ODN-2395 adjuvants yielded the most number of animals with high antibody titers. Similar results were obtained when serum harvested at day 66 was tested. Interestingly; however, more deaths occurred in groups immunized using AbISCO-100 and ODN-2395 adjuvants, indicating some toxicity at the doses used (see the following Table).

TABLE 10
Comparison of immunizations at day 42 and 66

| | Day 42 | | Day 66 | |
|---|---|---|---|---|
| | # of mice with | | # of mice with | |
| | detectable levels | | detectable levels | |
| | of antibody in | # of | of antibody in | # of |
| | samples diluted | dead | samples diluted | dead |
| Group | 1:12,500 or greater | mice | 1:12,500 or greater | mice |
| 1 | 4 | 0 | 3 | 1 |
| 2 | 4 | 0 | 6 | 2 |
| 3 | 1 | 0 | 0 | 0 |
| 4 | 5 | 0 | 8 | 1 |
| 5 | 1 | 0 | 2 | 0 |
| 6 | 5 | 0 | 5 | 1 |
| 7 | 1 | 0 | 1 | 0 |
| 8 | 10 | 0 | 7 | 3 |

On day 78 of the study, mice numbers 3074, 3096, 4402, 4418, 4421, 3296 and 4414 were subjected to an additional immunization of antigen with AbISCO-100. Of these mice, numbers 3296 and 4414 received OSM antigen (10 μg/mouse), while the others received PSM as antigen (10 μg/mouse). On day 92 of the study, these mice were bled and subjected to another immunization comprising antigen only. On day 85 of the study, mouse number 4406 was immunized with OSM antigen (100 μg, no adjuvant) and processed for hybridoma formation on day 88.

### Example 2. Anti-STn Animal Serum Titer Determination and Mouse Selection

Anti-STn serum titer is determined using a murine anti-STn bovine submaxillary mucin (BSM) ELISA together with serum profiles observed by glycan microarray. 96-well plates are coated with 1 μg/well of BSM and incubated overnight at 4° C. O-acetylation of BSM antigen is removed by treating wells with 0.1 M sodium hydroxide. Specific binding to STn is determined by treatment of wells with sodium periodate. Periodate treatment destroys the C6 side chain of sialic acid; therefore antibodies raised against STn should not bind to periodate-treated wells. Wells are blocked with PBS 1% ovalbumin (OVA). Serum samples to be assayed are serially diluted in PBS 1% OVA. A commercially available mouse anti-STn monoclonal antibody, 3F1 (SBH Sciences, Natick, MA) is used as a positive control. This antibody is also serially diluted in PBS with 1% OVA. A pool of serum from naïve wild type mice is used for the preparation of negative control samples. Detection of anti-STn antibodies present in serum is determined using an HRP-conjugated polyclonal goat anti-mouse IgG antibody (Jackson Immunoresearch, West Grove, PA). The reaction is stopped by addition of sulfuric acid (1.6 M). Optical densities are measured at 490 nm using a Spectramax microplate reader (Molecular Devices, Sunnyvale, CA). The serum titer is obtained by comparison of OD values with a cutoff value calculated as two standard deviations above the mean of optical density values of the negative control. Sample tests are considered positive if the mean optical density value is greater than the cutoff value.

### Example 3. Comparison of Body Weights, Antibody Titers and Antibody Specificity Between Adjuvants and Antigens Used

Overall results from mouse immunization were compared to provide insight with regard to the success of PSM and OSM antigens to produce high titer responses to immunization as well as to evaluate the specificity of resulting antibodies produced. In the study, mice immunized with OSM were capable of developing a high titer anti-STn response with 17 out of 40 mice developing serum antibody levels detectable in the 1:12,500 dilution sample (such mice are referred to herein as “responders.”) About half of the responders produced antibodies targeting AcSTn specifically and about half produced antibodies targeting pan-STn. Alternatively, there were 18 responders out of 40 mice immunized with PSM with nearly a third of the responders producing anti-STn antibodies targeting GcSTn specifically, over half with antibodies targeting pan-STn and a couple produced anti-STn antibodies targeting AcSTn. Out of 18 mice immunized with PSM, 5 developed antibodies specific for GcSTn, 2 developed antibodies specific for AcSTn and 10 developed antibodies that were pan-STn-specific. Out of 17 mice immunized with OSM, none developed antibodies that were GcSTn-specific, 11 developed antibodies that were AcSTn-specific and 12 developed antibodies that were pan-STn-specific.

The effect of different adjuvant/antigen combinations on wild type mouse body weight was also assessed. Group 5 mice received PSM (100 μg)+CFA or IFA (100 μl), Group 6 mice received PSM (10 μg)+AbiSCO-100 (12 μg)+ODN-2395 (100 μg), Group 7 mice received OSM (100 μg)+CFA or IFA (100 μl) and Group 8 mice received OSM (10 μg)+AbiSCO-100 (12 μg)+ODN-2395 (100 μg). Body weights were obtained daily during the study. Results are presented in the following Table.

TABLE 11
Body weight changes in response to antigen/adjuvant

| | Average body weight (g) by group, | | | |
|---|---|---|---|---|
| | standard deviations in parenthesis | | | |
| | Group | Group | Group | Group |
| Day | 5 | 6 | 7 | 8 |
| −3 | 18.2 | 18.2 | 18.2 | 18.2 |
| | (0.8) | (0.8) | (0.7) | (0.7) |
| 0 | 17.8 | 18 | 18.1 | 18.1 |
| | (0.5) | (0.6) | (0.9) | (0.7) |
| 3 | 17.3 | 14.7 | 16.5 | 15 |
| | (0.6) | (0.8) | (0.9) | (0.6) |
| 7 | 18.6 | 18.2 | 19 | 18.4 |
| | (1.1) | (0.9) | (0.8) | (0.6) |
| 10 | 19.3 | 18.2 | 19.8 | 18.4 |
| | (0.6) | (0.5) | (0.9) | (0.5) |
| 14 | 20.4 | 18.6 | 20.4 | 18.8 |
| | (0.8) | (0.7) | (1.1) | (0.5) |
| 17 | 20.4 | 17.6 | 20.1 | 17.5 |
| | (0.8) | (0.6) | (1.1) | (0.9) |
| 21 | 21.1 | 19.2 | 21 | 19.3 |
| | (0.7) | (0.5) | (1) | (0.6) |
| 24 | 20.9 | 19.4 | 21.1 | 19.5 |
| | (0.9) | (0.8) | (1.1) | (0.4) |
| 28 | 21.4 | 20.1 | 21.9 | 20.1 |
| | (1) | (0.9) | (1.4) | (0.5) |

Mice receiving immunizations with AbISCO-100+ODN-2395 (Groups 6 and 8) demonstrated reduced body weight in comparison with mice receiving immunizations with CFA/IFA (Groups 5 and 7). Differences between mice receiving PSM (Groups 5 and 6) versus OSM (Groups 7 and 8) were not substantial.

### Example 4. Glycan Array Analysis

Optimized glycan arrays comprise 71 chemically synthesized and well-defined glycans, most of which comprise Neu5Ac and Neu5Ge glycan pairs. Array slides are obtained commercially (Arraylt Corp, Sunnyvale, CA) and include the glycans listed in the following Table.

TABLE 12
Array glycans

| Glycan | |
|---|---|
| ID No. | Glycan |
| 1 | Neu5,9Ac2α2,3Galβ1,4GlcNAcβO(CH2)2CH2NH2 |
| 2 | Neu5Gc9Acα2,3Galβ1,4GlcNAcβO(CH2)2CH2NH2 |
| 3 | Neu5,9Ac2α2,6Galβ1,4GlcNAcβO(CH2)2CH2NH2 |
| 4 | Neu5Gc9Acα2,6Galβ1,4GlcNAcβO(CH2)2CH2NH2 |
| 5 | Neu5Acα2,6GalNAcαO(CH2)2CH2NH2 |
| 6 | Neu5Gcα2,6GalNAcαO(CH2)2CH2NH2 |
| 7 | Neu5,9Ac2α2,3Galβ1,3GlcNAcβO(CH2)2CH2NH2 |
| 8 | Neu5Gc9Acα2,3Galβ1,3GlcNAcβO(CH2)2CH2NH2 |
| 9 | Neu5,9Ac2α2,3Galβ1,3GalNAcαO(CH2)2CH2NH2 |
| 10 | Neu5Gc9Acα2,3Galβ1,3GalNAcαO(CH2)2CH2NH2 |
| 11 | Neu5Acα2,3Galβ1,4GlcNAcβO(CH2)2CH2NH2 |
| 12 | Neu5Gcα2,3Galβ1,4GlcNAcβO(CH2)2CH2NH2 |
| 13 | Neu5Acα2,3Galβ1,3GlcNAcβO(CH2)2CH2NH2 |
| 14 | Neu5Gcα2,3Galβ1,3GlcNAcβO(CH2)2CH2NH2 |
| 15 | Neu5Acα2,3Galβ1,3GalNAcαO(CH2)2CH2NH2 |
| 16 | Neu5Gcα2,3Galβ1,3GalNAcαO(CH2)2CH2NH2 |
| 17 | Neu5Acα2,6Galβ1,4GlcNAcβO(CH2)2CH2NH2 |
| 18 | Neu5Gcα2,6Galβ1,4GlcNAcβO(CH2)2CH2NH2 |
| 19 | Neu5Acα2,6Galβ1,4GlcβO(CH2)2CH2NH2 |
| 20 | Neu5Gcα2,6Galβ1,4GlcβO(CH2)2CH2NH2 |
| 21 | Neu5Acα2,3Galβ1,4GlcβO(CH2)2CH2NH2 |
| 22 | Neu5Gcα2,3Galβ1,4GlcβO(CH2)2CH2NH2 |
| 23 | Neu5,9Ac2α2,6GalNAcαO(CH2)2CH2NH2 |
| 24 | Neu5Gc9Acα2,6GalNAcαO(CH2)2CH2NH2 |
| 25 | Neu5Acα2,3GalβO(CH2)2CH2NH2 |
| 26 | Neu5Gcα2,3GalβO(CH2)2CH2NH2 |
| 27 | Neu5Acα2,6GalβO(CH2)2CH2NH2 |
| 28 | Neu5Gcα2,6GalβO(CH2)2CH2NH2 |
| 29 | Neu5,9Ac2α2,3GalβO(CH2)2CH2NH2 |
| 30 | Neu5Gc9Acα2,3GalβO(CH2)2CH2NH2 |
| 31 | Neu5,9Ac2α2,6GalβO(CH2)2CH2NH2 |
| 32 | Neu5Gc9Acα2,6GalβO(CH2)2CH2NH2 |
| 33 | Neu5Acα2,3Galβ1,3GalNAcβO(CH2)2CH2NH2 |
| 34 | Neu5Gcα2,3Galβ1,3GalNAcβO(CH2)2CH2NH2 |
| 35 | Neu5,9Ac2α2,3Galβ1,3GalNAcβO(CH2)2CH2NH2 |
| 36 | Neu5Gc9Acα2,3Galβ1,3GalNAcβO(CH2)2CH2NH2 |
| 37 | Neu5,9Ac2α2,6Galβ1,4GlcβO(CH2)2CH2NH2 |
| 38 | Neu5Gc9Acα2,6Galβ1,4GlcβO(CH2)2CH2NH2 |
| 39 | Neu5,9Ac2α2,3Galβ1,4GlcβO(CH2)2CH2NH2 |
| 40 | Neu5Gc9Acα2,3Galβ1,4GlcβO(CH2)2CH2NH2 |
| 41 | Neu5Acα2,8Neu5Acα2,3Galβ1,4GlcβO(CH2)2CH2NH2 |
| 42 | Neu5Acα2,8Neu5Acα2,8Neu5Acα2,3Galβ1, |
| | 4GlcβO(CH2)2CH2NH2 |
| 43 | Galβ1,4GlcβO(CH2)2CH2NH2 |
| 45 | Galβ1,4GlcNAcβO(CH2)2CH2NH2 |
| 47 | GalNAcαO(CH2)2CH2NH2 |
| 51 | Galβ1,3GalNAcβO(CH2)2CH2NH2 |
| 52 | Galβ1,3GlcNAcαO(CH2)2CH2NH2 |
| 53 | Galβ1,3GlcNAcβO(CH2)2CH2NH2 |
| 54 | Galβ1,4GlcNAc6SβO(CH2)2CH2NH2 |
| 55 | Neu5Acα2,3Galβ1,4(Fucα1,3)GlcNAcβO(CH2) |
| | 2CH2NH2 |
| 56 | Neu5Gcα2,3Galβ1,4(Fucα1,3)GlcNAcβO(CH2) |
| | 2CH2NH2 |
| 57 | Neu5Acα2,3Galβ1,4(Fucα1,3)GlcNAc6SβO(CH2) |
| | 2CH2NH2 |
| 58 | Neu5Gcα2,3Galβ1,4(Fucα1,3)GlcNAc6SβO(CH2) |
| | 2CH2NH2 |
| 59 | Galβ1,3GlcNAcβ1,3Galβ1,4GlcβO(CH2)2CH2NH2 |
| 60 | Neu5Acα2,3Galβ1,3GlcNAcβ1,3Galβ1,4GlcβO(CH2) |
| | 2CH2NH2 |
| 61 | Neu5Gcα2,3Galβ1,3GlcNAcβ1,3Galβ1,4GlcβO(CH2) |
| | 2CH2NH2 |
| 62 | Neu5Acα2,3Galβ1,4GlcNAc6SβO(CH2)2CH2NH2 |
| 63 | Neu5Gcα2,3Galβ1,4GlcNAc6SβO(CH2)2CH2NH2 |
| 64 | Neu5Acα2,8Neu5Acα2,3Galβ1,4GlcβO(CH2) |
| | 3NHCOCH2(OCH2CH2)6NH2 |
| 65 | Neu5Acα2,8Neu5Acα2,8Neu5Acα2,3Galβ1, |
| | 4GlcβO(CH2)3NHCOCH2(OCH2CH2)6NH2 |
| 66 | Neu5Acα2,6(Neu5Acα2,3)Galβ1,4GlcβO(CH2)2CH2NH2 |
| 67 | Neu5Acα2,6(Neu5Gcα2,3)Galβ1,4GlcβO(CH2)2CH2NH2 |
| 68 | Neu5Acα2,6(KDNα2,3)Galβ1,4GlcβO(CH2)2CH2NH2 |
| 69 | Neu5Gcα2,8Neu5Acα2,3Galβ1,4GlcβO(CH2)2CH2NH2 |
| 70 | KDNα2,8Neu5Acα2,3Galβ1,4GlcβO(CH2)2CH2NH2 |
| 71 | Neu5Acα2,8Kdnα2,6Galβ1,4GlcβO(CH2)2CH2NH2 |
| 72 | Neu5Acα2,8Neu5Gcα2,3Galβ1,4GlcβO(CH2)2CH2NH2 |
| 73 | Neu5Acα2,8Neu5Gcα2,6Galβ1,4GlcβO(CH2)2CH2NH2 |
| 74 | KDNα2,8Neu5Gcα2,3Galβ1,4GlcβO(CH2)2CH2NH2 |
| 75 | Neu5Gcα2,8Neu5Gcα2,3Galβ1,4GlcβO(CH2)2CH2NH2 |
| 76 | Neu5Acα2,8Neu5Acα2,6Galβ1,4GlcβO(CH2)2CH2NH2 |

300 ml of epoxy blocking buffer is prepared by combining 15 ml of 2 M Tris buffer (pH 8) with 0.9 ml of 16.6 M ethanolamine and 284.1 ml of distilled water. The solution was brought to a final pH of 9.0 with HCl. The solution is filtered using a 0.2 μM nitrocellulose membrane. The epoxy buffer solution as well as 1 L of distilled water are pre-warmed to 50° C. Glass slides are arranged in a slide holder and quickly submerged in a staining tub with the warmed epoxy blocking buffer. Slides are incubated in the epoxy blocking buffer for 1 hour at 50° C. with periodic shaking to deactivate epoxy binding sites. Next, slides are rinsed and blocked with PBS with 1% OVA at 25° C. for one hour. Serum samples with polyclonal antibodies (1:1000) or purified monoclonal antibodies (1 ug/mL), are diluted in PBS with 1% OVA and added to the glycan array for one hour at 25° C. After extensive washing, binding of antibodies are detected by incubating glycan microarray slides with Cy3-conjugated anti-mouse IgG (Jackson Immunoresearch, West Grove, PA) for one hour. Slides are then washed extensively, dried and scanned with a Genepix 4000B scanner (Laser at 100%; gain at 350; 10 μm pixels). Raw data from scanned images are extracted using the Genepix software and analysis of raw data is carried out. Antibodies are considered to be highly specific for AcSTn and GcSTn if they demonstrate binding to both molecules, but not to Tn or any other glycans on the array.

### Example 5. Antibodies Derived from Group 6 Mice

Serum samples from group 6 mice were harvested on day 56 after immunization and were subjected to anti-STn serum titer analysis according to Example 2. Mice #'s 4401 and 4436 demonstrated an anti-STn serum titer of 1:62,500 and were selected for glycan-interacting profiling according to the glycan array of Example 4.

Serum of mouse #4401 showed strong specificity for AcSTn and GcSTn and the 9-O-acetylated variants. Minimal binding towards O-linked Ac-α2,6Gal and Gc-α2,6Gal was also observed. Serum from mouse #4436 showed high specificity for AcSTn and GcSTn and their 9-O-acetylated variants with minimal binding towards O-linked Ac-α2,6Gal and Gc-α2,6Gal. On the basis of these data, both mice #'s 4401 and 4436 were selected for hybridoma fusion. A final boost with 100 μg of PSM administered by intraperitoneal injection was given on day 78 after immunization and fusion was carried out with harvested spleens 3 days after.

For hybridoma formation, splenocytes from each of the two mice were fused separately to SP2/0 mouse myeloma hybrid cells and seeded at very low density (˜10,000 cells/well; 30×96-well plates/fusion) to obtain monoclonal antibodies without the need for subcloning. MRC-5 human embryonic fibroblast cell lines were used as feeder cells. Anti-STn BSM ELISA was carried out according to the method described in Example 2 to screen resulting hybridomas. The first supernatant screenings were performed without periodate treatment. A second screening was carried out after periodate treatment to identify clones with anti-STn-specific binding. After the second screening, only two clones, 18D2 and 18C7, both derived from mouse #4436, were found to be specific for STn.

### Example 6. Validation and Characterization of 18D2 and 18C7 Antibodies

18D2 and 18C7 clones were expanded and binding properties of these hybridomas were determined by glycan microarray analysis according to Example 4. Each antibody showed reactivity to AcSTn (mean fluorescence intensity of about 16,000 for 18D2 and about 9,000 for 18C7,) GcSTn (mean fluorescence intensity of about 7,000 for 18D2 and about 3,500 for 18C7,) Neu5,9Ac2-α2,6-GalNAc (mean fluorescence intensity of about 4,500 for 18D2 and about 4,500 for 18C7) and Neu5,9Ac5Gc-α2,6-GalNAc (mean fluorescence intensity of about 4,000 for 18D2 and about 2,000 for 18C7). No binding to Tn antigen or other glycans was detected.

Binding affinities of 18D2 and 18C7 to STn were compared to commercially available anti-STn antibodies using the BSM ELISA assay described in Example 2. Commercial anti-STn antibodies used for comparison included 3F1 (SBH Sciences, Natick, MA,) TAG-72 clone B72.3 (Thermo Fisher Scientific, Waltham, MA,) TAG-72 clone CC49 (Santa Cruz Biotechnology, Santa Cruz, CA) as well as a negative control antibody against c-myc (Thermo Fisher Scientific, Waltham, MA). Each antibody was titrated using a 7-point, 1:5 serial dilution starting from 100 nM. Titrated antibodies were added to BSM ELISA plates containing wells that were non-treated or treated with sodium periodate. Antibody binding was determined using HRP-conjugated polyclonal goat anti-mouse IgG antibody (Jackson Immunoresearch Laboratories, West Grove, PA). The reaction was developed using an HRP substrate and stopped with 1.6 M sulfuric acid. Optical densities were measured at 490 nm using a Spectramax microplate reader. Results indicated that both 18D2 and 18C7 bound non-treated wells and did not bind to periodate-treated wells, suggesting that the antibodies are specific for STn and do not bind Tn. Additionally, 18D2 antibodies were found to bind with higher affinity than 18C7 antibodies. The half maximal effective concentration (EC50) for each antibody was calculated using a non-linear regression analysis carried out on periodate subtracted data. B72.3, CC49 and 3F1 antibodies showed the highest affinities with EC50 values below 1 nM. 18D2 demonstrated an EC50 of 6.3 nM, while 18C7 had an EC50 of 62.5 nM.

18D2 and 18C7 antibodies were further characterized by ELISA comprising antigens other than BSM. ELISAs were carried out according to Example 2, with the exception that ELISA plates were coated with biotinylated polyacrylamide (PAA) particles comprising either AcSTn (AcSTn-PAA), GcSTn (GcSTn-PAA) or Tn (Tn-PAA) as antigens. These particles present the antigens in a multivalent format. Briefly, 96-well ELISA plates were coated with 200 ng/well of each PAA antigen and blocked with PBS with 1% OVA. Antibodies were serially diluted at 1:5 in PBS with 1% OVA using an initial concentration of 1 μM. After blocking, antibodies were added to the plates and plates were incubated for 1.5 hours at room temperature. Binding of anti-STn antibodies was detected using a goat anti-mouse IgG-HRP conjugated antibody (Jackson Immunoresearch Laboratories, West Grove, PA). The reaction was developed by addition of HRP substrate and hydrogen peroxide. The reaction was then stopped by the addition of sulfuric acid (1.6 M). Optical densities were measured at 490 nm. A dose-response curve was generated using readings at each concentration tested and a non-linear regression curve was generated with four-parameter modeling to determine EC50 values for each antibody.

Binding affinities of 18D2 and 18C7 antibodies were compared to 3F1, B72.3 and CC49 as well as to anti-c-myc as a negative control. 18D2 bound to AcSTn-PAA with high affinity (EC50=1.71 nM) while 18C7 had lower affinity (EC50=4.1 nM). B72.3, CC49 and 3F1 demonstrated EC50 values of 71.32 nM, 156.9 nM and 152.2 nM respectively. Similar results were obtained with GcSTn-PAA assays, yielding EC50 values of 1.57 nM and 2.59 nM for 18D2 and 18C7 respectively and values of 27.81 nM, 31.49 nM and 73.17 nM for B72.3, CC49 and 3F1 respectively. Binding of antibodies to Tn-PAA was very weak for all of the antibodies tested.

### Example 7. Sequencing of 18D2 and 18C7 Antibodies

Variable domain as well as full heavy and light chain sequences were determined for 18D2 and 18C7 clones generated from mouse number 4436. This was carried out by extracting total RNA from the hybridoma cells, performing reverse-transcriptase (RT)-PCR to amplify antibody sequences, identifying positive clones by gel electrophoresis and cloning and sequencing positive DNA. Sequences obtained are listed in Tables 2, 3 and 4.

### Example 8. Flow Cytometry-Based Analysis of Antibody Binding

Flow cytometry-based analysis is carried out to elucidate the curve-dose response for binding of antibodies to cell surface antigens. For these analyses, three cell lines are employed.

MDA-MB-231 cells are human breast cancer cells. They are grown in Earle's Minimum Essential Medium supplemented with 10% fetal calf serum (FCS), 100 μg/ml penicillin, 100 UI/ml streptomycin and 45 μg/ml gentamycin. MCF-7 cells are also human breast cancer cells and are grown under the same conditions as MDA-MB-231 cells. Stably transfected versions of MDA-MB-231 and MCF-7 cells (clone TAH3.P10 for MDA-MB-231 cells and clone A12.1 for MCF-7 cells) that over express GalNAc α2,6-sialyltransferase (ST6GalNAc 1,) are also cultured under the same conditions with the exception of an added 1 mg/ml of G418 to support cells expressing the transgene. ST6GalNAc 1 is an enzyme capable of sialylating GalNAc. As a result of over expression, transfected cells express high levels of Neu5Ac-STn (see Julien, S. et al., Glycoconjugate journal. 2001. 18, 883-93; the contents of which are herein incorporated by reference in their entirety). E3 cells are murine breast cancer cells. They are cultured in Dulbecco's E4 medium with 10% FCS. Stably transfected versions of E3 cells expressing high levels of Neu5Gc-STn (E3-STn) are cultured with 600 μg/ml of G418 and 200 μg/ml hygromycin. During growth and maintenance of experimental cells, trypsin is not used for cell passaging.

For analysis, cells are harvested using StemPro Accutase (Life Technologies, Carlsbad, CA) and washed with PBS comprising 5% FBS before pelleting by light centrifugation. Cell numbers and viability are determined by trypan blue dye exclusion analysis and cell concentrations are adjusted to 5×10⁶ cells/ml in PBS with 5% FBS. 50 μl of cells are added to each well of an assay plate. Cells are combined with 50 μl solutions of antibody being analyzed or control antibodies and incubated for 1 hour at 4° C. Cells are washed and pelleted twice with PBS with 5% FBS before being treated with 100 μl of PBS with 5% FBS comprising a 1:1,500 dilution of anti-mouse IgG (Southern Biotech, Birmingham, Alabama,) conjugated to allophycocyanin (APC). Cells are incubated for 30 min at 4° C. before washing and resuspending in 200 μl of propidium iodide (PI) diluted 1:1000 in PBS with 5% FBS. Treated cells are then subjects to flow cytometry analysis and 10,000 events are acquired for each sample.

### Example 9. Flow Cytometry-Based Analysis of 18D2 and 18C7 to Confirm and Assess Binding to STn

Binding of 18D2 and 18C7 antibodies to cell-associated STn was assessed by flow cytometry-based analysis according to Example 8. In addition to 18D2 and 18C7 antibodies, 3F1 antibody (SBH Biosciences, Natick, MA) and 9E10.3 c-myc antibody (Thermo Fisher Scientific, Waltham, MA) were used as controls. 18D2 was found to bind to ST6GalNAc 1 transfected MDA-MB-231 cells with an EC50 of 44.08 nM. 18C7 demonstrated an EC50 of 60.35 nM and 3F1 demonstrated an EC50 of 2.42 nM.

### Example 10. Antibodies Derived from Group 8 Mice

Serum samples from group 8 mice were harvested and subjected to anti-STn serum titer analysis according to Example 2. Based on the results, serum samples from mice #'s 4406 and 4407 were selected for glycan-interacting profiling according to the glycan array of Example 4 and hybridoma generation.

Mouse #4406 received a final boost with 100 μg of OSM administered by intraperitoneal injection on day 85 after immunization and fusion was carried out with harvested spleens 3 days after. Mouse #4407 received a final boost of 100 μg OSM antigen on day 77 (without adjuvant) and fusion was carried out with harvested spleens 3 days after.

For hybridoma formation, splenocytes from each of the two mice were fused separately to SP2/0 mouse myeloma hybrid cells and seeded at very low density (˜10,000 cells/well; 30×96-well plates/fusion) to obtain monoclonal antibodies without the need for subcloning. MRC-5 human embryonic fibroblast cell lines were used as feeder cells. Anti-STn BSM ELISA was carried out according to the method described in Example 2 to screen resulting hybridomas. The first supernatant screenings were performed without periodate treatment. A second screening was carried out after periodate treatment to identify clones with anti-STn-specific binding. After the second screening, clone 10A5 derived from mouse #4406 and clones 8C11, 2D4 and 7G9 from mouse #4407, were found to be specific for STn.

### Example 11. Flow Cytometry-Based Analysis of Antibodies Produced by Group 8 Mice

Binding of 2D4 and 8C11 antibodies to cell-associated STn was assessed by flow cytometry-based analysis according to Example 8. In addition to 2D4 and 8C11 antibodies, 18D2 and S3F (internally produced anti-STn antibody, IgG2a) antibodies were tested and all were compared to 9E10.3 c-myc antibody (Thermo Fisher Scientific, Waltham, MA) as a negative control. Antibodies produced by 2D4 subclones 2D4-1B4 and 2D4-2E2 were found to bind to ST6GalNAc 1 transfected MDA-MB-231 cells with high affinity with an EC50 of 1.19 nM and 2.23 nM, respectively. 8C11 antibodies had lower affinity with an EC50 of 7.52 nM, while 18D2 had an EC50 of 74.51 and S3F demonstrated an EC50 of 2.051 nM.

Flow cytometry-based analysis was also carried out according to the method of Example 8 to characterize binding of antibodies produced by clones 10A5 and 7G9 to ST6GalNAc 1 transfected MDA-MB-231 cells. Antibodies produced by 10A5 clones had high affinity for cell-associated STn (EC50 value of 3.62 nM,) 7G9 antibodies had a lower affinity (EC50 of 6.91 nM) and S3F antibodies, in comparison, had an EC50 value of 2.13 nM.

### Example 12. Sequence Analysis of Antibodies from Clones 10A5, 8C11, 2D4 and 7G9

Variable domain sequences for antibodies produced by selected hybridomas generated from mouse number 4407 were obtained. This was carried out by extracting total RNA from hybridoma cells, performing reverse-transcriptase (RT)-PCR to amplify heavy chain and light chain variable domains, identifying positive clones by gel electrophoresis and cloning and sequencing positive DNA. Variable domain sequences and CDR sequences obtained from the analysis are presented in Tables 2, 3 and 4.

### Example 13. Antibody Characterization Summary

Based on characterization data, anti-STn antibodies developed from Group 6 and Group 8 mice were categorized by anti-STn group number. In some cases, antibodies were assigned to Group 1. Group 1 antibodies according to the invention are antibodies capable of binding AcSTn and GcSTn. Such antibodies may have the ability to associate with a wider range of STn structures. The large oval in FIG. 1A indicates the portion of STn recognized by Group 1 antibodies. Antibodies were assigned to Group 2 based on their ability to bind STn as well as some related structures that include an O-linkage to serine or threonine. The large oval in FIG. 1B indicates the portion of STn recognized by Group 2 antibodies. In some cases, Group 2 antibodies may bind to glycans comprising a sialylated galactose residue. Some Group 2 antibodies preferably bind to structures with AcSTn over structures with GcSTn. Further anti-STn antibodies were assigned to Group 3. Group 3 antibodies are antibodies capable of binding STn, but may also bind a broader set of related structures. Unlike Group 2 antibodies, Group 3 antibodies do not require that such structures have an O-linkage to serine or threonine. The large oval in FIG. 1C indicates the portion of STn recognized by Group 3 antibodies. Finally, some anti-STn antibodies were assigned to Group 4. Group 4 antibodies are capable of binding to both AcSTn and GcSTn as well as the un-sialylated Tn antigen, and therefore have broader specificity. The large oval in FIG. 1D indicates the portion of STn recognized by Group 4 antibodies. The following Table shows the antibody groups in which antibodies were assigned based on flow cytometry analysis and glycan array analysis. Also listed in the Table is the antibody Isotype for each antibody as determined by the IsoStrip mouse monoclonal antibody isotyping kit (Roche Diagnostics GmbH, Manheim, Germany).

TABLE 13
Anti-STn antibodies and their antibody groups

| | | Flow cytometry (EC50) | | Glycan Array | | | |
|---|---|---|---|---|---|---|---|
| Antibody | Isotype | MDA-STn+ | MDA-STn− | AcSTn | GcSTn | STn Specificity | Group |
| 2D4 | IgG2b κ | 1.41 | NB | 88% | 1% | 89% | Group 2 |
| 7G9 | IgG2b κ | 5.15 | NB | 72% | 7% | 79% | Group 2 |
| 10A5 | IgG2c κ | 5.57 | NB | 96% | 1% | 97% | Group 2 |
| 8C11 | IgG2b κ | 7.28 | NB | 75% | 9% | 84% | Group 2 |
| 18C7 | IgG2c κ | 353.5 | NB | 60% | 40% | 100% | Group 1 |
| 18D2 | IgG2b κ | 65.78 | NB | 50% | 50% | 100% | Group 1 |

### Example 14. Antibody Internalization Study on STn+ and STn− Cells

6-well culture dishes were prepared with collagen-coated coverslips in the center of 4 wells of each dish. Wells were seeded with either 10⁵ cells/well of MDA-MD-231 cells or with 10⁶ cells/well of MDA-MD-231 STn cells (expressing STn) and cultured for 24 hours.

10 μg/ml antibody solutions were prepared in cell culture media. A separate solution was prepared for each of LA22 antibody (EMD Millipore, Burlington, MA) as a positive control for cell surface binding, S3F antibody (internal reagent) as a positive control for STn binding, 8C11, 2D4, 7G9, 10A5 and GB26.16 (QED Bioscience, San Diego, CA) as a negative control. Media were removed from wells and 100 μl of each antibody solution was added to separate coverslips. Coverslips were covered with parafilm and incubated for 30 minutes at 4° C. to allow antibody binding. Antibody solutions were removed by washing and fresh media was added to each well. Cells were incubated at 37° C. for 15, 30, 60 or 240 min to allow for antibody internalization.

After incubation, cells were prepared for microscopy. Cells were washed with PBS and fixed with paraformaldehyde fixation buffer (PFA) containing 3% paraformaldehyde and 2% sucrose in PBS for 15 minutes at room temperature. Cells were rinsed again in PBS and treated with blocking buffer made up of PBS with 1% bovine serum albumin (BSA) or with permeabilization buffer made up of blocking buffer with 0.1% TX-100. Cells were incubated in for 30 min at room temperature, rinsed in PBS and treated with secondary antibody (ALEXA FLUOR® 488-labeled goat-anti-mouse IgG) for 2 hours at room temperature. Cells were rinsed again in PBS and treated with DAPI nuclear stain for 5 min at room temperature in the dark. Cells were rinsed again in PBS and mounted for fluorescence microscopy. Net cell counts of cells showing positive internalization for each antibody at each time point are presented in the following Table.

TABLE 14
Cell counts for cells showing antibody internalization

| Time | 10A5, | 2D4, | S3F, | 7G9, | 8C11, | LA22, | GB26, |
|---|---|---|---|---|---|---|---|
| (min) | STn+ | STn+ | STn+ | STn+ | STn+ | STn− | STn+ |
| 15 min | 267 | 1340 | 426 | 458 | 934 | 230 | |
| 30 min | 346 | 413 | 410 | 154 | 297 | | |
| 60 min | 75 | 533 | 350 | 126 | 122 | 39 | |
| 4 hr | 93 | 325 | 259 | 61 | 49 | 53 | 15 |
| 4 hr | 97 | 536 | 276 | 145 | 160 | 49 | 34 |
| no perm | | | | | | | |

Antibodies 2D4 and 8C11 showed the greatest levels of internalization.

### Example 15. Flow Cytometry Analysis of Antibody Internalization

Flow cytometry analysis is carried out in order to quantify the extent of antibody internalization according to the procedure of Example 8, with several notable distinctions.

For analysis, stably transfected variants of MDA-MB-231 cells (clone TAH3.P10) that express high levels of cell surface-bound Neu5Ac-STn are harvested using 10 mM EDTA and washed with PBS comprising 1% BSA before pelleting by light centrifugation. Cell numbers and viability are determined by trypan blue dye exclusion analysis and cell concentrations are adjusted to 5×10⁶ cells/ml in PBS with 1% BSA. 50 μl of cells are added to each well of an assay plate. Cells are combined with 50 μl solutions of antibody or fluorescently-labeled antibody and incubated for 1 hour at 4° C. Following this incubation period, cells are washed with PBS to remove unbound antibody and aliquots are removed for incubation for various times (15, 30, 60 minutes) at 37° C. to allow bound antibody to internalize at a physiologically relevant temperature. After each incubation, cell surface-bound antibody is removed by treating cells with acidic medium (150 mM NaCl, pH=2.5) Cells treated with unlabeled antibody are washed with PBS and fixed with paraformaldehyde fixation buffer (PFA) containing 3% paraformaldehyde and 2% sucrose in PBS for 15 minutes at room temperature. These cells are rinsed again in PBS and treated with blocking buffer made up of PBS with 1% bovine serum albumin (BSA). Cells are incubated for 30 min at room temperature, rinsed in PBS and treated with secondary antibody (allophycocyanin-labeled goat-anti-mouse IgG) for 2 hours at room temperature. All cells are then washed with PBS and subjected to flow cytometry analysis wherein 10,000 events are recorded for each sample. Residual fluorescent signal in acid-treated samples is further quenched via treatment with trypan blue dye.

### Example 16. Flow Cytometry Assay to Quantify Antibody Internalization

Flow cytometry analysis was carried out in order to quantify the extent of antibody internalization according to the procedure of Example 15. MDA-MB-231 cells expressing high levels of cell-surface bound Neu5Ac-STn were harvested using StemPro Accutase buffer and washed with PBS comprising 5% FBS before pelleting by light centrifugation. Cell numbers and viability were determined by trypan blue dye exclusion analysis and cell concentrations were adjusted to 5×10⁶ cells/ml in PBS with 5% FBS. 50 μl of cells were added to each well of an assay plate. Cells were then combined with 50 μl solutions of antibody S3F or ALEXA FLUOR® 488-conjugated antibodies S3F, 7G9 or GB26.6 and incubated for 1 hour at 4° C. Following this incubation period, cells were washed with PBS to remove unbound antibody and aliquots were removed for incubation for various times (15, 30, 60 minutes) at 37° C. After each incubation, cells were treated with acidic medium (150 mM NaCl, pH=2.5) to remove surface-bound antibody. Cells treated with unlabeled S3F were washed with PBS 5% FCS. Secondary antibody (allophycocyanin-labeled goat-anti-mouse IgG) was added to the cells and incubated for 30 min at 4° C., rinsed in PBS 5% FBS and kept on ice until flow cytometry analysis. All cells were then washed with PBS and subjected to flow cytometry analysis wherein 10,000 events were recorded for each sample.

Flow cytometry analysis revealed that approximately 98.6% of a cell sample treated with unlabeled antibody S3F exhibited fluorescence following incubation at 4° C., while 50-57% of cell samples treated with antibody S3F-ALEXA FLUOR® 488 conjugates exhibited fluorescence after the staining period (see Table below). The proportion of fluorescent cells in this population dropped to less than 10% following treatment with acidic media, indicating that incubation in acidic buffer effectively removed cell-surface bound antibody. Following incubation at 37° C., cells that were treated with S3F-ALEXA FLUOR® 488 conjugate and subjected to acidic conditions continued to exhibit fluorescence, as 22-32% of cells from these samples retained fluorescence following acid treatment. In a separate experiment, between 20-60% of cells from these samples exhibited fluorescence after treatment with acidic buffer. Additionally, samples of cells from these populations that were treated with trypan blue to quench residual fluorescence from surface-bound antibody contained populations of between 20 and 60% that continued to exhibit fluorescence. Samples of cells that had been treated with ALEXA FLUOR® 488-conjugated GB26.6 were found to have less than 2% of fluorescent cells following staining and internalization periods, in the presence and absence of acid. Collectively, these data indicate that both antibody S3F and S3F-ALEXA FLUOR® 488 conjugate are internalized, while antibody GB26.6 is not effectively taken up by the Neu5Ac-STn-overexpressing MDA-MB-231 cell line.

Analysis of the cellular uptake of ALEXA FLUOR® 488-conjugated antibody 7G9 using flow cytometry revealed that approximately 80% of cells stained with this conjugate at 4° C. exhibited fluorescence prior to acidic treatment, and this proportion dropped only marginally to approximately 70% following acidic treatment. However, treatment of samples of these cells with trypan blue dye resulted in a population of less than 20% fluorescent cells (see Table below). Cells that were treated with ALEXA FLUOR® 488-conjugate 7G9 and allowed to incubate at 37° C. for various times exhibited populations of between 60-80% fluorescent cells prior to acid treatment and trypan blue exposure, and the proportions of cells retaining a fluorescent signal following treatment with acidic buffer and trypan blue dye were between 20-45%. These data indicate that ALEXA FLUOR® 488-conjugated antibody 7G9 was successfully internalized by this cell line.

TABLE 15
Optimization of flow cytometry quantification of antibody internalization

| | | | | | | % cells | % cells |
|---|---|---|---|---|---|---|---|
| | | | | Inc. | | exhibiting | exhibiting |
| | | | | Time | | APC | 488 |
| 1° Ab | Conj. | 2º Ab | Temp | 37° C. | Acid | fluorescence | fluorescence |
| N/A | | | | | | 0.67 | 0.45 |
| S3F | | anti-APC | 4° C. | | − | 98.6 | |
| S3F | 488 | N/A | 4° C. | | − | | 57.9 |
| S3F | 488 | N/A | 4° C. | | + | | 9.29 |
| S3F | 488 | N/A | 4° C., | 15′ | − | | 56.6 |
| | | | then 37° C. | | | | |
| S3F | 488 | N/A | 4° C., | 30′ | | | 53.1 |
| | | | then 37° C. | | | | |
| S3F | 488 | N/A | 4° C., | 60′ | − | | 50.9 |
| | | | then 37° C. | | | | |
| S3F | 488 | N/A | 4° C., | 15′ | + | | 22.5 |
| | | | then 37° C. | | | | |
| S3F | 488 | N/A | 4° C., | 30′ | + | | 25.9 |
| | | | then 37° C. | | | | |
| S3F | 488 | N/A | 4° C., | 60′ | + | | 31.5 |
| | | | then 37° C. | | | | |
| GB26.6 | 489 | N/A | 4° C. | | − | | 0.62 |
| GB26.6 | 489 | N/A | 4° C. | | + | | 0.37 |
| GB26.7 | 490 | N/A | 4° C., | 60′ | − | | 1.47 |
| | | | then 37° C. | | | | |
| GB26.7 | 490 | N/A | 4° C., | 60′ | + | | 0.38 |
| | | | then 37° C. | | | | |

In the Table, Ab refers to antibody, Conj. refers to conjugate and Inc. refers to incubation.

### Example 17. 8C11 Antibody Internalization

Binding of 8C11 and irrelevant control antibodies (16101, 16102, MCP11) to STn expressed on MDA cells was assessed by flow cytometry-based analysis according to Example 8.

The 8C11 antibody under investigation in this study (Lot 111113) was found to bind to STn expressed on MDA cells with an EC50 of 7.28 nM. The EC50 of 8C11 Lot 111113 was similar to the prior 8C11 Lot 1416-859789 which was found to bind to STn expressed on MDA cells with an EC50 of 7.52 nM. No binding of 8C11 to MDA parental cells was observed

Irrelevant control antibodies 16101, 16102, and MCP11 were found to bind to MDA cells expressing STn at high concentrations of antibody. MCP11 at a concentration of 100 nm was found to bind to 9.9% of MDA cells expressing STn. However, the mean fluorescence intensity (MFI) of the APC-conjugated secondary to the anti-STn antibodies was very low at all concentrations of antibody tested, indicating weak binding by irrelevant control antibodies 16101, 16102, and MCP11.

Irrelevant control antibodies 16102, and MCP11 did not bind to parental MDA cells lacking expression of STn at high concentrations of antibody. Irrelevant control antibody 16101 at a high concentration of 300 nM was found to weakly bind to 3.7% of MDA cells lacking expression of STn.

### Example 18. Flow Cytometry Characterization of Antibody Binding

Binding of S3F IgG2a, 7G9 #1, 7G9 #2, 7G9 #3, 2D4 #1, 2D4 #2, 10A5 #1, 10A5 #2, and GB26.6 (anti-gentamicin antibody) to STn expressed on MDA cells was assessed by flow cytometry analysis according to Example 8 (with “#1,” “#2,” and “#3” representing different lots of recombinantly expressed antibody). The S3F antibody was used as a positive control and the GB26.6 antibody was used as a negative control for binding STn on MDA cells. The 2D4 antibody was found to bind to STn expressed on MDA cells with high affinity as characterized by an EC50 of 1.41 nM compared to the positive control S3F antibody EC50 value of 1.94 nM (see the following Table). The 7G9 #3, 10A5, and 7G9 #2 antibodies were characterized by lower affinity with EC50 values of 5.15 nM, 5.57 nM, and 7.33 nM respectively. EC50 values determined by flow cytometry were calculated based on the mean fluorescence intensity of the APC fluor.

TABLE 16
Antibody EC50 values

| Antibody | EC50 value |
|---|---|
| S3F | 1.94 |
| 7G9#1 | 6.91 |
| 7G9#2 | 7.33 |
| 7G9#3 | 5.15 |
| 2D4#1 | 2.23 |
| 2D4#2 | 1.41 |
| 10A5#1 | 3.62 |
| 10A5#2 | 5.57 |

### Example 19. Evaluate Antibody Internalization Through Cell Viability Assay

Cell viability assays are performed to screen anti-STn antibodies of the present invention in the presence and absence of secondary antibody-drug conjugates (2°ADCs). The purpose of the screen is to identify the ability of each anti-STn antibody to inhibit cell growth. Antibodies with potent cell growth inhibition are used to design direct antibody-drug conjugates (ADCs). Using such secondary antibody-drug conjugates (2°ADCs) in cell-based cytotoxic assays can quickly pre-screen many ADC candidates against tumor cells. Based on the assay, a naked antibody candidate is directly added to cells in the presence of a 2°ADC. Internalization of the mAb/2°ADC complex into cells that express a high density of the targeted antigen can achieve a dose-dependent drug release within the cells, causing a cytotoxic effect to kill the cells (e.g., tumor cells), while cells expressing a low density of the targeted antigen are not affected (e.g., normal cells).

To perform cell viability assays, cell lines described in the present application (MDA-MB-231 parental, MDA-MB-231-STn+, and OV-90) are prepared and cultured for the assays. The cell culture is optimized for cell density by plating different densities of cells (e.g., 2,000, 4,000 and 7,500 per well) on a 96-well plate and observing the cell growth for 96 hours. The plating condition in which cells reach around 90% confluence at the end of the 96 hours is identified and the optimal cell number is then used in the final viability assay.

Antibodies are tested in one or more cell lines in the presence and absence of a 2°ADC such as Fab αMFc-CL-MMAF. Duplicate or triplicate cell plates for each cell line are used for testing each antibody candidate.

For cell viability assays, data points are collected for each antibody candidate with duplicates for each data point. Each antibody candidate is diluted in serial concentrations from 0.3 pM to 20 nM. A constant amount of Fab αMFc-CL-MMAF (40 nM) is used in the viability assay.

Alternatively, data points are collected for each antibody candidate with triplicates for each data point. Each antibody candidate is diluted in serial concentrations from 1 pM to 20 nM. A constant amount of Fab αMFc-CL-MMAF (40 nM) is used in the viability assay.

Cell viabilities are measured by Cell-Titer Glo luminescence based assays.

### Example 20. Internalization of Anti-STn Antibodies

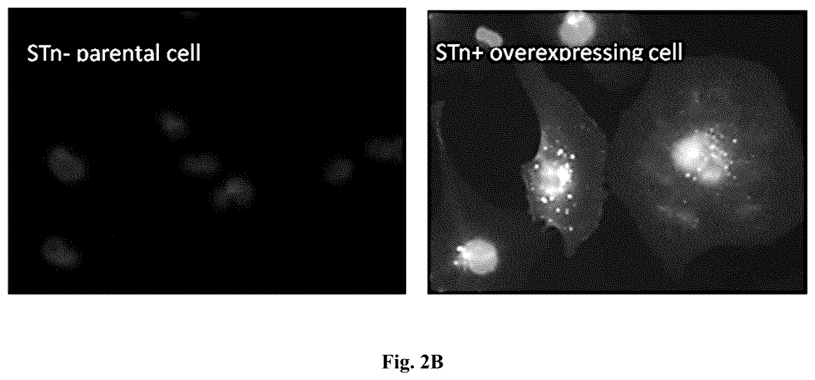

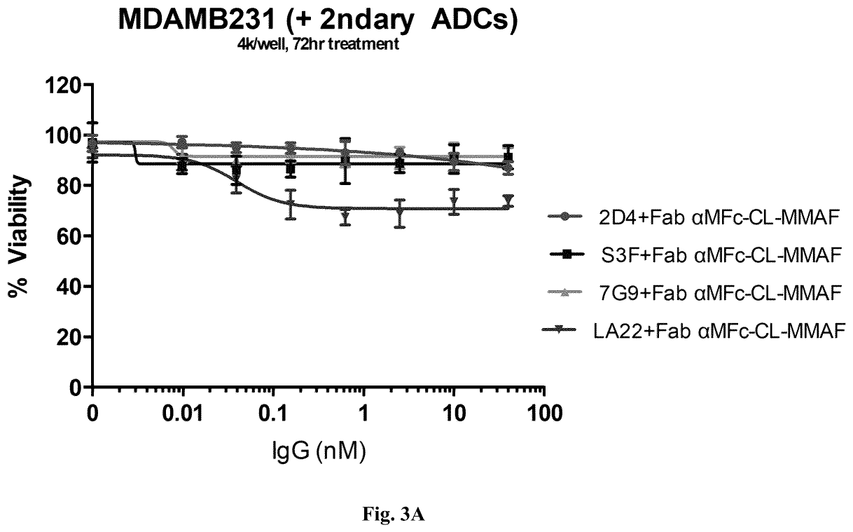

S3F can be internalized into cells as shown by FACS assay and immunofluorescence staining. FIGS. 2A and 2B show the internalization of S3F and the cellular distribution of S3F inside cells.

Cell viability assays were used to further test the antibody internalization according to the method described in Example 19.

Three anti-STn monoclonal antibodies (anti-STn antibodies S3F, 7G9 and 2D4) and an anti-EGFR monoclonal antibody LA22 were tested for internalization. Six plates of cell culture, two per cell line (MDA-MB-231 parental, MDA-MB-231-STn+, and OV-90), were used to assay the antibody candidates. Each plate can be used to assay the four antibody candidates simultaneously. A secondary anti-IgG antibody conjugated to MMAF (monomethyl auristatin phenylalanine), a non-permeable Aurastatin analog, was used as the 2°ADC. Cell viability was then measured using CellTitreGlo Luminescent Cell Viability Assay (Promega, Madison, WI).

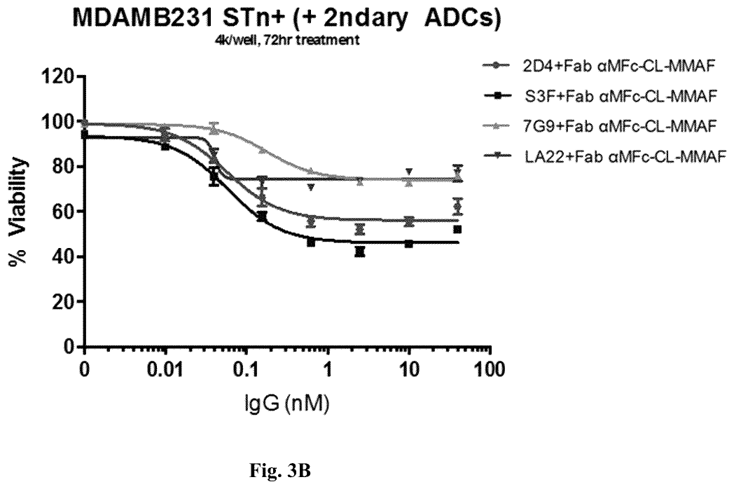

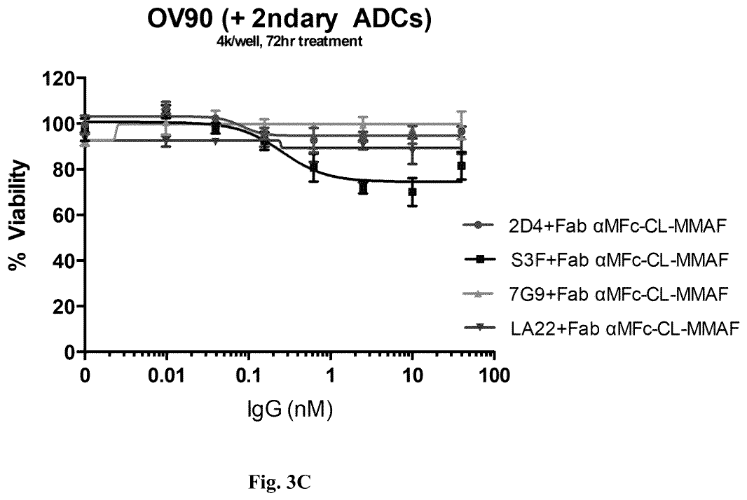

The assay results (presented in the following Table) indicate that anti-STn antibodies S3F, 7G9 and 2D4 demonstrate activity in MDA-MB-231-STn+ cells, but not MDA-MB-231 parental cells. FIG. 3 depicts the cell viabilities for each anti-STn antibody in the presence of Fab αMFc-CL-MMAF in MDA-MB-231 parental cells (FIG. 3A) and MDA-MB-231-STn+ cells (FIG. 3B). The anti-EGFR antibody LA22 was used in the assay as a positive control.

TABLE 17
IC50 of anti-STn antibodies in secondary internalization assays in
MDA-MB-231-STn+ cells

| Antibody | |
|---|---|
| candidate | IC50 (nM) |
| 2D4 | 0.056 |
| S3F | 0.058 |
| 7G9 | 0.1811 |
| LA22 | 0.04 |

It was also observed that only S3F demonstrated modest activity in OV-90 cells in the presence of Fab αMFc-CL-MMAF as shown in FIG. 3C. The anti-EGFR antibody LA22 was used in the assay as a negative control since there is no EGFR expression in OV-90 cells. The lack of effect of 7G9 and 2D4 may be due to the lack of sensitivity to the cytotoxic drug MMAF or due either to lack of internalization or not enough receptor density to internalize enough antibodies. Assays with different concentrations of antibodies, increased cell densities and/or different cytotoxic drug conjugates may be used to test the antibody internalization.

In another cell viability assay in the presence of the DNA damaging agent DMSA (dimercaptosuccinic acid), S3F demonstrated improved activity in MDA-MB-231-STn+ cells, as compared to the microtubule inhibitor MMAF. The IC50 in the presence of αMFc-CL-DMSA was 0.2430, while the IC50 in the presence of Fab αMFc-CL-MMAF was only 0.1243.

### Example 21. Demonstration of In Vivo Tumor Killing Ability

In vivo tumor killing ability is demonstrated with mouse and/or human tumor cell lines. Tumor cell lines expressing STn targets are transferred into mice and the ability of the antibody candidates to kill the resulting tumors is determined.

Mouse cell lines used in vivo in tumor killing assays include the mouse colon adenocarcinoma cell line, MC38, derived from C57BL/6 mice and stably transfected with ST6 (alpha-N-acetyl-neuraminyl-2,3-beta-galactosyl-1,3)-N-acetylgalactosaminide alpha-2,6-sialyltransferase 1 (ST6GalNac1). These cells are fed with sialic acid (Neu5Ac and/or Neu5Gc, depending on target) before their use in in vivo tumor killing assays using syngeneic Cmah^{-/-} mice.

Alternatively, for in vivo tumor killing assays human breast cancer cell lines (T47-D, MCF-7 or MDA-MB-231) induced to express a high level of STn are transferred into immune-deficient FOXN1−/−(nude) cells, non-obese diabetic (NOD) cells, or severe immunodeficiency (SCID) mice.

In vivo ADCC is induced by passive transfer of human peripheral blood mononuclear cells (PBMCs) or purified natural killer (NK) cells. In cases where candidate antibodies bind unspecifically to wild-type mouse tissue, immune-deficient mice are bred into the Cmah−/− background.

### Example 22 Antibody Evaluation by the E3-STn Transfected Murine Breast Tumor Allograft Model

Antibody anti-tumor activity was tested using a murine breast tumor allograft model. Balb/c mice were used as part of the test system. 50 female mice were inoculated with E3-STn cells to cause STn-expressing tumor formation. Mice were maintained under pathogen-free conditions with irradiated feed and autoclaved water. Each mouse received 2.5×10⁵ cells in a 1:1 mixture of MATRIGEL® (BD Biosciences, Franklin Lakes, New Jersey) to media (0.1 ml total volume) by injection into the inguinal mammary fat pad. Beginning on the first day of the study, body weight and tumor volumes were recorded twice weekly. 33 mice with mean tumor volume between 100 and 150 mm³ were selected for randomization.

3 test articles were evaluated during the study. 25 mg/kg of 7G9-1A8 IgG2b κ antibody was administered as a 5 mg/ml solution in PBS (Life Technologies, Carlsbad, CA) and was evaluated along with vehicle-only (PBS) control. 7G9-1A8 antibodies comprise the 7G9-1A8 heavy chain variable domain sequence (SEQ ID NO: 12) with a light chain variable domain sequence (SEQ ID NO: 9) that is identical to others developed from Group 8 mice. Antibodies were administered by intraperitoneal injection, twice weekly for 3 weeks (on days 1, 5, 8, 12, 15 and 19 of the study). 15 mice received 7G9-1A8 antibodies while 10 mice were administered vehicle-only. An additional 8 mice received 10 mg/ml of Paclitaxel (a mitotic inhibitor used in cancer treatment,) daily for 5 days.

At the end of the study, all tumor samples were collected from each mouse by way of necropsy. Tumors were bisected with one half being preserved by formalin fixation followed by paraffin embedding and the other half being snap frozen in optimum cutting temperature (OCT) compound.

Average tumor volumes (in mm³) calculated on days 1, 4, 6, 8, 11, 13, 15, 18, 20, 22, 26, 27, 29, 32, 34, 36, 39 and 41 following the first treatment with each test article are presented in the following Table. Standard deviation values are listed in parenthesis.

TABLE 18
Average tumor volumes

| | Average Tumor Volume (mm³) | | |
|---|---|---|---|
| Days of | Vehicle | 7G9-1A8 | |
| Treatment | Control | Antibody | Paclitaxel |
| 1 | 112.3 | 112.4 | 112.7 |
| | (18.8) | (21.8) | (27.2) |
| 4 | 157.9 | 148.7 | 179.5 |
| | (32.8) | (36.9) | (78.2) |
| 6 | 193.4 | 161.9 | 191.4 |
| | (37.8) | (32.6) | (80.2) |
| 8 | 215.9 | 152.8 | 198.6 |
| | (40.8) | (38.3) | (80.6) |
| 11 | 243.2 | 136.3 | 199.8 |
| | (51.9) | (39.2) | (109.4) |
| 13 | 261.9 | 118.4 | 227.0 |
| | (65.4) | (32.1) | (134.6) |
| 15 | 271.2 | 105.1 | 248.4 |
| | (78.7) | (30.8) | (136.1) |
| 18 | 292.8 | 99.5 | 290.5 |
| | (88.4) | (31.5) | (219.1) |
| 20 | 307.8 | 91.7 | 328.2 |
| | (99.0) | (37.3) | (262.0) |
| 22 | 315.0 | 105.2 | 331.8 |
| | (106.3) | (31.5) | (260.5) |
| 26 | 320.3 | 102.8 | 344.0 |
| | (114.3) | (34.6) | (287.1) |
| 27 | 320.4 | 107.4 | 350.1 |
| | (114.3) | (36.4) | (303.7) |
| 29 | 332.9 | 147.1 | 352.3 |
| | (118.9) | (70.6) | (314.7) |
| 32 | 328.1 | 160.6 | 355.1 |
| | (124.2) | (81.2) | (322.3) |
| 34 | 327.4 | 164.7 | 358.4 |
| | (125.3) | (84.0) | (325.2) |
| 36 | 346.9 | 212.1 | 282.0 |
| | (131.0) | (133.9) | (137.9) |
| 39 | 339.1 | 206.6 | 267.2 |
| | (133.9) | (147.0) | (142.6) |
| 41 | 343.7 | 235.3 | 276.4 |
| | (135.8) | (179.1) | (145.5) |

Average tumor volumes in mice treated with 7G9-1A8 were greatly reduced in mice receiving 7G9-1A8 antibodies as compared to Paclitaxel and vehicle control. Interestingly, tumor volumes rose steadily after day 22 of the study, when 7G9-1A8 antibody treatment was stopped

The average weight of mice in each treatment group was also determined on days 1,4, 6, 8,11, 13, 15, 18, 20, 22, 26, 27, 29, 32, 34, 36, 39 and 41 following the first administration of each test article (see the following Table). The percent gain or loss over initial weight values is listed in parentheses.

TABLE 19
Average weight

| | Average Weight (g) | | |
|---|---|---|---|
| Day of | Vehicle | 7G9-1A8 | |
| Study | Control | Antibody | Paclitaxel |
| 1 | 17.1 | 17.1 | 16.7 |
| 4 | 17.3 | 17.3 | 16.5 |
| | (1.1) | (1.7) | (−1.1) |
| 6 | 17.6 | 18.2 | 17 |
| | (2.8) | (6.9) | (1.8) |
| 8 | 17.7 | 18.2 | 17.3 |
| | (3.4) | (6.5) | (3.8) |
| 11 | 17.8 | 18.3 | 17.1 |
| | (3.9) | (7.3) | (2.9) |
| 13 | 18.0 | 18.6 | 17.4 |
| | (5.0) | (8.9) | (4.2) |
| 15 | 18.4 | 18.7 | 17.8 |
| | (7.8) | (9.9) | (6.8) |
| 18 | 18.3 | 18.6 | 17.6 |
| | (7.3) | (9.0) | (5.6) |
| 20 | 18.4 | 18.9 | 17.8 |
| | (7.3) | (10.8) | (7.0) |
| 22 | 18.6 | 18.9 | 17.9 |
| | (8.6) | (11.1) | (7.4) |
| 26 | 19.0 | 19.0 | 18.3 |
| | (10.9) | (11.7) | (9.8) |
| 27 | 18.9 | 19.1 | 18.5 |
| | (10.6) | (11.9) | (10.9) |
| 29 | 19.3 | 19.4 | 18.9 |
| | (13.1) | (14.0) | (13.7) |
| 32 | 19.2 | 19.2 | 18.9 |
| | (12.0) | (12.6) | (13.5) |
| 34 | 19.6 | 19.5 | 19.2 |
| | (14.7) | (14.2) | (15.1) |
| 36 | 19.6 | 19.6 | 18.5 |
| | (14.5) | (14.8) | (11.2) |
| 39 | 20.3 | 20.2 | 19.1 |
| | (18.6) | (18.7) | (14.5) |
| 41 | 20.5 | 20.3 | 19.2 |
| | (20.0) | (19.2) | (15.1) |

Mice treated with 7G9-1A8 displayed a higher percent gain in weight over the course of treatment as compared to Paclitaxel and vehicle control. Interestingly, this effect was diminished after antibody treatments were stopped.

At the end of the study, a complete blood count (CBC) was obtained for 5 mice treated with antibody as well as 3 mice treated with vehicle control. Blood was collected from all mice via terminal cardiac puncture and processed for plasma. Blood was placed in EDTA microtainer tubes (BD & Co., Franklin Lakes, NJ) then centrifuged. Plasma layers were then removed and snap frozen in cryo vials (Thermo-Fisher Scientific, Rochester, NY) and stored at −80° C. until analysis. Analysis was carried out to look for levels of a variety of factors. Results of the analysis are presented in the following Table.

TABLE 20
CBC results

| | | Control | | 7G9-1A8 | | Typical |
|---|---|---|---|---|---|---|
| Factor | Units | Level | St. Dev. | Level | St. Dev. | Range ± St. Dev. |
| White blood cells | 10³/L | 20.4 | 6.1 | 13.9 | 5.3 | 6.7 ± 2.1 |
| Lymphocytes | 10⁹/L | 7.8 | 2.8 | 5.6 | 0.7 | N/A |
| Monocytes | 10⁹/L | 1.4 | 0.2 | 0.7 | 0.6 | N/A |
| Neutrophils | 10⁹/L | 11.2 | 3.6 | 7.6 | 4.6 | N/A |
| % Lymphocytes | % | 38.0 | 5.3 | 44.4 | 13.8 | 70.3 ± 9.3 |
| % Monocytes | % | 7.4 | 2.9 | 4.6 | 3.3 | 2.9 ± 2.6 |
| % Neutrophils | % | 54.6 | 3.6 | 51.0 | 11.7 | 24.7 ± 8.6 |
| (Segmented) | | | | | | |
| Red blood cells | 10⁶/L | 10.4 | 0.2 | 9.9 | 0.7 | 9.1 ± 0.6 |
| Hemoglobin | g/dL | 16.1 | 0.2 | 16.0 | 0.3 | 15.3 ± 0.8 |
| Hematocrit | % | 50.1 | 1.3 | 46.9 | 2.4 | 47.6 ± 4.5 |
| Mean Corpuscular | fL | 48.3 | 2.1 | 47.4 | 1.1 | 52.4 ± 2.3 |
| Volume | | | | | | |
| Mean Corpuscular | pg | 15.4 | 0.3 | 16.2 | 0.9 | 16.9 ± 0.7 |
| Hemoglobin | | | | | | |
| Mean Corpuscular | g/dL | 32.1 | 0.9 | 34.2 | 1.3 | 32.4 ± 2.3 |
| Hemoglobin | | | | | | |
| Concentration | | | | | | |
| Red Cell | % | 19.7 | 0.2 | 19.0 | 0.4 | 14.9 ± 1.6 |
| Distribution | | | | | | |
| Width | | | | | | |
| Platelet count | 10³ | 801.7 | 42.8 | 712.2 | 81.5 | 784.8 ± 210.6 |
| Procalcitonin | % | 0.6 | 0.1 | 0.5 | 0.1 | N/A |
| Mean platelet | fL | 7.4 | 0.4 | 7.4 | 0.3 | 6.3 ± 0.6 |
| volume | | | | | | |
| Platelet Cell | % | 33.0 | 1.3 | 32.2 | 0.5 | N/A |
| Distribution | | | | | | |
| Width | | | | | | |

Values obtained between control and antibody treated-mice were not found to vary significantly, indicating that antibody treatments have a low probability of toxicity.

### Example 23. Pathological Evaluation

At the end of the study described in Example 22, three mice from the vehicle control group and five mice that received 7G9-1A8 were chosen at random for post-study pathology assessment. Brain, colon, intestine, heart, kidney, liver, lung, mandibular salivary gland, pancreas, spleen, stomach, adrenal gland and thyroid gland organs were collected from each mouse. The organs were fixed in formalin (VWR; Radnor, PA) for about 48 hours, transferred to 70% ethanol (Sigma-Aldrich; St. Louis, MO), and then processed for standard H&E staining.

The H&E stained tissue slides were examined for pathology review. Liver sections from each animal had multifocal inflammation with or without concomitant hepatocellular necrosis. The histo-pathological changes were minimal to mild in severity. There was no appreciable difference in the morphology of lesions among vehicle control group mice and mice that received 7G9-1A8. Mild histo-pathological severity was noted only in the vehicle control group mice, however the numbers of mice examined and the similar, albeit less severe, change noted in the mice that received 7G9-1A8 suggested there was no significant difference between the groups.

Epicardial inflammation was observed in two vehicle control group mice, although the cause was unknown. A similar epicardial inflammation was not observed in the mice that received 7G9-1A8.

Aggregates of primarily neutrophils in the muscularis mucosa of the stomach was observed most prominently in the non-glandular areas of two vehicle control group mice, although the cause was unknown. A similar aggregation in the muscularis mucosa of the stomach was not observed in the mice that received 7G9-1A8.

### Example 24. Antibody Evaluation by the E3-STn Transfected Murine Breast Tumor Allograft Model (Second Round Study)

As described in Example 22, a second round experiment was carried out to further validate antibodies. 50 female Balb/cmice were inoculated with E3-STn cells to cause STn-expressing tumor formation when they were 5 weeks old. Mice were housed in individually ventilated microisolator cages, and maintained under pathogen-free conditions with irradiated feed and autoclaved water. Each mouse received 2.5×10⁵ cells in a 1:1 mixture of MATRIGEL® (BD Biosciences, Franklin Lakes, New Jersey) to media (0.1 ml total volume) by injection into the inguinal mammary fat pad. Beginning on the first day of the study, body weight and tumor volumes were recorded twice weekly. 33 mice with mean tumor volume between 100 and 150 mm³ were selected for randomization. Study ended at Day 50 with vehicle group having mean tumor volume of 469 mm³ and antibody treated group having mean tumor volume of 313 mm³.

25 mg/kg of 7G9-1A8 IgG2b x antibody was administered as a 5 mg/ml solution in vehicle (20 mM Citrate (pH5.5) and 150 mM NaCl) and was evaluated along with vehicle-only control. 7G9-1A8 antibody comprise the 7G9-1A8 heavy chain variable domain sequence (SEQ ID NO: 12) with a light chain variable domain sequence (SEQ ID NO: 9) that is identical to others developed from Group 8 mice. Antibodies and vehicle control were administered by intraperitoneal injection, twice weekly for 7 weeks.

At the end of study, blood was collected from all mice in all groups at time of termination via terminal cardiac puncture and processed for serum. Blood was placed in a serum separator tube (Becton & Dickinson Co.; Franklin Lakes, NJ), centrifuged, then serum was transferred to a cryovial (VWR; Radnor, PA) and snap frozen in liquid nitrogen before storage at −80° C.

At the end of the study, all tumor samples were collected from each mouse by way of necropsy. Tumors were bisected with one half being preserved by formalin fixation followed by paraffin embedding and the other half being snap frozen in optimum cutting temperature (OCT) compound.

Average tumor volumes (in mm³) calculated on days 1, 5, 8, 12, 15,19, 22, 26, 29, 33, 36, 40, 43, 47 and 50 following the first treatment with each test article are presented in the following Table. Standard deviation values are listed in parenthesis.

TABLE 21
Average tumor volumes

| | Average Tumor Volume (mm³) | |
|---|---|---|
| Days of | Vehicle | 7G9-1A8 |
| Treatment | Control | Antibody |
| 1 | 126.1 | 125.9 |
| | (14.0) | (15.1) |
| 5 | 150.2 | 135.2 |
| | (15.11) | (17.7) |
| 8 | 175.1 | 144.5 |
| | (18.6) | (18.4) |
| 12 | 201.1 | 157.2 |
| | (45.0) | (18.8) |
| 15 | 235.4 | 169.2 |
| | (59.2) | (32.7) |
| 19 | 265.6 | 178.0 |
| | (70.6) | (44.6) |
| 22 | 285.8 | 186.4 |
| | (74.4) | (56.5) |
| 26 | 300.6 | 195.9 |
| | (76.2) | (58.0) |
| 29 | 325.7 | 199.4 |
| | (74.5) | (64.1) |
| 33 | 337.0 | 201.5 |
| | (75.4) | (67.1) |
| 36 | 362.8 | 206.5 |
| | (73.9) | (74.8) |
| 40 | 380.7 | 212.9 |
| | (71.1) | (81.9) |
| 43 | 402.7 | 243.7 |
| | (71.7) | (84.2) |
| 47 | 424.6 | 277.5 |
| | (78.5) | (87.6) |
| 50 | 469.1 | 313.4 |
| | (96.2) | (81.5) |

Average tumor volumes in mice treated with 7G9-1A8 were greatly reduced in mice receiving 7G9-1A8 antibodies as compared to vehicle control. The average weight of mice in each treatment group was also determined on days 1, 5, 8, 12, 15,19, 22, 26, 29, 33, 36, 40, 43, 47 and 50 following the first administration of each test article (see the following Table). The percent gain or loss over initial weight values is listed in parentheses.

TABLE 22
Average body weight

| | Average Weight (g) | |
|---|---|---|
| Day of | Vehicle | 7G9-1A8 |
| Study | Control | Antibody |
| 1 | 17.8 | 18.3 |
| 5 | 18.0 | 18.7 |
| | (1.0) | (2.4) |
| 8 | 18.9 | 19.4 |
| | (5.8) | (6.5) |
| 12 | 18.7 | 20.1 |
| | (4.7) | (10.1) |
| 15 | 18.9 | 20.4 |
| | (6.1) | (12.0) |
| 19 | 19.3 | 20.9 |
| | (8.1) | (14.3) |
| 22 | 19.3 | 20.8 |
| | (8.4) | (14.1) |
| 26 | 19.5 | 21.5 |
| | (9.3) | (17.8) |
| 29 | 19.9 | 22.1 |
| | (11.4) | (21.1) |
| 33 | 19.8 | 22.2 |
| | (11.1) | (21.4) |
| 36 | 20.2 | 22.1 |
| | (13.2) | (21.0) |
| 40 | 20.5 | 22.8 |
| | (15.0) | (25.0) |
| 43 | 20.9 | 22.9 |
| | (17.5) | (25.4) |
| 47 | 21.2 | 22.8 |
| | (18.7) | (25.0) |
| 50 | 21.9 | 23.8 |
| | (22.7) | (30.2) |

Mice treated with 7G9-1A8 displayed a higher percent gain in weight over the course of treatment as compared to vehicle control. Interestingly, this effect was diminished after antibody treatments were stopped.

### Example 25. Phage Library Construction and Selection

RNA is prepared from spleens harvested from mice with a strong immune response to immunization. Mouse variable (V) regions are PCR amplified and assembled into scFv expression constructs. ScFv sequences are cloned into phagemid display vectors allowing for scFv display on the surface of M13 phage particles. The resulting library is transformed into E. coli (TG1). Bulk transformations of E. coli are grown and phage are prepared by phage rescue. In the first round of selection, phage from the culture medium are purified by PEG precipitation.

Candidate scFvs are selected using both negative and positive selection methods. For negative selection, the library is incubated with “destroyed” STn-negative mucin (e.g. chemically treated PSM). For positive selection, the library is incubated with GcSTn mucin (e.g. PSM and/or de-O-acetylated BSM), AcSTn mucin (e.g. OSM and/or de-O-acetylated BSM) or BSM (and/or de-O-acetylated BSM) and a synthetic glycan (Neu5Gc and/or Neu5Ac) in the presence of a Neu5Ac or Neu5Gc (depending on the desired target).

After 3-4 rounds of selection with reducing antigen concentrations, 1000 clones are analyzed by ELISA for binding to STn (e.g. Neu5Ac and/or Neu5Gc) using synthetic and natural glycan targets. Lead phage/scFv candidates are tested in a secondary flow cytometry-based cellular assay for binding to GcSTn and/or AcSTn using Jurkat cells with or without “induction” of GcSTn or AcSTn. Up to 20 selected scFv candidates of interest are subjected to further analysis.

Lead scFv candidates are selected for conversion to IgG. Variable regions from each scFv are cloned into mammalian expression vectors between an upstream CMV promoter and a downstream immunoglobulin constant region. Heavy chain vector includes murine IgG1 and x constant regions. Vectors are transiently transfected into HEK293/EBNA cells. Antibody samples are purified and characterized by binding to positive and negative glycan epitopes. Samples of up to 0.5 mg of each whole IgG are further analyzed.

### Example 26. Antibody-Dependent Cell-Mediated Cytotoxicity Optimization

Genes encoding the variable regions of a selected IgG are cloned into mammalian expression vectors encoding human Fc regions (huIgG1κ) containing amino acid mutations known to enhance Fc-receptor binding and antibody-dependent cell-mediated cytotoxicity (ADCC). Vectors are transiently transfected into HEK293/EBNA cells. After 2-7 days, IgG expression is quantified and samples of antibody are purified on protein A columns. Antibodies are then tested in ADCC assays. Neu5Gc and Neu5Ac-expressing Jurkat cell lines are used as the target cells and human peripheral blood mononuclear cells (PBMC) are used as a source of effector cells. Target cells are titrated using maximum cell lysis to determine the optimum cell density for use in multiwall plate format assay. ADCC-mutated antibody together with the non-mutated IgG are pre-incubated with target cells, effector cells are then added at varying target:effector cell ratios, and cultures are incubated at 37° C. Percentage viability is determined using Calcein-AM dye (BD Biosciences, San Jose, CA) release. Samples of up to 0.5 mg of ADCC-mutated IgG are subjected to further analysis.

### Example 27. Production of Lead Antibody from Semi-Stable HEK Cell Line

Variable regions from IgG are cloned into mammalian expression vectors between an upstream CMV promoter and a downstream immunoglobulin constant region. Heavy chain vector includes murine IgG1 and x constant regions. Vectors are transiently transfected into HEK293/EBNA cells and antibody titers are assessed at 72 hours. Transiently transfected HEK293/EBNA cells are selected with hygromycin to establish a semi-stable expression system. Semi-stable cells are expanded to 10 liters. Antibodies are purified from the culture supernatant by Protein A, dialyzed into PBS and the resulting preparation is analyzed for (1) aggregates by analytical size exclusion chromatography (SEC), (2) endotoxin levels by Limulus amebocyte lysate (LAL) testing (expressed as EU/mg), and (3) binding to antigen in the primary assay.

### Example 28. Additional Assays for Screening scFv Candidates for Target Affinity

ScFv candidates are subjected to additional screening methods for STn (pan-STn, AcSTn and/or GcSTn) affinity using a variety of proposed targets.

### Synthetic Glycan Target Screening

As used herein, the term “target screening” refers to the use of a target substance to identify binding partners for that substance. Synthetic glycan target screening is carried out using desired STn target antigens bound to poly(acrylic acid) (PAA) with a biotin tag. Undesired STn target antigens as well as Tn bound to PAA with a biotin tag are used as negative controls. Cells associated with candidate scFvs are isolated through precipitation with avidin-associated entities.

### Natural Glycan Target Screening on Live Cells

Target screening using live cells is carried out using Jurkat cells fed with sialic acid (Neu5Gc and/or Neu5Ac, depending on the desired antibody target) or Jurkat cells fed with an alternative form of sialic acid (Neu5Gc and/or Neu5Ac, depending on the desired antibody target) as a negative control. Target screening using live cells is also carried out using MCF-7 or MDA-MB-231 cells fed with sialic acid (Neu5Gc and/or Neu5Ac, depending on the desired antibody target or whether being used for negative control screening) and stable transfection. Flow cytometry is used in either case to isolate cells associated with scFv candidates.

### Natural Glycan Target Screening on Tissue (Ex Vivo)

Target screening using ex vivo tissue is carried out using biopsy tissue samples. Binding of scFv candidates with ex vivo tissue is analyzed using standard immunohistochemical methods. Single tissue sections as well as tissue microarray sections are used. Samples are treated with or without sialidase and/or periodate in control experiments.

### Example 29. Antibody Humanization

Fully humanized heavy and light chains are designed. Protein models of the variable regions are generated using existing antibody structures as templates. Segments of starting heavy and light chain variable region amino acid sequences are compared with human sequences for possible inclusion in the fully humanized sequences. Series of humanized heavy and light chain variable regions are designed entirely from segments of human variable region sequences with the objective that T cell epitopes be avoided. Variant human sequence segments with significant incidence of potential T cell epitopes as determined by in silico technologies are discarded.

Humanized heavy and light chain variable region genes are constructed from overlapping oligonucleotides assembled into full length genes using the ligase chain reaction (LCR). LCR products are amplified and suitable restriction sites are added for cloning into expression vectors. PCR products are cloned into intermediate vectors and confirmed by sequencing.

For construction of expression plasmids encoding fully humanized antibodies with human constant regions, DNA sequences for each variable region are inserted into mammalian expression vectors between an upstream cytomegalovirus immediate/early promoter/enhancer (CMV IE) plus the immunoglobulin signal sequence and a downstream immunoglobulin constant region gene. DNA samples are prepared for transfection into mammalian cells.

For generation of cell lines and selection of lead fully humanized antibodies, heavy and light chain plasmid DNA pairs are transfected into mammalian cells (NS0). Cell lines producing humanized antibodies are expanded and antibody samples are purified. Antibodies are tested in primary and secondary binding assays to determine leading antibody candidates. The 3 leading candidates are used for further analysis.

### Example 30. Immunogenicity Testing

Lead antibodies are subjected to EpiScreen (Antitope, Paradise Valley, AZ) whole antibody human T cell assays using a minimum of 20 blood samples from healthy volunteer donors. Immunogenicity of lead antibodies is compared with control chimeric antibodies with starting antibody variable regions and matched human constant regions. Data are benchmarked against EpiScreen whole protein data for clinical-stage biologics.

### Example 31. Cell Line Development

Cell lines are developed with the ability to yield high levels of antibody with no non-human glycosylation due to knock down of the CMAH gene. Cell lines are glycoengineered to increase ADCC. These cell lines have the ability to perform in small and large scale production.

### Example 32. Antibody-Dependent Inhibition of STn-Positive Tumor Cell Immune Tolerance

Anti-STn antibodies of the present invention are provided and used to contact tumor cells and tissues comprising STn glycans. Immune-dependent targeting of STn-tumor cells is increased.

### Example 33. Treatment of Immune Tolerant Tumors Using Anti-STn Antibodies

A subject with STn glycans expressed on and around tumor cells is treated with an anti-STn antibody. Immune tolerance of subject tumor cells is decreased.

### Example 34. Generation of S3F Antibodies

S3F IgG2a antibodies were generated through the combination of 3F1 IgG1 variable domains (SBH Biosciences, Natick, MA) with antibody constant domain regions from IgG2 antibodies. The heavy and light chain variable domains of 3F1 were sequenced and constructs were generated encoding 3F1 variable domains upstream of IgG2 expression vectors, plasmid H1206 (LakePharma, Belmont, CA) for antibody heavy chains and plasmid L1206 (LakePharma, Belmont, CA) for antibody light chains. Related sequences are presented in the following Table.

Table 23. Sequences utilized in S3F IgG2 antibody generation

TABLE 23
Sequences utilized in S3F IgG2 antibody generation

| | | SEQ |
|---|---|---|
| | | ID |
| Description | Sequence | NO |
| 3F1 VH | QVQLQQSDAELVKPGASVKISCKASGYTFTDHAIHWVKQK | 59 |
| domain | PEQGLDWIGYISPGNGDIKYNEKFKDKVTLTADKSSSTACM | |
| | HLNSLTSEDSAVYFCKRSLLALDYWGQGTTLTVSS | |
| 3F1 VL | DIVMTQSHKFMSTSVGDRVSITCKASQDVGTNIAWYQQKP | 60 |
| domain | GRSPKVLIYSASTRHTGVPDRFTGSGSGTDFTLTISNVQSED | |
| | LTDYFCQQYSSFPLTFGVGTKLELK | |
| IgG2a heavy | AKTTAPSVYPLAPVCGDTTGSSVTLGCLVKGYFPEPVTLTW | 61 |
| chain | NSGSLSSGVHTFPAVLQSDLYTLSSSVTVTSSTWPSQSITCN | |
| constant | VAHPASSTKVDKKIEPRGPTIKPCPPCKCPAPNLLGGPSVFIF | |
| domain | PPKIKDVLMISLSPIVTCVVVDVSEDDPDVQISWFVNNVEV | |
| | HTAQTQTHREDYNSTLRVVSALPIQHQDWMSGKEFKCKV | |
| | NNKDLPAPIERTISKPKGSVRAPQVYVLPPPEEEMTKKQVT | |
| | LTCMVTDFMPEDIYVEWTNNGKTELNYKNTEPVLDSDGSY | |
| | FMYSKLRVEKKNWVERNSYSCSVVHEGLHNHHTTKSFSRT | |
| | PGK | |
| kappa light | RADAAPTVSIFPPSSEQLTSGGASVVCFLNNFYPKDINVKW | 62 |
| chain | KIDGSERQNGVLNSWTDQDSKDSTYSMSSTLTLTKDEYER | |
| constant | HNSYTCEATHKTSTSPIVKSFNRNEC | |
| domain | | |
| S3F full | QVQLQQSDAELVKPGASVKISCKASGYTFTDHAIHWVKQK | 63 |
| length heavy | PEQGLDWIGYISPGNGDIKYNEKFKDKVTLTADKSSSTACM | |
| chain | HLNSLTSEDSAVYFCKRSLLALDYWGQGTTLTVSSAKTTA | |
| | PSVYPLAPVCGDTTGSSVTLGCLVKGYFPEPVTLTWNSGSL | |
| | SSGVHTFPAVLQSDLYTLSSSVTVTSSTWPSQSITCNVAHPA | |
| | SSTKVDKKIEPRGPTIKPCPPCKCPAPNLLGGPSVFIFPPKIK | |
| | DVLMISLSPIVTCVVVDVSEDDPDVQISWFVNNVEVHTAQT | |
| | QTHREDYNSTLRVVSALPIQHQDWMSGKEFKCKVNNKDL | |
| | PAPIERTISKPKGSVRAPQVYVLPPPEEEMTKKQVTLTCMV | |
| | TDFMPEDIYVEWTNNGKTELNYKNTEPVLDSDGSYFMYSK | |
| | LRVEKKNWVERNSYSCSVVHEGLHNHHTTKSFSRTPGK | |
| S3F full | DIVMTQSHKFMSTSVGDRVSITCKASQDVGTNIAWYQQKP | 64 |
| length light | GRSPKVLIYSASTRHTGVPDRFTGSGSGTDFTLTISNVQSED | |
| chain | LTDYFCQQYSSFPLTFGVGTKLELKRADAAPTVSIFPPSSEQ | |
| | LTSGGASVVCFLNNFYPKDINVKWKIDGSERQNGVLNSWT | |
| | DQDSKDSTYSMSSTLTLTKDEYERHNSYTCEATHKTSTSPI | |
| | VKSFNRNEC | |
| S3F full | ATGGAGACCGACACCCTGCTGCTCTGGGTGCTGCTGCTC | 65 |
| length heavy | TGGGTGCCCGGCTCCACCGGACAGGTTCAGCTGCAGCAG | |
| chain | TCTGACGCTGAGTTGGTGAAACCTGGGGCTTCAGTGAAG | |
| nucleotide | ATATCCTGCAAGGCTTCTGGCTACACCTTCACTGACCAT | |
| sequence | GCTATTCACTGGGTGAAGCAAAAGCCTGAACAGGGCCTG | |
| | GACTGGATTGGATATATTTCTCCCGGAAATGGTGATATT | |
| | AAGTACAATGAGAAGTTCAAGGACAAGGTCACACTGAC | |
| | TGCAGACAAATCCTCCAGCACTGCCTGCATGCACCTCAA | |
| | CAGCCTGACATCTGAGGATTCTGCAGTGTATTTCTGCAA | |
| | AAGATCCCTACTAGCTCTTGACTACTGGGGCCAAGGCAC | |
| | CACTCTCACAGTCTCCTCAGCTAAAACAACAGCCCCATC | |
| | GGTCTATCCACTGGCCCCTGTGTGTGGAGATACAACTGG | |
| | CTCCTCGGTGACTCTAGGATGCCTGGTCAAGGGTTATTTC | |
| | CCTGAGCCAGTGACCTTGACCTGGAACTCTGGTTCCCTG | |
| | TCCAGTGGTGTGCACACCTTCCCAGCTGTCCTGCAGTCTG | |
| | ACCTCTACACCCTCAGCTCAAGCGTGACTGTAACCAGCT | |
| | CGACCTGGCCCAGCCAGTCCATCACCTGCAATGTGGCCC | |
| | ACCCGGCAAGCAGCACCAAGGTGGACAAGAAAATTGAG | |
| | CCCAGAGGGCCCACAATCAAGCCCTGTCCTCCATGCAAA | |
| | TGCCCAGCACCTAACCTCTTGGGTGGACCATCCGTCTTC | |
| | ATCTTCCCTCCAAAGATCAAGGATGTACTCATGATCTCC | |
| | CTGAGCCCCATAGTCACATGTGTAGTCGTTGATGTGAGC | |
| | GAGGATGACCCAGATGTCCAGATCAGCTGGTTTGTGAAC | |
| | AACGTGGAAGTGCACACTGCTCAGACACAGACGCATAG | |
| | AGAGGATTACAACAGTACTCTCCGGGTTGTCAGTGCCCT | |
| | CCCCATCCAGCACCAGGACTGGATGAGTGGCAAGGAGTT | |
| | CAAATGCAAGGTCAACAACAAAGACCTCCCAGCGCCCA | |
| | TCGAGAGAACCATCTCAAAACCCAAAGGGTCAGTAAGA | |
| | GCTCCACAGGTATATGTCTTGCCTCCACCAGAAGAGGAG | |
| | ATGACTAAGAAACAGGTCACTCTGACCTGCATGGTCACA | |
| | GACTTCATGCCTGAAGACATTTACGTGGAGTGGACCAAC | |
| | AACGGGAAAACAGAGCTAAACTACAAGAACACTGAACC | |
| | AGTCCTGGACTCTGATGGTTCTTACTTCATGTACAGCAA | |
| | GCTGAGAGTGGAGAAGAAGAACTGGGTGGAGAGAAATA | |
| | GCTACTCCTGTTCAGTGGTCCACGAGGGTCTGCACAATC | |
| | ACCACACGACTAAGAGCTTCTCCCGGACTCCGGGTAAAT | |
| | AG | |
| S3F full | ATGGAGACCGACACCCTGCTGCTCTGGGTGCTGCTGCTC | 66 |
| length light | TGGGTGCCCGGCTCCACCGGAGACATTGTGATGACCCAG | |
| chain | TCTCACAAATTCATGTCCACATCAGTAGGAGACAGGGTC | |
| nucleotide | AGCATCACCTGCAAGGCCAGTCAGGATGTGGGCACTAAT | |
| sequence | ATAGCCTGGTATCAACAGAAACCAGGCCGATCTCCTAAA | |
| | GTACTGATTTACTCGGCATCCACCCGGCACACTGGAGTC | |
| | CCTGATCGCTTCACAGGCAGTGGATCTGGGACAGATTTC | |
| | ACTCTCACCATTAGCAATGTGCAGTCTGAAGACTTGACA | |
| | GATTATTTCTGTCAGCAATATAGCAGCTTTCCTCTCACGT | |
| | TCGGTGTTGGGACCAAGCTGGAGCTGAAACGGGCAGAT | |
| | GCTGCACCAACTGTATCCATCTTCCCACCATCCAGTGAG | |
| | CAGTTAACATCTGGAGGTGCCTCAGTCGTGTGCTTCTTG | |
| | AACAACTTCTACCCCAAAGACATCAATGTCAAGTGGAAG | |
| | ATTGATGGCAGTGAACGACAAAATGGCGTCCTGAACAGT | |
| | TGGACTGATCAGGACAGCAAAGACAGCACCTACAGCAT | |
| | GAGCAGCACCCTCACGTTGACCAAGGACGAGTATGAAC | |
| | GACATAACAGCTATACCTGTGAGGCCACTCACAAGACAT | |
| | CAACTTCACCCATTGTCAAGAGCTTCAACAGGAATGAGT | |
| | GTTGA | |

Plasmids encoding S3F full heavy chain amino acid sequences and plasmids encoding S3F full light chain amino acid sequences were transfected into Chinese hamster ovary-K1 (CHO-K1) cells for the generation of stable cell lines expressing S3F IgG2a antibodies. The cells were cultured in a humidified 5% C02 incubator at 37° C. in chemically defined media (CD-CHO, Invitrogen, Carlsbad, CA) supplemented with L-glutamine.

Approximately 80 million suspension CHO cells, growing in log phase, were transfected by electroporation (MaxCyte) with 80 μg of total plasmid encoding the full length heavy and light chains of S3F. Twenty four hours later, the transfected cells were placed under selection for stable integration of the antibody genes. During the selection process the cells were spun down and resuspended in fresh selection media every 2-3 days until the pool recovered its growth rate and viability. Cells were monitored for growth, titer, and stable integration of the antibody expression constructs. The doubling rate was 20 hours.

Two small scale production scale-ups were performed using the stably transfected cells. The cells were scaled up for production in OptiCHO CD Growth Medium (Invitrogen). The product was produced at a titer of approximately 12 mg per liter. The doubling rate was 20 hours. The conditioned media supernatant harvested from the transient transfection production run was clarified by centrifuge spinning. The protein was run over a Protein A column and eluted using two different buffer formulations (Citrate Buffer and HEPES buffer). Filtration using a 0.2 μm membrane filter was performed. Size exclusion chromatography (SEC) was performed for both formulations (see the following Table).

TABLE 24
SEC data

| Buffer | Results |
|---|---|
| 20 mM Citrate (pH 5.5) + | Purity 91.4%, |
| 150 mM NaCl | aggregates 3.4% |
| 200 mM HEPES (pH 7) + | Purity 96.2%, |
| 0.2% acetate | aggregates 3.8% |

Stable cell lines were cultured for large scale production and 10 L of culture were produced. The conditioned media harvested from the stable cell pool production run was clarified by centrifugation and 0.2 μm membrane filtration. The antibody was purified using Protein A affinity chromatography, then sterilized and cleared of particulates by passing through a 0.2 μm membrane filter. After low endotoxin purification and filtration, concentration was set to 5 mg/mL and 120 mg of antibody S3F was recovered.

### Example 35. Internalization Assay Using Labeled S3F

Culturing and harvesting of MDA-STn+ cells was performed according to Example 14. Staining of MDA-STn+ cells with S3F antibody included either unlabeled S3F or S3F directly labeled with ALEXA FLUOR® 488 (Thermo Fisher Scientific, Pittsburgh, PA). Stained control vials were kept at 4° C. until analysis was performed. Controls included a GB26.6 antibody negative control for STn binding, unstained control, and unlabeled S3F antibody followed by an APC secondary antibody. Vials for each experimental time point (15, 30 and 60 min) were incubated in complete media at 37° C. 5% C02 to allow for antibody internalization. Immediately prior to analysis by flow cytometry, non-internalized antibody was stripped from the cell surface of experimental samples using 150 mM NaCl pH 2.5 (acidic buffer).

Unlabeled S3F followed by an APC secondary antibody stained ˜98% of MDA-STn+ cells. S3F antibody directly labeled with ALEXA FLUOR® 488 stained ˜50-57% of MDA-STn+ cells, suggesting that the conjugation of S3F to ALEXA FLUOR® 488 dyes may inhibit S3F antibody binding. Control samples treated with 150 mM NaCl pH 2.5 acidic buffer displayed an 80% reduction of fluorescence signal as compared to control samples not treated with 150 mM NaCl pH 2.5 acidic buffer.

After 15 minutes of incubation at 37° C. 5% C02 and stripping of non-internalized antibody by 150 mM NaCl pH 2.5 acidic buffer, 22.5% of MDA-STn+ cells were stained with S3F directly labeled with ALEXA FLUOR® 488. The percentage of cells stained with S3F directly labeled with ALEXA FLUOR® 488 increased to 31.5% after 60 minutes of incubation at 37° C. 5% C02 and stripping of non-internalized antibody by 150 mM NaCl pH 2.5 acidic buffer, indicating the S3F antibody is being internalized.

### Example 36. Fluorescence Microscopy Analysis of Antibody Internalization

To analyze the internalization of antibodies by fluorescence microscopy, the following procedure was carried out. MDA-MD-231 STn− cells and MDA-MD-231 STn+ cells were cultured in Eagles minimum essential medium containing 10% fetal calf serum, 100 μg/ml penicillin, 100 UI/ml streptomycin, 45 μg/ml gentamycin, as well as 1 mg/ml G418 for STn+ cells. Cells were sub-cultured by treatment with Accutase buffer and seeded in microscope chamber slides at 10,000 cells/chamber in the case of STn− cells and 20,000 cells/chamber in the case of STn+ cells. All cells were cultured overnight at 37° C. Cells were subsequently incubated with either 1.5, 5 or 10 μg/ml S3F conjugated with ALEXA FLUOR® 488 dye (Thermo Fisher Scientific, Pittsburgh, PA) in complete media at 4° C. for 1 hour. Cells were then washed with ice-cold PBS in order to remove antibodies that were not bound to the cell surface. Complete media was then administered to each chamber and the cells were incubated at 37° C. for either 0, 15, 30 or 60 minutes in order to allow internalization of any surface-bound antibodies.

Following each time point, cells were washed twice with PBS in order to remove unbound antibodies. Cells were then washed twice with acidic solution (150 mM NaCl/HCl in mQ water, pH=2.5) to remove surface-bound antibodies. Cells were then washed with PBS and fixed by treatment with a solution of 3% paraformaldehyde and 2% sucrose at room temperature for 15 minutes. Cells were again washed with PBS and subsequently incubated with DAPI at room temperature for 5 minutes so as to fluorescently label nuclear DNA. Following this incubation, cells were washed with PBS and treated with mounting medium and allowed to dry overnight. Cells were visualized the following day by analysis on a Nikon Eclipse Ti fluorescence microscope.

The results of this analysis revealed that antibody S3F binds the surface of STn+ cells and does not bind the surface of STn− cells. Incubation of STn+ cells with 488-S3F conjugate at 1.5, 5 and 10 μg/ml without washing of the cells with acidic solution resulted in a vibrant fluorescence pattern across each cell, while little to no green fluorescence was observed following the same treatment of STn− cells. In the case of STn+ cells, the fluorescence intensity appeared to be evenly distributed about the entire cell, an observation that is consistent with cell-surface binding. Treatment of STn+ cells with 1.5 μg/ml ALEXA FLUOR® 488-S3F conjugate without an ensuing incubation at 37° C. followed by immediate removal of surface-bound antibodies via acidic treatment resulted in the observation of little to no green fluorescence associated with the cells, and only dim green fluorescence was observed for STn+ cells treated with 5 or 10 μg/ml antibody. No green fluorescence was observed for STn− cells treated with 1.5, 5 or 10 μg/ml ALEXA FLUOR® 488-S3F in the absence of an incubation at 37° C. and following an acid wash. Taken together, these results suggest the S3F antibody is capable of binding the surface of STn+ cells and does not effectively bind the surface of STn− cells.

The analysis also revealed that antibody S3F is capable of being internalized by STn+ cells, but is not efficiently internalized by STn− cells. STn+ cells that were incubated with 1.5, 5 or 10 μg/ml ALEXA FLUOR® 488-S3F began to exhibit green fluorescence after incubation at 37° C. for 15 minutes and subsequent treatment with acidic solution to remove surface-bound antibodies. For STn+ cells incubated with each concentration of antibody, the intensity of green fluorescence increased when the 37° C. incubation time was increased from 15 to 30 minutes prior to the acid wash, indicating internalization of the antibody was indeed mediated by surface-bound S3F. The fluorescence distribution of the STn+ cells was also consistent with internalization, as green fluorescence was typically localized to discrete, individual regions within the cell amidst a background of less intense, diffuse fluorescence. This observation is consistent with endosomal entry of the antibody, followed by subsequent endosomal escape. In all cases, little to no green fluorescence was observed for STn− cells treated with varying ALEXA FLUOR® 488-S3F concentrations and incubated at 37° to allow internalization to occur prior to acid treatment. Collectively, these data suggest that S3F selectively binds the surface of STn+ cells and is subsequently internalized in a time-dependent fashion.

### Example 37. Antibody Characterization Summary

Based on characterization data, S3F antibody was categorized by anti-STn group number according to Example 13. The following Table shows the antibody groups in which S3F is thought to belong based on flow cytometry analysis and glycan array analysis.

TABLE 25
S3F antibody characterization summary

| | | Flow cytometry | | | | | |
|---|---|---|---|---|---|---|---|
| | | (EC50) | | Glycan Array | | | |
| Antibody | Isotype | MDA-STn+ | MDA-STn− | AcSTn | GcSTn | STn Specificity | Group |
| S3F | IgG2a κ | 1.06 | NB | 63% | 31% | 94% | Group 1 |

### Example 38. Immunohistochemical Staining Procedures

Immunohistochemical (IHC) staining is performed using anti-STn antibodies with and without neuraminidase treatment. The specimens may include normal and cancerous tissue samples.

The procedure for IHC staining includes the following steps: a). obtain tissue samples and prepare specimen slides; b). prepare tissue samples for IHC staining; i. perform antigen retrieval with Diva antigen retrieval solution for 30 minutes at 95° C.; or treat slides with 250 mU/mL neuraminidase in 50 mM sodium acetate, pH 5.5 for 2.5 hours at 37° C.; ii. rinse all treated slides twice with Tris-buffered saline auto-wash (TBS-AW); iii. apply Sniper protein block to all slides for 20 minutes to reduce non-specific binding and rinse twice in TBS-AW; c). perform antibody staining; i. incubate the pretreated slides with the primary antibody. For example, anti-STn antibody S3F is applied (10 or 30 ug/mL) for 60 minutes; ii. rinse twice in TBS-AW; iii. block endogenous peroxidase with peroxidase 1 for 10 minutes; iv. rinse twice with TBS-AW; v. incubate the slides with biotinylated goat anti-mouse IgG in DaVinci Green at 1:500 dilution for 30 minutes; vi. rinse twice with TBS-AW. d). develop the staining; i. add ABC Elite and incubate the slides for 30 minutes; ii. rinse twice with TBS-AW; iii. apply Sigma Fast DAB solution for 5 minutes followed by a rinse in distilled water; iv. counterstain and coverslip the slides for analysis.

The following reagents are used for IHC staining: Diva antigen retrieval solution (Biocare Medical, Concord CA); Tris-buffered saline Auto-Wash (TBS-AW)(Biocare Medical, Concord CA); Neuraminidase in 50 mM sodium acetate, pH 5.5 (EY Laboratories/Sigma, San Mateo, CA); Peroxidazed 1 (Biocare Medical, Concord CA); ABC Elite (Vector Laboratories, Burlingame, CA); Biotinylated goat anti-mouse IgG (Jackson Immunoresearch, West Grove, PA); DaVinci Green antibody diluent (Biocare Medical, Concord CA) and Sigma Fast 3,3′ Diaminobenzidine (DAB) Solution (Sigma-Aldrich).

### Example 39. Immunohistochemistry of Normal and Tumor Tissues Using Antibodies S3F and 7G9

To assess the properties of anti-STn antibodies, IHC staining of normal and tumor tissues using anti-STn mAbs S3F and 7G9 were performed. All the staining experiments were carried out according to the procedure described above. Neuraminidase pretreatment was used to prevent non-specific staining.

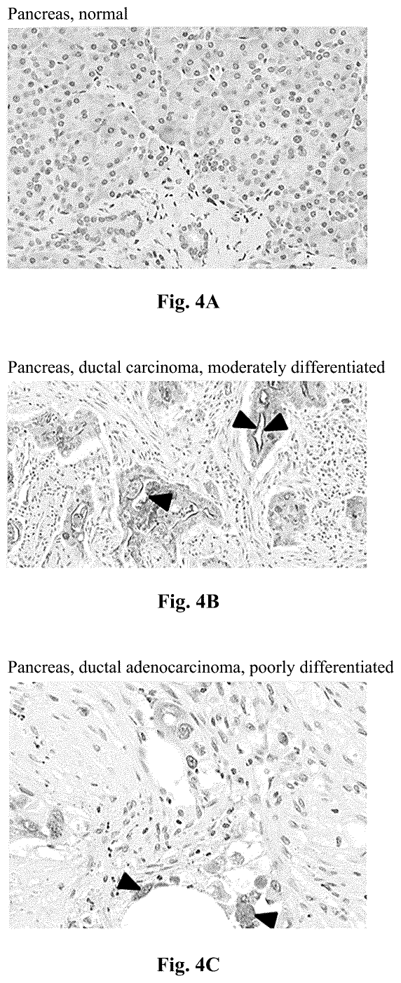

S3F IHC staining was performed using tissue microarray (TMA) slides containing normal and neuroplastic tissue specimens. Such specimens included tissue samples from different sections of colon such as ascending colon and sigmoid colon; rectum; ovary; pancreas; prostate; lung and breast. The observed staining patterns indicated that STn was present in most cancerous tissues such as colorectal cancers, poorly differentiated ovarian adenocarcinoma, serious ovarian cystadenocarcinoma, ovarian mucinous cystadenoma, ovarian serious papillary cystadenoma, pancreatic ductal carcinoma, pancreatic ductal adenocarcinoma, prostatic adenocarcinoma, lung adenocarcinoma, lung squamous cell carcinoma, breast infiltrating duct carcinoma, and breast medullary carcinoma. STn expression was rare in normal tissues. FIG. 4 depicts an example of staining, using S3F, in normal pancreas (FIG. 4A) and cancerous pancreas (FIGS. 4B and 4C). Arrows indicate positively stained cells.

### Example 40. ADCs in In Vitro

S3F antibodies were developed into antibody drug conjugates (ADCs) by attachment of monomethyl auristatin E (MMAE) or monomethyl auristatin F (MMAF) conjugates with a cleavable (CL) or non-cleavable linker (NC). ADCs were tested by treating MDA-MB-231 parental cells (control) or MDA-MB-231-STn cells. Cell viability with increasing concentrations of antibody was determined using the CELLTITER-GLO® luminescent cell viability assay (Promega, Madison, WI) and used to calculate half maximal inhibitory values (IC₅₀). An IC₅₀ of 0.74 was observed in MDA-MB-231-STn cells treated with CL-MMAE-conjugated S3F and an IC₅₀ of 0.89 was observed in MDA-MB-231-STn cells treated with NC-MMAF-conjugated S3F.

### Example 41. ADCs as Single Agents in the MDA-MB-231-STn Human Breast Cancer Xenograft Model

To evaluate antibody drug conjugates (ADCs) as single agents in an MDA-MB-231-STn Human Breast Tumor Xenograft model, 40 ICR SCID female mice age range 6-8 weeks were randomly separated into four separate groups, one vehicle control and three ADC treatments, then maintained individually with food/water ad libitum (see the following Table).

TABLE 26
ADC treatments

| Treatment | |
|---|---|
| Number | Treatment |
| 1 | Vehicle Control |
| 2 | S3F-IgG2a |
| 3 | S3F-CL-MMAE |
| 4 | S3F-NC-MMAF |

Animals were inoculated in the subcutaneous right flank with 5×10⁶ breast tumor cells (MDA-MB-231-STn) in matrigel, mean tumor size was 175-225 mm³ at study initiation. Animals were given either vehicle or antibody drug conjugates (5 mg/kg) by intraperitoneal injection once weekly. Tumor volume and percent body weight change were determined once weekly until study termination. The study was terminated when the tumor in the vehicle control animal reached a mean tumor weight >1500 mm³. Results are presented in the following Table. SE refers to “standard error.”

TABLE 27
Tumor volume changes with treatment

| | Vehicle | Vehicle | S3F- | S3F- | | | | |
|---|---|---|---|---|---|---|---|---|
| | Control | Control | IgG2a | IgG2a | S3F-MMAE | S3F-MMAE | S3F-MMAF | Volume |
| | Tumor | Tumor | Tumor | Tumor | Tumor | Tumor | Tumor | S3F-MMAF |
| | Volume | Volume | Volume | Volume | Volume | Volume | Volume | Tumor |
| Day | (mm3) | SE | (mm³) | SE | (mm³) | SE | (mm³) | SE |
| 1 | 179.0 | 24.9 | 178.6 | 23.8 | 178.6 | 21.6 | 178.6 | 21.8 |
| 4 | 209.7 | 28.8 | 194.3 | 32.0 | 153.6 | 19.2 | 214.1 | 23.4 |
| 8 | 258.0 | 36.0 | 244.9 | 38.5 | 100.0 | 9.8 | 245.5 | 26.7 |
| 10 | 281.7 | 39.6 | 301.0 | 65.2 | 68.4 | 10.8 | 246.1 | 28.3 |
| 15 | 366.9 | 57.2 | 445.1 | 130.2 | 30.3 | 4.5 | 267.3 | 49.0 |
| 18 | 481.7 | 76.9 | 609.7 | 175.1 | 21.6 | 3.5 | 383.6 | 75.8 |
| 23 | 704.3 | 139.4 | 775.4 | 206.0 | 8.9 | 3.0 | 461.8 | 85.3 |
| 25 | 730.8 | 151.3 | 972.7 | 307.5 | 4.7 | 1.8 | 484.8 | 105.3 |

Total tumor volume increased at all time points in animals treated with vehicle control, S3F-IgG2a or S3F-NC-MMAF. In sharp contrast the antibody drug conjugate S3F-MMAE treated tumors decreased starting with the first time point at day four and continuing throughout the study duration. Additionally S3F-NC-MMAF showed a slower rate of tumor growth when compared with vehicle control.

All animals showed an initial decrease in body weight with a later increase in body weight over the duration of the study. At study termination animals treated with either vehicle control or S3F-IgG2a showed an overall increase in body weight as noted by positive changes in body weight percent. In contrast animals treated with S3F-CL-MMAE or S3F-NC-MMAF showed slightly decreased body weight percentages at study termination (see the following Table).

TABLE 28
Percent Body Weight Change with Treatment

| Study | Vehicle | S3F- | S3F- | S3F- |
|---|---|---|---|---|
| Day | Control | IgG2a | MMAE | MMAF |
| 1 | 0 | 0 | 0 | 0 |
| 4 | −1.98 | −0.01 | −0.04 | −0.04 |
| 8 | −0.94 | −1.29 | −2.39 | −1.65 |
| 10 | −0.09 | −1.36 | −2.90 | −2.97 |
| 15 | −0.32 | −1.09 | −2.66 | −2.43 |
| 18 | 0.76 | −0.51 | −3.13 | −1.54 |
| 23 | 0.97 | 1.49 | −0.77 | −0.32 |
| 25 | 1.48 | 2.17 | −0.57 | 0.61 |

Clinical observations of tumor shape, size and disposition were noted throughout the study. Tumors in several of the vehicle control animals exhibited necrosing tumors with secreted fluid ranging from slight to moderate in severity. Animals treated with S3F-IgG2a had fewer severe tumors with fluid secretion as compared with vehicle control although tumors still ranged from slight to moderate in severity. S3F-CL-MMAF treated animals had slight tumors with no fluid extrusion. Alternatively, tumors in animals treated with S3F-NC-MMAE became flattened and had no sign of fluid extrusion. Clinical observation found that tumors were completely abolished in the majority (9 out of 10) of these animals.

Mice belonging to treatment group 3 (receiving S3F-CL-MMAE) were monitored through day 54 of the study (more than 30 days beyond their last antibody treatment). Results are presented in the following Table.

TABLE 29
Continued monitoring of S3F-CL-MMAE mice

| | Tumor | Tumor | % Body |
|---|---|---|---|
| | Volume | Volume | Weight |
| Day | (mm³) | SE | Change |
| 29 | 5.6 | 2.6 | −1.28 |
| 32 | 5.0 | 2.6 | 3.00 |
| 37 | 8.1 | 4.6 | 1.65 |
| 40 | 7.1 | 4.7 | 3.03 |
| 44 | 5.6 | 5.2 | 1.25 |
| 47 | 5.2 | 4.4 | 1.65 |
| 51 | 7.3 | 6.3 | 0.17 |
| 54 | 11.2 | 9.1 | 4.04 |

Surprisingly, tumor volume remained low in these mice and body weight increased overall.

### Example 42. ADCs as Single Agents in the COLO-205 Human Colon Adenocarcinoma Tumor Xenograft Model

To evaluate antibody drug conjugates (ADCs) as single agents in an COLO-205 Colon AdenocarcinomaTumor Xonograft model, 40 athymic nude female mice age range 6-8 weeks were randomly separated into four separate groups, one vehicle control and three ADC treatments, then maintained individually with food/water ad libitum (see the following Table).

TABLE 30
ADC treatments

| Treatment | |
|---|---|
| Number | Treatment |
| 1 | Vehicle Control |
| 2 | S3F-IgG2a |
| 3 | S3F-CL-MMAE |
| 4 | S3F-NC-MMAF |

Animals were inoculated in the subcutaneous right flank with 5×10⁶ colon adenocarcinoma tumor cells (COLO-205) in matrigel, mean tumor size was 175-225 mm³ at study initiation. Animals were given either vehicle or one of the antibody drug conjugates (5 mg/kg) by intraperitoneal injection once weekly. Tumor volume and percent body weight change were determined once weekly until study termination. The study was terminated when the tumor in the vehicle control animal reached a mean tumor weight >1500 mm³. Results are presented in the following Table. SE refers to “standard error.”

TABLE 31
Tumor Volume Changes with Treatment

| | Vehicle | Vehicle | S3F- | S3F- | S3F-CL- | S3F-CL- | S3F-NC- | S3F-NC- |
|---|---|---|---|---|---|---|---|---|
| | Control | Control | IgG2a | IgG2a | MMAE | MMAE | MMAF | MMAF |
| | Tumor | Tumor | Tumor | Tumor | Tumor | Tumor | Tumor | Tumor |
| Study | Volume | Volume | Volume | Volume | Volume | Volume | Volume | Volume |
| Day | (mm3) | SE | (mm³) | SE | (mm³) | SE | (mm³) | SE |
| 1 | 183.1 | 15.4 | 182.8 | 15.7 | 183.1 | 13.1 | 182.9 | 13.5 |
| 5 | 323.8 | 35.7 | 315.2 | 20.5 | 240.1 | 26.3 | 308.5 | 31.3 |
| 8 | 429.1 | 41.2 | 448.8 | 32.2 | 299.5 | 36.5 | 442.8 | 38.5 |
| 10 | 533.5 | 44.5 | 543.1 | 30.3 | 275.7 | 41.7 | 571.1 | 51.8 |
| 15 | 688.7 | 47.6 | 655.3 | 67.2 | 333.9 | 67.3 | 678.4 | 67.8 |
| 19 | 809.9 | 59.5 | 850.6 | 99.0 | 360.4 | 76.0 | 804.9 | 87.6 |
| 22 | 857.3 | 100.1 | 938.2 | 93.0 | 355.0 | 80.2 | 839.9 | 117.6 |
| 25 | 832.2 | 127.2 | 1001.4 | 116.5 | 370.1 | 85.9 | 893.0 | 151.9 |

Total tumor volume increased in all animals between days 1 and 8. After day 8 animals treated with vehicle control, S3F-IgG2a, and S3F-NC-MMAF continued to show rapid increase in volume. Animals treated with S3F-CL-MMAE showed a plateau in growth starting at day 8 and maintaining a lower rate of tumor growth when compared with all other treatment groups.

Body weight changes (shown in the following Table) were seen in all animals. Initial drops in body weight were seen in all treatment groups. Animals treated with vehicle control, S3F-IgG2a, and S3F-NC-MMAF remained low with only S3F-NC-MMAF treated animals regaining body weight back to normal range by the study termination. In contrast, animals treated with S3F-CL-MMAE showed a rapid rise in body weight following the initial losses out to day 15, showing a total weight gain over the study time of 7.26%.

TABLE 32
Percent Body Weight Change with Treatment

| Study | Vehicle | S3F- | S3F-CL- | S3F-NC- |
|---|---|---|---|---|
| Day | Control | IgG2a | MMAE | MMAF |
| 1 | 0 | 0 | 0 | 0 |
| 5 | 0.84 | 1.18 | 1.91 | 2.82 |
| 8 | −1.68 | −5.01 | 0.23 | −2.99 |
| 10 | −0.97 | −2.53 | −0.42 | −2.39 |
| 15 | −3.14 | −5.22 | 4.65 | −3.39 |
| 19 | −4.25 | −1.33 | 5.72 | 0.12 |
| 22 | −4.47 | −0.91 | 5.63 | 1.47 |

Clinical observations of tumor shape, size and disposition were noted throughout the study. Tumors in the vehicle control, S3F-IgG2a and S3F-NC-MMAF treated animals exhibited signs of illness including necrosing tumors ranging in severity from slight to severe. In addition some animals were emaciated and showed hunched posture and severe pallor. Tumors in animals treated with S3F-CL-MMAE, on the other hand, became flattened and only one animal showed signs of hunched posture. None of the animals treated with S3F-CL-MMAE showed signs of emaciation or pallor.

1.-42. (canceled)

43. An isolated antibody that binds to sialyl(α2,6)N-acetylgalactosamine (STn), wherein the antibody comprises:
a) a CDR-H1 comprising the amino acid sequence of SEQ ID NO: 15, a CDR-H2 comprising the amino acid sequence of SEQ ID NO: 19, a CDR-H3 comprising the amino acid sequence of SEQ ID NO: 22, a CDR-L1 comprising the amino acid sequence of SEQ ID NO: 31 or 32, a CDR-L2 comprising the amino acid sequence of SEQ ID NO: 33 or 35, and a CDR-L3 comprising the amino acid sequence of SEQ ID NO: 36 or 40;
b) a CDR-H1 comprising the amino acid sequence of SEQ ID NO: 16, a CDR-H2 comprising the amino acid sequence of SEQ ID NO: 20, a CDR-H3 comprising the amino acid sequence of SEQ ID NO: 23, a CDR-L1 comprising the amino acid sequence of SEQ ID NO: 29, a CDR-L2 comprising the amino acid sequence of SEQ ID NO: 33, and a CDR-L3 comprising the amino acid sequence of SEQ ID NO: 36;
c) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 24, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 37;
d) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 25, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38;
e) a CDR-H1 comprising the amino acid sequence of SEQ ID NO: 18, a CDR-H2 comprising the amino acid sequence of SEQ ID NO: 21, a CDR-H3 comprising the amino acid sequence of SEQ ID NO: 26, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38;
f) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 27, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38; or
g) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 28, a CDR-L1 comprising the amino acid sequence of SEQ ID NO: 30, a CDR-L2 comprising the amino acid sequence of SEQ ID NO: 34, and a CDR-L3 comprising the amino acid sequence of SEQ ID NO: 39.

44. The isolated antibody of claim 43, wherein the antibody comprises:
a) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 1, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 2 or 3; or
b) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 4, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 5;
c) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 6, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 7;
d) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 8, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 9;
e) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 10, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 11;
f) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 12, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 9; or
g) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 13, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 14.

45. The isolated antibody of claim 44, wherein the antibody comprises:
a) a heavy chain variable region (VH) comprising the amino acid sequence of SEQ ID NO: 1, and a light chain variable region (VL) comprising the amino acid sequence of SEQ ID NO: 2 or 3; or
b) a heavy chain variable region (VH) comprising the amino acid sequence of SEQ ID NO: 4, and a light chain variable region (VL) comprising the amino acid sequence of SEQ ID NO: 5;
c) a heavy chain variable region (VH) comprising the amino acid sequence of SEQ ID NO: 6, and a light chain variable region (VL) comprising the amino acid sequence of SEQ ID NO: 7;
d) a heavy chain variable region (VH) comprising the amino acid sequence of SEQ ID NO: 8, and a light chain variable region (VL) comprising the amino acid sequence of SEQ ID NO: 9;
e) a heavy chain variable region (VH) comprising the amino acid sequence of SEQ ID NO: 10, and a light chain variable region (VL) comprising the amino acid sequence of SEQ ID NO: 11;
f) a heavy chain variable region (VH) comprising the amino acid sequence of SEQ ID NO: 12, and a light chain variable region (VL) comprising the amino acid sequence of SEQ ID NO: 9; or
g) a heavy chain variable region (VH) comprising the amino acid sequence of SEQ ID NO: 13, and a light chain variable region (VL) comprising the amino acid sequence of SEQ ID NO: 14.

46. The isolated antibody of claim 43, wherein the antibody comprises a human IgG1, IgG2a, IgG2b, IgG2c, IgG3, or IgG4 constant region.

47. The isolated antibody of claim 43, wherein the antibody binds to cell-associated STn with a half maximal effective concentration (EC50) of from about 0.01 nM to about 10 nM.

48. A pharmaceutical composition comprising the antibody of claim 43 and at least one pharmaceutically acceptable excipient.

49. One or more isolated nucleic acids that encode the antibody of claim 43.

50. A host cell comprising the one or more isolated nucleic acids of claim 49.

51. A method of producing an antibody that binds to STn, comprising culturing the host cell of claim 50 under conditions suitable for expressing the antibody.

52. A method of treating cancer comprising administering to a subject with cancer a therapeutically effective amount of an antibody that binds to STn, wherein the antibody comprises:
a) a CDR-H1 comprising the amino acid sequence of SEQ ID NO: 15, a CDR-H2 comprising the amino acid sequence of SEQ ID NO: 19, a CDR-H3 comprising the amino acid sequence of SEQ ID NO: 22, a CDR-L1 comprising the amino acid sequence of SEQ ID NO: 31 or 32, a CDR-L2 comprising the amino acid sequence of SEQ ID NO: 33 or 35, and a CDR-L3 comprising the amino acid sequence of SEQ ID NO: 36 or 40;
b) a CDR-H1 comprising the amino acid sequence of SEQ ID NO: 16, a CDR-H2 comprising the amino acid sequence of SEQ ID NO: 20, a CDR-H3 comprising the amino acid sequence of SEQ ID NO: 23, a CDR-L1 comprising the amino acid sequence of SEQ ID NO: 29, a CDR-L2 comprising the amino acid sequence of SEQ ID NO: 33, and a CDR-L3 comprising the amino acid sequence of SEQ ID NO: 36;
c) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 24, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 37;
d) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 25, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38;
e) a CDR-H1 comprising the amino acid sequence of SEQ ID NO: 18, a CDR-H2 comprising the amino acid sequence of SEQ ID NO: 21, a CDR-H3 comprising the amino acid sequence of SEQ ID NO: 26, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38;
f) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 27, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38; or
g) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 28, a CDR-L1 comprising the amino acid sequence of SEQ ID NO: 30, a CDR-L2 comprising the amino acid sequence of SEQ ID NO: 34, and a CDR-L3 comprising the amino acid sequence of SEQ ID NO: 39.

53. The method of claim 52, wherein the antibody comprises:
a) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 1, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 2 or 3;
b) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 4, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 5;
c) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 6, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 7;
d) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 8, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 9;
e) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 10, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 11;
f) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 12, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 9; or
g) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 13, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 14.

54. The method of claim 52, wherein the antibody comprises:
a) a heavy chain variable region (VH) comprising the amino acid sequence of SEQ ID NO: 1, and a light chain variable region (VL) comprising the amino acid sequence of SEQ ID NO: 2 or 3; or
b) a heavy chain variable region (VH) comprising the amino acid sequence of SEQ ID NO: 4, and a light chain variable region (VL) comprising the amino acid sequence of SEQ ID NO: 5;
c) a heavy chain variable region (VH) comprising the amino acid sequence of SEQ ID NO: 6, and a light chain variable region (VL) comprising the amino acid sequence of SEQ ID NO: 7;
d) a heavy chain variable region (VH) comprising the amino acid sequence of SEQ ID NO: 8, and a light chain variable region (VL) comprising the amino acid sequence of SEQ ID NO: 9;
e) a heavy chain variable region (VH) comprising the amino acid sequence of SEQ ID NO: 10, and a light chain variable region (VL) comprising the amino acid sequence of SEQ ID NO: 11;
f) a heavy chain variable region (VH) comprising the amino acid sequence of SEQ ID NO: 12, and a light chain variable region (VL) comprising the amino acid sequence of SEQ ID NO: 9; or
g) a heavy chain variable region (VH) comprising the amino acid sequence of SEQ ID NO: 13, and a light chain variable region (VL) comprising the amino acid sequence of SEQ ID NO: 14.

55. A method of selecting a subject with cancer for treatment with an anti-STn antibody, comprising contacting a sample from the subject with the antibody of claim 43; and
detecting binding of the antibody to STn-expressing cancer cells.

56. An antibody-drug conjugate comprising an antibody conjugated to one or more therapeutic agent, wherein the antibody binds to STn and comprises:
a) a CDR-H1 comprising the amino acid sequence of SEQ ID NO: 15, a CDR-H2 comprising the amino acid sequence of SEQ ID NO: 19, a CDR-H3 comprising the amino acid sequence of SEQ ID NO: 22, a CDR-L1 comprising the amino acid sequence of SEQ ID NO: 31 or 32, a CDR-L2 comprising the amino acid sequence of SEQ ID NO: 33 or 35, and a CDR-L3 comprising the amino acid sequence of SEQ ID NO: 36 or 40;
b) a CDR-H1 comprising the amino acid sequence of SEQ ID NO: 16, a CDR-H2 comprising the amino acid sequence of SEQ ID NO: 20, a CDR-H3 comprising the amino acid sequence of SEQ ID NO: 23, a CDR-L1 comprising the amino acid sequence of SEQ ID NO: 29, a CDR-L2 comprising the amino acid sequence of SEQ ID NO: 33, and a CDR-L3 comprising the amino acid sequence of SEQ ID NO: 36;
c) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 24, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 37;
d) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 25, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38;
e) a CDR-H1 comprising the amino acid sequence of SEQ ID NO: 18, a CDR-H2 comprising the amino acid sequence of SEQ ID NO: 21, a CDR-H3 comprising the amino acid sequence of SEQ ID NO: 26, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38;
f) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 27, a CDR-L1 comprising the amino acid sequence of SEQ ID NO: 30, a CDR-L2 comprising the amino acid sequence of SEQ ID NO: 33, and a CDR-L3 comprising the amino acid sequence of SEQ ID NO: 38; or
g) a CDR-H1 comprising the amino acid sequence of SEQ ID NO: 17, a CDR-H2 comprising the amino acid sequence of SEQ ID NO: 21, a CDR-H3 comprising the amino acid sequence of SEQ ID NO: 28, a CDR-L1 comprising the amino acid sequence of SEQ ID NO: 30, a CDR-L2 comprising the amino acid sequence of SEQ ID NO: 34, and a CDR-L3 comprising the amino acid sequence of SEQ ID NO: 39.

57. The antibody-drug conjugate of claim 56, wherein the antibody comprises:
a) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 1, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 2 or 3;
b) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 4, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 5;
c) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 6, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 7;
d) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 8, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 9;
e) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 10, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 11;
f) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 12, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 9; or
g) a heavy chain variable region (VH) comprising an amino acid sequence that is at least 80%, 85%, 90%, 91%, 92%, 93%, 94%, 95%, 96%, 97%, 98%, or 99% identical to the amino acid sequence of SEQ ID NO: 13, and a light chain variable region (VL) comprising an amino acid sequence that is at least 80%, 85%, 90%, 91%, 92%, 93%, 94%, 95%, 96%, 97%, 98%, or 99% identical to the amino acid sequence of SEQ ID NO: 14.

58. The antibody-drug conjugate of claim 56, wherein the antibody comprises:
a) a heavy chain variable region (VH) comprising the amino acid sequence of SEQ ID NO: 1, and a light chain variable region (VL) comprising the amino acid sequence of SEQ ID NO: 2 or 3; or
b) a heavy chain variable region (VH) comprising the amino acid sequence of SEQ ID NO: 4, and a light chain variable region (VL) comprising the amino acid sequence of SEQ ID NO: 5;
c) a heavy chain variable region (VH) comprising the amino acid sequence of SEQ ID NO: 6, and a light chain variable region (VL) comprising the amino acid sequence of SEQ ID NO: 7;
d) a heavy chain variable region (VH) comprising the amino acid sequence of SEQ ID NO: 8, and a light chain variable region (VL) comprising the amino acid sequence of SEQ ID NO: 9;
e) a heavy chain variable region (VH) comprising the amino acid sequence of SEQ ID NO: 10, and a light chain variable region (VL) comprising the amino acid sequence of SEQ ID NO: 11;
f) a heavy chain variable region (VH) comprising the amino acid sequence of SEQ ID NO: 12, and a light chain variable region (VL) comprising the amino acid sequence of SEQ ID NO: 9; or
g) a heavy chain variable region (VH) comprising the amino acid sequence of SEQ ID NO: 13, and a light chain variable region (VL) comprising the amino acid sequence of SEQ ID NO: 14.

59. The antibody-drug conjugate of claim 56, wherein the antibody and the one or more therapeutic agent are conjugated via a cleavable linker or non-cleavable linker.

60. The antibody-drug conjugate of claim 56, wherein said therapeutic agent is a cytotoxic agent.

61. The antibody-drug conjugate of claim 56, wherein said cytotoxic agent is monomethyl auristatin E (MMAE) or monomethyl auristatin F (MMAF).

62. A method of treating cancer comprising administering to a subject with cancer a therapeutically effective amount of the antibody-drug conjugate of claim 56.

63. A method of killing a STn-expressing cell, comprising contacting the cell with an antibody-drug conjugate of claim 56.


SEQUENCE LISTING (71 sequences)

SEQ ID NO: 1 - protein, 121 aa, synthetic construct
  REGION 1..121: note = Description of Artificial Sequence: Synthetic polypeptide
QVTLKESGPGILQPSQTLSLTCSFSGFSLSTSNMGIGWIRQPSGKGLEWLAHIWWHDDKYYNPSLKSRLTISKDISNNQVFLKITSVDTADTATYYCAQVPFYYGTSFDVWGTGTTVTVSS

SEQ ID NO: 2 - protein, 107 aa, synthetic construct
  REGION 1..107: note = Description of Artificial Sequence: Synthetic polypeptide
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 3 - protein, 105 aa, synthetic construct
  REGION 1..105: note = Description of Artificial Sequence: Synthetic polypeptide
QIVLTQSPAIMSASPGETVTMTCSASSSITYMHWYQQKPGTSPKRWIYDTSKLASGVPARFSGSGSGTSYSLTISSMEAEDAATYYCHQRSSYTFGGGTKLEIKR

SEQ ID NO: 4 - protein, 121 aa, synthetic construct
  REGION 1..121: note = Description of Artificial Sequence: Synthetic polypeptide
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIAYYYGSERDYWGQGTTLTVSS

SEQ ID NO: 5 - protein, 105 aa, synthetic construct
  REGION 1..105: note = Description of Artificial Sequence: Synthetic polypeptide
QIVLTQSPAIMSASPGEKVTMTCSASSSISYMHWYHQKPGTSPKRWIYDTSKLASGVPARFSGSGSGTSYSLTISSMEAEDAATYYCHQRSSYTFGGGTKLEIKR

SEQ ID NO: 6 - protein, 112 aa, synthetic construct
  REGION 1..112: note = Description of Artificial Sequence: Synthetic polypeptide
QVQLKESGPGLVAPSQSLSITCTVSGFSLTSYGVSWVRQPPGKGLEWLGVIWGDGSTNYHSSLISRLSISKDNSKSQVFLKLNSLQTDDTATYYCARAFVYWGQGTLVTVSA

SEQ ID NO: 7 - protein, 108 aa, synthetic construct
  REGION 1..108: note = Description of Artificial Sequence: Synthetic polypeptide
QIVLTQSPAIMSASPGEKVTMTCSASSSVSYIHWYQQKSGTSPKRWIYDTSKLASGVPARFSGSGSGTSYSLTISSMEAEDAATYYCQQWSSNPPMLTFGAGTKLELK

SEQ ID NO: 8 - protein, 112 aa, synthetic construct
  REGION 1..112: note = Description of Artificial Sequence: Synthetic polypeptide
QVQLKESGPGLVAPSQSLSITCTVSGFSLTSYGVSWVRQPPGKGLEWLGVIWGDGSTNYHSALISRLIISKDNSKSQVFLKLNSLQTDDTATYYCTKGFTYWGQGTLVTVSA

SEQ ID NO: 9 - protein, 106 aa, synthetic construct
  REGION 1..106: note = Description of Artificial Sequence: Synthetic polypeptide
QIVLTQSPAIMSASPGEKVTMTCSASSSVSYMHWYQQKSGTSPKRWIFDTSKLASGVPARFSGSGSGTSYSLTISSMEAEDAATYYCQQWSSNLLTFGAGTKLELK

SEQ ID NO: 10 - protein, 112 aa, synthetic construct
  REGION 1..112: note = Description of Artificial Sequence: Synthetic polypeptide
QVQLQESGPGLVAPSQSLSITCTVSGFSLISYGVNWVRQPPGKGLEWLGVIWGDGSTNYQSALISRLIISKDNSKSQVFLKLNSLQTDDTATYYCTKGFAYWGQGTLVTVSA

SEQ ID NO: 11 - protein, 106 aa, synthetic construct
  REGION 1..106: note = Description of Artificial Sequence: Synthetic polypeptide
QIVLTQSPAIMSASPGEKVTMTCSASSSVSYMHWFQQKSGTSPKRWIYDTSKLASGVPARFSGSGSGTSYSLTISSMEAEDAATYYCQQWSSNLLTFGAGTKLELK

SEQ ID NO: 12 - protein, 112 aa, synthetic construct
  REGION 1..112: note = Description of Artificial Sequence: Synthetic polypeptide
QVQLKESGPGLVAPSQNLSITCTVSGFSLTSYGVNWVRQPPGKGLEWLGVIWGDGSTNYHSALISRLIISKENSKSQVFLKLNSLQTNDTATYYCTKGFVYWGQGTLVTVSA

SEQ ID NO: 13 - protein, 114 aa, synthetic construct
  REGION 1..114: note = Description of Artificial Sequence: Synthetic polypeptide
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAKGGYFDYWGQGTTLTVSS

SEQ ID NO: 14 - protein, 106 aa, synthetic construct
  REGION 1..106: note = Description of Artificial Sequence: Synthetic polypeptide
QIVLTQSPAVMSASPGEKVAITCSASSSVSYMHWFQQKPGTSPKLWIYSTSNLASGVPARFSGSGSGTSYSLTISRMEAEDAATYYCQQRSSYPWTFGGGTKLEIK

SEQ ID NO: 15 - protein, 10 aa, synthetic construct
  REGION 1..10: note = Description of Artificial Sequence: Synthetic peptide
GFSLSTSNMG

SEQ ID NO: 16 - protein, 10 aa, synthetic construct
  REGION 1..10: note = Description of Artificial Sequence: Synthetic peptide
GFSLSTFGMG

SEQ ID NO: 17 - protein, 8 aa, synthetic construct
  REGION 1..8: note = Description of Artificial Sequence: Synthetic peptide
GFSLTSYG

SEQ ID NO: 18 - protein, 8 aa, synthetic construct
  REGION 1..8: note = Description of Artificial Sequence: Synthetic peptide
GFSLISYG

SEQ ID NO: 19 - protein, 7 aa, synthetic construct
  REGION 1..7: note = Description of Artificial Sequence: Synthetic peptide
IWWHDDK

SEQ ID NO: 20 - protein, 7 aa, synthetic construct
  REGION 1..7: note = Description of Artificial Sequence: Synthetic peptide
IWWDDDK

SEQ ID NO: 21 - protein, 7 aa, synthetic construct
  REGION 1..7: note = Description of Artificial Sequence: Synthetic peptide
IWGDGST

SEQ ID NO: 22 - protein, 13 aa, synthetic construct
  REGION 1..13: note = Description of Artificial Sequence: Synthetic peptide
AQVPFYYGTSFDV

SEQ ID NO: 23 - protein, 13 aa, synthetic construct
  REGION 1..13: note = Description of Artificial Sequence: Synthetic peptide
ARIAYYYGSERDY

SEQ ID NO: 24 - protein, 6 aa, synthetic construct
  REGION 1..6: note = Description of Artificial Sequence: Synthetic peptide
ARAFVY

SEQ ID NO: 25 - protein, 6 aa, synthetic construct
  REGION 1..6: note = Description of Artificial Sequence: Synthetic peptide
TKGFTY

SEQ ID NO: 26 - protein, 6 aa, synthetic construct
  REGION 1..6: note = Description of Artificial Sequence: Synthetic peptide
TKGFAY

SEQ ID NO: 27 - protein, 6 aa, synthetic construct
  REGION 1..6: note = Description of Artificial Sequence: Synthetic peptide
TKGFVY

SEQ ID NO: 28 - protein, 8 aa, synthetic construct
  REGION 1..8: note = Description of Artificial Sequence: Synthetic peptide
AKGGYFDY

SEQ ID NO: 29 - protein, 5 aa, synthetic construct
  REGION 1..5: note = Description of Artificial Sequence: Synthetic peptide
SSISY

SEQ ID NO: 30 - protein, 5 aa, synthetic construct
  REGION 1..5: note = Description of Artificial Sequence: Synthetic peptide
SSVSY

SEQ ID NO: 31 - protein, 6 aa, synthetic construct
  REGION 1..6: note = Description of Artificial Sequence: Synthetic peptide
QSISDY

SEQ ID NO: 32 - protein, 5 aa, synthetic construct
  REGION 1..5: note = Description of Artificial Sequence: Synthetic peptide
SSITY

SEQ ID NO: 33-35 - skipped (3 entries)

SEQ ID NO: 36 - protein, 7 aa, synthetic construct
  REGION 1..7: note = Description of Artificial Sequence: Synthetic peptide
HQRSSYT

SEQ ID NO: 37 - protein, 11 aa, synthetic construct
  REGION 1..11: note = Description of Artificial Sequence: Synthetic peptide
QQWSSNPPMLT

SEQ ID NO: 38 - protein, 9 aa, synthetic construct
  REGION 1..9: note = Description of Artificial Sequence: Synthetic peptide
QQWSSNLLT

SEQ ID NO: 39 - protein, 9 aa, synthetic construct
  REGION 1..9: note = Description of Artificial Sequence: Synthetic peptide
QQRSSYPWT

SEQ ID NO: 40 - protein, 9 aa, synthetic construct
  REGION 1..9: note = Description of Artificial Sequence: Synthetic peptide
QNGHSFPLT

SEQ ID NO: 41 - DNA, 363 bp, synthetic construct
  misc_feature 1..363: note = Description of Artificial Sequence: Synthetic polynucleotide
  source 1..363: mol_type = other DNA
caggttactctgaaagagtctggccctgggatattgcagccctcccagaccctcagtctgacttgttctttctctgggttttcactgagcacttctaatatgggtataggctggattcgtcagccttcagggaagggtctagagtggctggcacacatttggtggcatgatgataagtactataacccatccctgaagagccggctcacaatctccaaggatatctccaacaaccaggtattcctcaagatcaccagtgtggacactgcagatactgccacgtactactgtgctcaagtcccgttttactacggaacctcgttcgatgtctggggcacagggaccacggtcaccgtctcctca

SEQ ID NO: 42 - DNA, 322 bp, synthetic construct
  misc_feature 1..322: note = Description of Artificial Sequence: Synthetic polynucleotide
  source 1..322: mol_type = other DNA
gacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttcctctcacgttcggtgctgggaccaagctggagctgaaac

SEQ ID NO: 43 - DNA, 314 bp, synthetic construct
  misc_feature 1..314: note = Description of Artificial Sequence: Synthetic polynucleotide
  source 1..314: mol_type = other DNA
caaattgttctcacccagtctccagcaatcatgtctgcatctccaggggagacggtcaccatgacctgcagtgccagctcaagtataacttacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacacgttcggaggggggaccaagctggaaataaaacg

SEQ ID NO: 44 - DNA, 363 bp, synthetic construct
  misc_feature 1..363: note = Description of Artificial Sequence: Synthetic polynucleotide
  source 1..363: mol_type = other DNA
caggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataagtactataacccagccctgaagagtcggctcacaatctccaaggatacctccaaaaaccaggtattcctcaagatcgccaatgtggacactgcagatactgccacatactactgtgctcgaatagcctattactacggtagcgagagggactactggggccaaggcaccactctcacagtctcctca

SEQ ID NO: 45 - DNA, 314 bp, synthetic construct
  misc_feature 1..314: note = Description of Artificial Sequence: Synthetic polynucleotide
  source 1..314: mol_type = other DNA
caaattgttctcacccagtctccagcaatcatgtctgcatctccaggggagaaggtcaccatgacctgcagtgccagctcaagtataagttacatgcactggtaccaccagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacacgttcggaggggggaccaagctggaaataaaacg

SEQ ID NO: 46 - DNA, 1047 bp, synthetic construct
  misc_feature 1..1047: note = Description of Artificial Sequence: Synthetic polynucleotide
  source 1..1047: mol_type = other DNA
atggacaggcttacttcctcattcttgctactgattgtccctgcatatgtcctgtcccaggttactctgaaagagtctggccctgggatattgcagccctcccagaccctcagtctgacttgttctttctctgggttttcactgagcacttctaatatgggtataggctggattcgtcagccttcagggaagggtctagagtggctggcacacatttggtggcatgatgataagtactataacccatccctgaagagccggctcacaatctccaaggatatctccaacaaccaggtattcctcaagatcaccagtgtggacactgcagatactgccacgtactactgtgctcaagtcccgttttactacggaacctcgttcgatgtctggggcacagggaccacggtcaccgtctcctcagccaaaacgacacccccatctgtctatccgctcgcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagacaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagccaggatgatcccgaggtcagttcagctgtttgtagatgatgtggaagtgcacacagctcaaaacaaccccccgagaggacatttcacaacatttccgctcatcagtgaatttcccatctgcacaagactgcttaatggcaagagttaaatgcaggtcaaagggcagtttcctgccccatcaaaaacttttcaaaa

SEQ ID NO: 47 - DNA, 702 bp, synthetic construct
  misc_feature 1..702: note = Description of Artificial Sequence: Synthetic polynucleotide
  source 1..702: mol_type = other DNA
atggattttcaagtgcagattttcagcttcctgctaatcagtgcctcagtcatactgtccagaggacaaattgttctcacccagtctccagcaatcatgtctgcatctccaggggagacggtcaccatgacctgcagtgccagctcaagtataacttacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 48 - DNA, 1252 bp, synthetic construct
  misc_feature 1..1252: note = Description of Artificial Sequence: Synthetic polynucleotide
  unsure 919: note = a, c, t, g, unknown or other
  unsure 984: note = a, c, t, g, unknown or other
  unsure 999: note = a, c, t, g, unknown or other
  unsure 1011: note = a, c, t, g, unknown or other
  unsure 1057: note = a, c, t, g, unknown or other
  unsure 1100: note = a, c, t, g, unknown or other
  unsure 1201: note = a, c, t, g, unknown or other
  source 1..1252: mol_type = other DNA
atggacaggcttacttcctcattcctgttactgattgtccctgcatatgtcctgtcccaggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataagtactataacccagccctgaagagtcggctcacaatctccaaggatacctccaaaaaccaggtattcctcaagatcgccaatgtggacactgcagatactgccacatactactgtgctcgaatagcctattactacggtagcgagagggactactggggccaaggcaccactctcacagtctcctcagccaaaacgacacccccatctgtctatccgctcgcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaagtgcacacagctcagacgncaccccggggagagcagtttcacagcactttccgctcagtcagtgaacttcccatcatgcaccangactgggctcatggncaggagttcaantgcaggtcacagtgcagctttcctgccccatcgagaaacatctccnaaacaaggcgacgaaagctcacagggtacacattccactcccnagagcaatgccagataagtcatctgactgctgatacaactcttctgaaaatactgtgaatgcatggatgccaccacgaaaatcaaacctcgcccttggacnatggcttattttaccagctagtcaaaacctggggggaatttcccgtctgtt

SEQ ID NO: 49 - DNA, 705 bp, synthetic construct
  misc_feature 1..705: note = Description of Artificial Sequence: Synthetic polynucleotide
  source 1..705: mol_type = other DNA
atggttttcacacctcagatacttggacttatgcttttttggatttcagcctccagatgtgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttcctctcacgttcggtgctgggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 50 - DNA, 336 bp, synthetic construct
  misc_feature 1..336: note = Description of Artificial Sequence: Synthetic polynucleotide
  source 1..336: mol_type = other DNA
caggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacggaagcacaaattatcattcatctctcatatccagactgagcatcagcaaggataactccaagagccaagttttcttaaaactgaacagtctgcaaactgatgacacagccacgtactactgtgccagagcctttgtttactggggccaagggactctggtcactgtctctgca

SEQ ID NO: 51 - DNA, 325 bp, synthetic construct
  misc_feature 1..325: note = Description of Artificial Sequence: Synthetic polynucleotide
  source 1..325: mol_type = other DNA
caaattgttctcacccagtctccagcaatcatgtctgcatctccaggggagaaggtcaccatgacctgcagtgccagctcaagtgtaagttacatacactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccatgctcacgttcggtgctgggaccaagctggagctgaaac

SEQ ID NO: 52 - DNA, 336 bp, synthetic construct
  misc_feature 1..336: note = Description of Artificial Sequence: Synthetic polynucleotide
  source 1..336: mol_type = other DNA
caggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgatcatcagcaaggataactccaagagccaagttttcttaaaactgaacagtctgcaaactgatgacacagccacctactactgtaccaaaggctttacttactggggccaggggactctggtcactgtctctgca

SEQ ID NO: 53 - DNA, 319 bp, synthetic construct
  misc_feature 1..319: note = Description of Artificial Sequence: Synthetic polynucleotide
  source 1..319: mol_type = other DNA
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttttgacacatccaaactggcttctggagtccctgctcgcttcagtggcagtgggtctgggacctcttactctctcacaatcagcagcatggaggctgaagatgctgccacttattactgccagcagtggagtagtaacctgctcacgttcggtgctgggaccaagctggagctgaaac

SEQ ID NO: 54 - DNA, 336 bp, synthetic construct
  misc_feature 1..336: note = Description of Artificial Sequence: Synthetic polynucleotide
  source 1..336: mol_type = other DNA
caggtgcagctgcaggagtcaggacctggcctggtggcgccctcacagagcctgtccatcacatgcactgtctcagggttctcattaatcagctatggtgtaaactgggttcgccagcctccaggaaagggtctggagtggctgggagtgatatggggtgacgggagcacaaattatcagtcagctctcatatccagactgatcatcagcaaggataactccaagagccaagttttcttaaaactgaacagtctgcaaactgatgacacagccacgtactactgtaccaaaggctttgcttactggggccaagggactctggtcactgtctctgca

SEQ ID NO: 55 - DNA, 319 bp, synthetic construct
  misc_feature 1..319: note = Description of Artificial Sequence: Synthetic polynucleotide
  source 1..319: mol_type = other DNA
caaattgttctcacccagtctccagcaatcatgtctgcatctccaggggagaaggtcaccatgacctgcagtgccagctcaagtgtaagttacatgcactggttccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacctgctcacgttcggtgctgggaccaagctggagctgaaac

SEQ ID NO: 56 - DNA, 336 bp, synthetic construct
  misc_feature 1..336: note = Description of Artificial Sequence: Synthetic polynucleotide
  source 1..336: mol_type = other DNA
caggtgcagctgaaggagtcaggacctggcctggtggcgccctcacagaacctgtccatcacatgcactgtctcagggttctcattaaccagttatggtgtaaactgggttcgccagcctccaggaaagggtctggagtggctgggagtaatatggggtgacgggagcacaaattatcattcagctctcatttccagactgatcatcagcaaggaaaactccaagagccaagttttcttaaaactgaacagtctgcaaactaatgacacagccacgtattactgtaccaaaggctttgtttactggggccaagggactctggtcactgtctctgca

SEQ ID NO: 57 - DNA, 342 bp, synthetic construct
  misc_feature 1..342: note = Description of Artificial Sequence: Synthetic polynucleotide
  source 1..342: mol_type = other DNA
caggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtgccaaagggggctactttgactactggggccaaggcaccactctcacagtctcctca

SEQ ID NO: 58 - DNA, 319 bp, synthetic construct
  misc_feature 1..319: note = Description of Artificial Sequence: Synthetic polynucleotide
  source 1..319: mol_type = other DNA
caaattgttctcacccagtctccagcagtcatgtctgcatctccaggggagaaggtcgccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccgtggacgttcggtggaggcaccaagctggaaatcaaac

SEQ ID NO: 59 - protein, 116 aa, synthetic construct
  REGION 1..116: note = Description of Artificial Sequence: Synthetic polypeptide
QVQLQQSDAELVKPGASVKISCKASGYTFTDHAIHWVKQKPEQGLDWIGYISPGNGDIKYNEKFKDKVTLTADKSSSTACMHLNSLTSEDSAVYFCKRSLLALDYWGQGTTLTVSS

SEQ ID NO: 60 - protein, 107 aa, synthetic construct
  REGION 1..107: note = Description of Artificial Sequence: Synthetic polypeptide
DIVMTQSHKFMSTSVGDRVSITCKASQDVGTNIAWYQQKPGRSPKVLIYSASTRHTGVPDRFTGSGSGTDFTLTISNVQSEDLTDYFCQQYSSFPLTFGVGTKLELK

SEQ ID NO: 61 - protein, 330 aa, synthetic construct
  REGION 1..330: note = Description of Artificial Sequence: Synthetic polypeptide
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 62 - protein, 107 aa, synthetic construct
  REGION 1..107: note = Description of Artificial Sequence: Synthetic polypeptide
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 63 - protein, 446 aa, synthetic construct
  REGION 1..446: note = Description of Artificial Sequence: Synthetic polypeptide
QVQLQQSDAELVKPGASVKISCKASGYTFTDHAIHWVKQKPEQGLDWIGYISPGNGDIKYNEKFKDKVTLTADKSSSTACMHLNSLTSEDSAVYFCKRSLLALDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 64 - protein, 214 aa, synthetic construct
  REGION 1..214: note = Description of Artificial Sequence: Synthetic polypeptide
DIVMTQSHKFMSTSVGDRVSITCKASQDVGTNIAWYQQKPGRSPKVLIYSASTRHTGVPDRFTGSGSGTDFTLTISNVQSEDLTDYFCQQYSSFPLTFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - DNA, 1401 bp, synthetic construct
  misc_feature 1..1401: note = Description of Artificial Sequence: Synthetic polynucleotide
  source 1..1401: mol_type = other DNA
atggagaccgacaccctgctgctctgggtgctgctgctctgggtgcccggctccaccggacaggttcagctgcagcagtctgacgctgagttggtgaaacctggggcttcagtgaagatatcctgcaaggcttctggctacaccttcactgaccatgctattcactgggtgaagcaaaagcctgaacagggcctggactggattggatatatttctcccggaaatggtgatattaagtacaatgagaagttcaaggacaaggtcacactgactgcagacaaatcctccagcactgcctgcatgcacctcaacagcctgacatctgaggattctgcagtgtatttctgcaaaagatccctactagctcttgactactggggccaaggcaccactctcacagtctcctcagctaaaacaacagccccatcggtctatccactggcccctgtgtgtggagatacaactggctcctcggtgactctaggatgcctggtcaagggttatttccctgagccagtgaccttgacctggaactctggttccctgtccagtggtgtgcacaccttcccagctgtcctgcagtctgacctctacaccctcagctcaagcgtgactgtaaccagctcgacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtagtcgttgatgtgagcgaggatgacccagatgtccagatcagctggtttgtgaacaacgtggaagtgcacactgctcagacacagacgcatagagaggattacaacagtactctccgggttgtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaggagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggagaagaagaactgggtggagagaaatagctactcctgttcagtggtccacgagggtctgcacaatcaccacacgactaagagcttctcccggactccgggtaaatag

SEQ ID NO: 66 - DNA, 705 bp, synthetic construct
  misc_feature 1..705: note = Description of Artificial Sequence: Synthetic polynucleotide
  source 1..705: mol_type = other DNA
atggagaccgacaccctgctgctctgggtgctgctgctctgggtgcccggctccaccggagacattgtgatgacccagtctcacaaattcatgtccacatcagtaggagacagggtcagcatcacctgcaaggccagtcaggatgtgggcactaatatagcctggtatcaacagaaaccaggccgatctcctaaagtactgatttactcggcatccacccggcacactggagtccctgatcgcttcacaggcagtggatctgggacagatttcactctcaccattagcaatgtgcagtctgaagacttgacagattatttctgtcagcaatatagcagctttcctctcacgttcggtgttgggaccaagctggagctgaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 67 - protein, 12 aa, synthetic construct
  REGION 1..12: note = Description of Artificial Sequence: Synthetic peptide
CQFDLSTRRLKC

SEQ ID NO: 68 - protein, 12 aa, synthetic construct
  REGION 1..12: note = Description of Artificial Sequence: Synthetic peptide
CQYNLSSRALKC

SEQ ID NO: 69 - protein, 10 aa, synthetic construct
  REGION 1..10: note = Description of Artificial Sequence: Synthetic peptide
EQKLISEEDL

SEQ ID NO: 70 - protein, 8 aa, synthetic construct
  REGION 1..8: note = Description of Artificial Sequence: Synthetic peptide
DYKDDDDK

SEQ ID NO: 71 - protein, 4 aa, synthetic construct
  REGION 1..4: note = Description of Artificial Sequence: Synthetic peptide
KDEL